(19) United States
(12) Patent Application Publication
(10) Pub. No.: US 2023/0146694 A1
(43) Pub. Date: May 11, 2023

(54) PEPTIDE IMMUNOGENS TARGETING PITUITARY ADENYLATE CYCLASE-ACTIVATING PEPTIDE (PACAP) AND FORMULATIONS THEREOF FOR PREVENTION AND TREATMENT OF MIGRAINE

(71) Applicant: UNITED BIOMEDICAL, INC., Hauppauge, NY (US)

(72) Inventors: Chang Yi Wang, Cold Spring Harbor, NY (US); Feng Lin, Hauppauge, NY (US); Shuang Ding, Hauppauge, NY (US)

(21) Appl. No.: 17/794,720

(22) PCT Filed: Jan. 22, 2021
(86) PCT No.: PCT/US21/14640

§ 371 Date: Jul. 22, 2022

Related U.S. Application Data
(60) Provisional application No. 62/964,953, filed on Jan. 23, 2020.

(51) Int. Cl. C07K 16/26; A61P 25/06

(52) U.S. Cl. CPC C07K 16/26; A61P 25/06

(57) ABSTRACT

The present disclosure is directed to peptide immunogen constructs targeting portions of Pituitary adenylate cyclase-activating polypeptide (PACAP), compositions containing the constructs, antibodies elicited by the constructs, and methods for making and using the constructs and compositions thereof. The disclosed peptide immunogen constructs have more than about 20 amino acids and contain (a) a B cell epitope having about more than about 9 contiguous amino acid residues from the PACAP receptor binding or activation regions of the full-length PACAP protein; (b) a heterologous Th epitope; and (c) an optional heterologous spacer. The disclosed PACAP peptide immunogen constructs stimulate the generation of highly specific antibodies directed PACAP for the prevention and/or treatment of migraine.

The present application is a PCT International Application that claims the benefit of U.S. Provisional Application Ser. No. 62/964,953, filed Jan. 23, 2020, which is incorporated herein by reference in its entirety.

### FIELD OF THE INVENTION

This disclosure relates to peptide immunogen constructs targeting pituitary adenylate cyclase-activating peptide (PACAP) and formulations thereof for prevention and treatment of pain including headache and migraine.

### BACKGROUND OF THE INVENTION

Pituitary adenylate cyclase-activating polypeptide, also known as PACAP, is a protein that, in humans, is encoded by the ADCYAP1 gene. PACAP38 and PACAP27 are produced by alternative processing from the PACAP precursor protein termed prepro-PACAP. Prepro-PACAP (GenBank Accession No. CAA42962.1) has 176 amino acids and is initially metabolized by signal proteases to generate a signal peptide (aa 1-25) and a pro-PACAP (aa 26-176). Pro-PACAP is metabolized by pro-hormone convertases and carboxy peptidases to produce a small fragment (aa 26-79), a large PACAP-Related Peptide (PRP) (aa 82-129), the physiological function of which remains unclear, and C-terminal peptides (aa 132-170 and 132-159). The C-terminal peptides are metabolized by peptidylglycine alpha-amidating monooxygenase (PAM) enzymes to form PACAP38 and PACAP27, respectively. Both PACAP38 and PACAP27 have amidated C-terminals (FIG. 1).

PACAP is similar to vasoactive intestinal peptide (VIP) and secretin. Sequence alignments of PACAP, VIP, and secretin from different species is shown in FIG. 2. PACAP binds to the VIP receptor and to the PACAP receptor. Mediated by adenylate cyclase-activating polypeptide 1 receptors, PACAP stimulates adenylate cyclase and subsequently increases the cAMP level in target cells. PACAP is a hypophysiotropic hormone (i.e., a substance that induces activity in the hypophysis) and also functions as a neurotransmitter and neuromodulator. In addition, it plays a role in paracrine and autocrine regulation of certain types of cells.

Recently a version of PACAP has been associated with post-traumatic stress disorder (PTSD) in women (but not men). This disorder involves a maladaptive psychological response to traumatic, i.e., existence-threatening, events. Ressler, K. J., et al. (2011) identified an association of a SNP in the gene coding for PACAP, implicating this peptide and its receptor (PAC1) in PTSD.

Research has shown that administration of intravenous PACAP-38 triggers delayed “migraine-like headaches” in most subjects who experience migraine headaches. Treatments with monoclonal antibodies are being developed targeting PACAP or its receptors for the treatment of primary headache disorders. These include: AMG-301 developed by Amgen Inc., which targets the PAC1 receptor and has completed phase II trials; and ALD1910, developed by Alder BioPharmaceuticals, which targets the peptide and began a phase I study in October 2019.

Although such monoclonal anti-PACAP or anti-PAC1 antibodies may prove efficacious in immunotherapy of migraine, they are expensive and must be administered monthly to maintain sufficient suppression of serum and body fluid PACAP levels and the clinical benefits derived therefrom. Cost effective immunotherapeutic treatment targeting PACAP molecule through vaccination approach that is safe and well tolerated remains an exciting new intervention and development for migraine therapies.

There are a number of disadvantages and deficiencies associated with the classical vaccines employing peptide/hapten-carrier protein immunogen preparation method. For example, the preparation methods can involve complicated chemical coupling procedures, they use expensive pharmaceutical grade KLH or toxoid protein as the T helper cell carrier, most of the antibodies elicited by the protein immunogens are directed against the carrier protein and not the target B cell epitope(s), etc.

In view of the economic and practical disadvantages and limitations with monoclonal therapy and classical vaccines employing peptide/hapten-carrier protein preparations, there is clearly an unmet need to develop an efficacious immunotherapeutic composition capable of eliciting highly specific immune responses against the functional site(s) on PACAP, that can be easily administered to patients, that can be manufactured under stringent good manufacturing practices (GMP), and is cost effective for worldwide application to treat patients suffering from migraine.

Three review articles that cite to additional supporting documents can be found for statements made in the above background section are herein incorporated by reference in their entireties. The first article contains an updated review on PACAP (website: en.wikipedia.org/wiki/Pituitary_adenylate_cyclase-activating_peptide); the second article demonstrates the extensive efforts to establish the role of the PACAP system in human biology and its therapeutic applications (Denes, V., et al., 2019); and the third article reviews the origin and function of the PACAP/glucagon superfamily (Sherwood, N. M, et al., 2000).

### REFERENCES

1. CHANG, J. C. C., et al., “Adjuvant activity of incomplete Freund's adjuvant.” Advanced Drug Delivery Reviews, 32(3):173-186 (1998)
2. DENES, V., et al., “Pituitary Adenylate Cyclase-Activating Polypeptide: 30 Years in Research Spotlight and 600 Million Years in Service” J. Clin. Med. 8(9): 1488 (2019).
3. FIELDS, G. B., et al., Chapter 3 in Synthetic Peptides: A User's Guide, ed. Grant, W.H. Freeman & Co., New York, N.Y., p. 77 (1992)
4. HIRABAYASHI, T., et al., “Discovery of PACAP and its receptors in the brain”, J. Headache Pain, 19(1):28 (2018)
5. RESSLER, K. J., et al., “Post-traumatic stress disorder is associated with PACAP and the PAC1 receptor.” Nature, 470(7335):492-497 (2011)
6. SHERWOOD, N. M, et al., “The origin and function of the pituitary adenylate cyclase-activating polypeptide (PACAP)/glucagon superfamily.” Endocrine Reviews, 21(6):619-670 (2000).
7. TRAGGIAI, E., et al., “An efficient method to make human monoclonal antibodies from memory B cells: potent neutralization of SARS coronavirus”, Nature Medicine, 10:871-875 (2004).
8. WIKIPEDIA, The free encyclopedia, “Pituitary adenylate cyclase-activating peptide” available at website: en.wikipedia.org/wiki/Pituitary_adenylate_cyclase-activating_peptide (accessed Jan. 10, 2020).
9. WO 1990/014837, by VAN NEST, G., et al., “Adjuvant formulation comprising a submicron oil droplet emulsion.” (1990 Dec. 13)

### SUMMARY OF THE INVENTION

The present disclosure is directed to portions of the Pituitary Adenylate Cyclase-Activating Peptide (PACAP) that can be used as B cell epitopes. The present disclosure is also directed to peptide immunogen constructs containing B cell epitopes from PACAP, compositions containing the peptide immunogen constructs, methods of making and using the peptide immunogen constructs, and antibodies produced by the peptide immunogen constructs.

One aspect of the present disclosure is directed B cell epitopes from different portions of PACAP from various organisms. The unstructured PACAP binds non-specifically to the cell membrane, thereby favoring the formation of a stable helical conformation at the central and C-terminal segments of the peptide. The-helix segment of PACAP interacts specifically with the N-terminal domain of the PAC1 receptor, positioning the N-terminal disordered domain of PACAP in the vicinity of the juxtamembrane domain of the receptor. The disclosed functional B cell epitope peptides have between about 9 to about 22 amino acids from the human/rat/mouse PACAP38 peptide (i.e., SEQ ID NO: 1). In certain embodiments, the functional B cell epitope peptides are located at either the N-terminal to central or C-terminal region of the PACAP molecule (e.g., SEQ ID NOs: 2-20, as shown in Table 1) where the N-terminal segment could adopt a specific conformation to activate the PAC1 receptor.

The disclosed B cell epitope peptides derived from PACAP can be linked through an optional heterologous spacer to a heterologous T helper cell (Th) epitope peptide to form a peptide immunogen construct. In certain embodiments, the heterologous spacer is any molecule or chemical structure capable of linking two amino acids and/or peptides together, which can include a chemical compound, a naturally occurring amino acid, a non-naturally occurring amino acid, or any combination thereof. The heterologous Th epitope can be any Th epitope that is capable of enhancing the immune response to the B cell epitope. In certain embodiments, the Th epitope is derived from pathogen proteins having the amino acid sequences of SEQ ID NOs: 70-109 and 160-171, as shown in Table 2.

The disclosed peptide immunogen constructs contain the PACAP B cell epitope peptide covalently linked, at either the N- or C-terminus through an optional heterologous spacer to the heterologous Th epitope. The disclosed peptide immunogen constructs, containing the B cell epitope and Th epitope, have 20 or more total amino acids. In certain embodiments, the peptide immunogen constructs have the amino acid sequences of SEQ ID NOs: 110-159, as shown in Table 3.

The disclosed PACAP peptide immunogen constructs, containing both designed B cell- and Th-epitope peptides, act together to stimulate the generation of highly specific antibodies directed against PACAP functional sites, including the PACAP receptor binding region located at the central or C-terminus of PACAP or the receptor activation region located at the N-terminus. These antibodies offer therapeutic immune responses to patients predisposed to, or suffering from, pain, including headache and migraine.

Another aspect of the present disclosure is directed to peptide compositions, including pharmaceutical compositions, containing a PACAP peptide immunogen construct. The compositions can contain one or more PACAP peptide immunogen construct, pharmaceutically acceptable delivery carriers, adjuvants, and/or be formulated into a stabilized immunostimulatory complex using a CpG oligomer. In certain embodiments, a mixture of PACAP peptide immunogen constructs have heterologous Th epitopes derived from different pathogens that can be used to allow coverage of as broad a genetic background in patients leading to a higher percentage in responder rate upon immunization for the prevention and/or treatment of patients with PACAP mediated disorders, including pain, headache, and migraine.

The present disclosure is also directed to antibodies against the disclosed PACAP peptide immunogen constructs. In particular, the PACAP peptide immunogen constructs of the present disclosure are able to stimulate the generation of highly specific functional antibodies that are cross-reactive with the full-length PACAP molecule. The disclosed antibodies bind with high specificity to PACAP without much, if any, directed to the heterologous Th epitopes employed for immunogenicity enhancement, which is in sharp contrast to antibodies produced using conventional KLH or toxoid proteins or other biological carriers used for such peptide immunogenicity enhancement. Thus, the disclosed PACAP peptide immunogen constructs are capable of breaking the immune tolerance against self-PACAP, with a high responder rate, compared to other peptide or protein immunogens. Based on their unique characteristics and properties, the disclosed antibodies elicited by the PACAP peptide immunogen constructs are capable of providing a prophylactic and immunotherapeutic approach to treating patients suffering from PACAP mediated disorders, including pain, headache, and migraine.

In some embodiments, the disclosed antibodies are directed against either the N-terminal region of PACAP responsible for downstream cell activation events or against the central and/or C-terminal regions of the PACAP involved in receptor binding (e.g., SEQ ID NOs: 2-20). The highly specific antibodies elicited by the PACAP peptide immunogen constructs can inhibit (1) downstream activation events or (2) PACAP and PAC1 binding, resulting in the suppression of the rise of cellular cAMP. Based on their unique characteristics and properties, the disclosed antibodies elicited by the PACAP peptide immunogen constructs are capable of providing a prophylactic immunotherapeutic approach to treating patients suffering from pain, including headache and migraine.

In a further aspect, the present invention provides human monoclonal antibodies against PACAP induced by patients receiving compositions containing PACAP peptide immunogen constructs of this disclosure. An efficient method to make human monoclonal antibodies from B cells isolated from the blood of a human patient is described by Traggiai, E., et al, 2004, which is incorporated by reference.

The present disclosure is also directed to methods of making and using the disclosed PACAP peptide immunogen constructs, compositions, and antibodies. The disclosed methods provide for the low-cost manufacture and quality control of PACAP peptide immunogen constructs and compositions containing the constructs. The disclosed methods are also directed to preventing and/or treating subjects predisposed to, or suffering from PACAP mediated disorders, including pain, headache, and migraine, using the disclosed PACAP peptide immunogen constructs and/or antibodies elicited from the PACAP peptide immunogen constructs. The disclosed methods also include dosing regimens, dosage forms, and routes for administering the PACAP peptide immunogen constructs to prevent and/or treat PACAP mediated disorders, including pain, headache, and migraine.

### BRIEF DESCRIPTION OF THE DRAWINGS

FIG. 1 is a schematic that depicts the structural features of human PACAP precursor termed prepro-PACAP. PACAP38 and PACAP27 are produced by alternative processing from prepro-PACAP. Prepro-PACAP has 176 amino acids and is initially metabolized by signal proteases to generate a signal peptide (aa 1-25) and a pro-PACAP (aa 26-176). Pro-PACAP is metabolized by pro-hormone convertases and carboxy peptidases to produce a small fragment (aa 26-79), a large PACAP-Related Peptide (PRP) (aa 82-129), the physiological function of which remains unclear, and C-terminal peptides (aa 132-170 and 132-159). The C-terminal peptides are metabolized by peptidylglycine alpha-amidating monooxygenase (PAM) enzymes to form PACAP38 and PACAP27, respectively, which have amidated C-terminals.

FIG. 2 depicts sequence alignments of PACAP, VIP, and Secretin proteins in different animals. Human, rat, mouse and sheep all share identical PACAP38 sequence. The immunogenicity studies conducted in the guinea pigs described in the Examples use the human/rat/mouse/sheep PACAP38 sequence as the base for design exploration of peptide immunogenic constructs.

FIG. 3 depicts the pathway from discovery to commercialization of high precision PACAP designer peptide immunogen constructs and formulations thereof for the treatment of pain, including headache and migraine.

FIG. 4 depicts a comprehensive immunogen design and epitope mapping employing PACAP B cell epitopes ranging in size from 9 to 22 amino acids with their respective SEQ ID NOs.

FIG. 5 depicts immunogenicity studies of PACAP peptide immunogen constructs (SEQ ID NOs: 110 to 131) in guinea pigs (n=3 per group) employing a standard formulation comprising ISA51VG as the adjuvant. The respective PACAP peptide immunogen construct was administered intramuscularly at 0, 3, 6, 9, and 12 weeks post initial immunization (wpi). The immune sera collected at the specified time point were tested by an ELISA using the full-length PACAP38 as the plate coating antigen. The ELISA titers were expressed in Log₁₀. Average titer per group is shown in a short horizontal bar with dots shown for the respective titer of each animal.

FIG. 6 depicts immunogenicity studies of PACAP peptide immunogen constructs (SEQ ID NOs: 110 to 131) in guinea pigs (n=3 per group) employing a standard formulation comprising ISA51VG as the adjuvant. The respective PACAP peptide immunogen construct was administered intramuscularly at 0, 3, 6, 9, and 12 weeks post initial immunization (wpi). The immune sera collected at the specified time point were tested by an ELISA using full-length recombinant PACAP38 protein as the plate coating antigen. The ELISA titers were expressed in Log₁₀. Average titer per group is shown in bar graph for each of the blood collections (3, 6, 9, and 12 wpi).

FIG. 7 depicts functional studies of PACAP peptide immunogen constructs (SEQ ID NOs: 110 to 131) in guinea pigs (n=3 per group) employing a standard formulation comprising ISA51VG as the adjuvant. The respective PACAP peptide immunogen construct was administered intramuscularly at 0, 3, 6, 9 and 12 weeks post initial immunization (wpi). Purified antibodies from immune sera collected from 12 wpi for each of the animals were tested in a neutralization assay. The cAMP level was measured using media alone (control) and for the purified antibodies from each group of pooled immune sera collected from guinea pigs immunized with the respective PACAP peptide immunogen constructs. The % cAMP level was measured against the control cAMP level for assessment of the potency of the corresponding purified antibodies in a dose dependent fashion (from 0, 3.9, 15.6, 62.5, and 250 μg/mL) for each of the representative PACAP peptide immunogen constructs.

FIG. 8 depicts the neutralization activities (expressed as IC₅₀ in μg or nM/mL) of purified antibodies from guinea pig immune sera of selected PACAP peptide immunogen constructs (SEQ ID NOs: 112, 127, 128, 115, and 119).

FIG. 9 depicts the ranking of immunogenicity of representative PACAP peptide immunogen constructs based on PACAP peptide binding profiles and the ability of the purified antibodies from guinea pig immune sera to neutralize cAMP level.

FIGS. 10A-10B depicts the capsaicin induced dermal blood flow (DBF) immunization and challenge regimen and results obtained therefrom. FIG. 10A is a schematic illustrating the immunization dosing regimen of female Balb/c mice using formulations containing a placebo (negative control) or peptide immunogens SEQ ID NOs: 112, 127, or 114, together with a capsaicin-induced challenge regimen. FIG. 10B are graphs illustrating that immunization with PACAP38 peptide immunogens inhibited capsaicin-induced ear dermal blood flow at 6, 9, 12, and 15 weeks post initial immunization (wpi).

### DETAILED DESCRIPTION OF THE INVENTION

The present disclosure is directed to portions of the Pituitary Adenylate Cyclase-Activating Peptide (PACAP) that can be used as B cell epitopes. The present disclosure is also directed to peptide immunogen constructs containing B cell epitopes from PACAP, compositions containing the peptide immunogen constructs, methods of making and using the peptide immunogen constructs, and antibodies produced by the peptide immunogen constructs.

One aspect of the present disclosure is directed B cell epitopes from different portions of PACAP from various organisms. The unstructured PACAP binds non-specifically to the cell membrane, thereby favoring the formation of a stable helical conformation at the central and C-terminal segments of the peptide. The-helix segment of PACAP interacts specifically with the N-terminal domain of the PAC1 receptor, positioning the N-terminal disordered domain of PACAP in the vicinity of the juxtamembrane domain of the receptor. The disclosed functional B cell epitope peptides have between about 9 to about 22 amino acids from the human/rat/mouse PACAP38 peptide (i.e., SEQ ID NO: 1). In certain embodiments, the functional B cell epitope peptides are located at either the N-terminal to central or C-terminal region of the PACAP molecule (e.g., SEQ ID NOs: 2-20, as shown in Table 1) where the N-terminal segment could adopt a specific conformation to activate the PAC1 receptor.

The disclosed B cell epitope peptides derived from PACAP can be linked through an optional heterologous spacer to a heterologous T helper cell (Th) epitope peptide to form a peptide immunogen construct. In certain embodiments, the heterologous spacer is any molecule or chemical structure capable of linking two amino acids and/or peptides together, which can include a chemical compound, a naturally occurring amino acid, a non-naturally occurring amino acid, or any combination thereof. The heterologous Th epitope can be any Th epitope that is capable of enhancing the immune response to the B cell epitope. In certain embodiments, the Th epitope is derived from pathogen proteins having the amino acid sequences of SEQ ID NOs: 70-109 and 160-171, as shown in Table 2.

In certain embodiments, the heterologous Th epitopes employed to enhance the PACAP B cell epitope peptide are derived from natural pathogens EBV BPLF1 (SEQ ID NO: 108), EBV CP (SEQ ID NO: 105), Clostridium tetani (SEQ ID NOs: 70, 73, 100, and 102-104), Cholera Toxin (SEQ ID NO: 77), and Schistosoma mansoni (SEQ ID NO: 76), as well as those idealized artificial Th epitopes derived from Measles Virus Fusion protein (MVF 1 to 5) and Hepatitis B Surface Antigen (HBsAg 1 to 3) in the form of either single sequence or combinatorial sequences (e.g. SEQ ID NOs: 71, 78-95, and 160-171).

The disclosed peptide immunogen constructs contain the PACAP B cell epitope peptide covalently linked, at either the N- or C-terminus through an optional heterologous spacer to the heterologous Th epitope. The disclosed peptide immunogen constructs, containing the B cell epitope and Th epitope, have 20 or more total amino acids. In certain embodiments, the peptide immunogen constructs have the amino acid sequences of SEQ ID NOs: 110-159, as shown in Table 3.

The disclosed PACAP peptide immunogen constructs, containing both designed B cell- and Th-epitope peptides, act together to stimulate the generation of highly specific antibodies directed against PACAP functional sites, including the PACAP receptor binding region located at the central or C-terminus of PACAP or the receptor activation region located at the N-terminus. These antibodies offer therapeutic immune responses to patients predisposed to, or suffering from, pain, including headache and migraine.

Another aspect of the present disclosure is directed to peptide compositions, including pharmaceutical compositions, containing a PACAP peptide immunogen construct. The compositions can contain one or more PACAP peptide immunogen construct, pharmaceutically acceptable delivery carriers, adjuvants, and/or be formulated into a stabilized immunostimulatory complex using a CpG oligomer. In certain embodiments, a mixture of PACAP peptide immunogen constructs have heterologous Th epitopes derived from different pathogens that can be used to allow coverage of as broad a genetic background in patients leading to a higher percentage in responder rate upon immunization for the prevention and/or treatment of patients with PACAP mediated disorders, including pain, headache, and migraine.

Synergistic enhancement in PACAP immunogen constructs can be observed in the peptide compositions of this disclosure. The antibody response derived from the administration of such compositions containing PACAP peptide immunogen constructs was mostly (>90%) focused on the desired cross-reactivity against the PACAP functional site(s) or receptor binding region peptides of the B cell epitope(s) without much, if any, directed to the heterologous Th epitopes employed for immunogenicity enhancement. This is in sharp contrast to standard methods that use a conventional carrier protein, such as KLH, toxoid, or other biological carriers used for such peptide antigenicity enhancement.

The present disclosure is also directed to pharmaceutical compositions and formulations for the prevention and/or treatment of migraine. In some embodiments, pharmaceutical compositions comprising a stabilized immunostimulatory complex, which is formed by mixing a CpG oligomer with a peptide composition containing a mixture of PACAP peptide immunogen constructs through electrostatic association, to further enhance the PACAP peptide immunogenicity towards the desired cross-reactivity with the full-length PACAP38 peptide (e.g., SEQ ID NO:1).

In other embodiments, pharmaceutical compositions comprising the disclosed PACAP peptide immunogen construct, or mixture of constructs, are formulated with pharmaceutically acceptable delivery vehicles or adjuvants, such as mineral salts, including Alum gel (ALHYDROGEL) or Aluminum phosphate (ADJU-PHOS) to form a suspension formulation, or with MONTANIDE™ ISA 51 or 720 as adjuvant to form water-in-oil emulsions, that can be used for the prevention and/or treatment of pain, including headache and migraine.

The present disclosure is also directed to antibodies directed against the disclosed PACAP peptide immunogen constructs. In particular, the PACAP peptide immunogen constructs of the present disclosure are able to stimulate the generation of highly specific functional antibodies that are cross-reactive with the full-length PACAP molecule. The disclosed antibodies bind with high specificity to PACAP without much, if any, directed to the heterologous Th epitopes employed for immunogenicity enhancement, which is in sharp contrast to antibodies produced using conventional proteins or other biological carriers used for such peptide immunogenicity enhancement. Thus, the disclosed PACAP peptide immunogen constructs are capable of breaking the immune tolerance against self-PACAP, with a high responder rate, compared to other peptide or protein immunogens.

In some embodiments, the disclosed antibodies are directed against either the N-terminal region of PACAP responsible for downstream cell activation events or against the central and/or C-terminal regions of the PACAP involved in receptor binding (e.g., SEQ ID NOs: 2-20). The highly specific antibodies elicited by the PACAP peptide immunogen constructs can inhibit (1) downstream activation events or (2) PACAP and PAC1 binding, resulting in the suppression of the rise of cellular cAMP. Based on their unique characteristics and properties, the disclosed antibodies elicited by the PACAP peptide immunogen constructs are capable of providing a prophylactic immunotherapeutic approach to treating patients suffering from pain, including headache and migraine.

Based on their unique characteristics and properties, the disclosed antibodies elicited by the PACAP peptide immunogen constructs are capable of providing a prophylactic immunotherapeutic approach to treating patients suffering from pain, including headache and migraine.

In a further aspect, the present invention provides human monoclonal antibodies against PACAP induced by patients receiving compositions containing PACAP peptide immunogen constructs of this disclosure. An efficient method to make human monoclonal antibodies from B cells isolated from the blood of a human patient is described by Traggiai, E., et al., 2004, which is incorporated by reference.

The present disclosure is also directed to methods of making the disclosed PACAP peptide immunogen constructs, compositions, and antibodies. The disclosed methods provide for the low-cost manufacture and quality control of PACAP peptide immunogen constructs and compositions containing the constructs, which can be used in methods for treating patients suffering from pain, including headache and migraine.

The present disclosure also includes methods for preventing and/or treating subjects predisposed to, or suffering from, pain, including headache and migraine using the disclosed PACAP peptide immunogen constructs and/or antibodies directed against the PACAP peptide immunogen constructs. The methods for preventing and/or treating migraine in a subject include administering to the subject a composition containing a disclosed PACAP peptide immunogen construct or mixture of constructs. In certain embodiments, the compositions utilized in the methods contain a disclosed PACAP peptide immunogen construct in the form of a stable immunostimulatory complex with negatively charged oligonucleotides, such as CpG oligomers, through electrostatic association, which can be further supplemented with an adjuvant, for administration to patients suffering from pain, including headache and migraine.

The disclosed methods also include dosing regimens, dosage forms, and routes for administering the PACAP peptide immunogen constructs to prevent and/or treat pain including headache and migraine in a subject.

### General

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence, the phrase “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples disclosed herein are illustrative only and not intended to be limiting.

### PACAP Peptide Immunogen Construct

The present disclosure provides peptide immunogen constructs containing a B cell epitope peptide having about 9 to about 22 amino acids with an amino acid sequence from human/rat/mouse/sheep PACAP38 (SEQ ID NO: 1) or from different organisms. In certain embodiments, the B cell epitope peptide has an amino acid sequence selected from SEQ ID NOs: 2-20, shown in Table 1.

The B cell epitope can be covalently linked to a heterologous T helper cell (Th) epitope derived from a pathogen protein (e.g., SEQ ID NOs: 13-64 and 160-171, as shown in Table 2) directly or through an optional heterologous spacer. These constructs, containing both designed B cell- and Th-epitopes act together to stimulate the generation of highly specific antibodies that are cross-reactive with full-length human PACAP38 (SEQ ID NO: 1).

The phrase “PACAP peptide immunogen construct” or “peptide immunogen construct”, as used herein, refers to a peptide with more than about 20 amino acids containing (a) a B cell epitope having more than about 9 contiguous amino acid residues from the full-length PACAP38 peptide (SEQ ID NO: 1); (b) a heterologous Th epitope; and (c) an optional heterologous spacer.

In certain embodiments, the PACAP peptide immunogen construct can be represented by the formulae:

(Th)ₘ-(A)ₙ-(PACAP functional B cell epitope peptide)-X

or

(PACAP functional B cell epitope peptide)-(A)ₙ-(Th)ₘ-X

or

(Th)ₘ-(A)ₙ-(PACAP functional B cell epitope peptide)-(A)ₙ-(Th)ₘ-X

wherein

Th is a heterologous T helper epitope;

A is a heterologous spacer;

(PACAP functional B cell epitope peptide) is a B cell epitope peptide having from 7 to 30 amino acid residues from PACAP that are involved in either receptor binding or receptor activation;

X is an α-COOH or α-CONH₂ of an amino acid;

m is from 1 to about 4; and

n is from 0 to about 10.

The PACAP peptide immunogen constructs of the present disclosure were designed and selected based on a number of rationales, including:
i. the PACAP B cell epitope peptide is non-immunogenic on its own to avoid autologous T cell activation;
ii. the PACAP B cell epitope peptide can be rendered immunogenic by using a protein carrier or a potent T helper epitope(s);
iii. when the PACAP B cell epitope peptide is rendered immunogenic and administered to a host, the peptide immunogen construct:
   a. elicits high titer antibodies preferentially directed against the PACAP B cell epitope(s) and not the protein carrier or T helper epitope(s);
   b. breaks immune tolerance in the immunized host and generates highly specific antibodies having cross-reactivity with full-length PACAP38 peptide (SEQ ID NO: 1);
   c. generates highly specific antibodies capable of inhibiting PACAP and PACAP receptor binding and the associated downstream events such as rise in intracellular cAMP production; and
   d. generates highly specific antibodies capable reduction in vivo of capsaicin triggered dermal blood flow.

The disclosed PACAP peptide immunogen constructs and formulations thereof can effectively function as a pharmaceutical composition to prevent and/or treat subjects predisposed to, or suffering from, pain including headache and migraine.

The various components of the disclosed PACAP peptide immunogen construct are described in further detail below.

a. B Cell Epitope Peptide from PACAP

The present disclosure is directed to a novel peptide composition for the generation of high titer antibodies with specificity for the Pituitary Adenylate Cyclase-Activating Peptide (PACAP) protein of multi-species (e.g., SEQ ID NO: 1). The site-specificity of the peptide immunogen constructs minimizes the generation of antibodies that are directed to irrelevant sites on other regions of PACAP or irrelevant sites on carrier proteins, thus providing a high safety factor.

The amino acid sequences of the PACAP B cell epitopes used in the present disclosure are shown in Table 1 (SEQ ID NOs: 2-66). Human PACAP is encoded by the ADCYAP1 gene. PACAP is similar to vasoactive intestinal peptide (VIP) and binds to the VIP receptor as well as the PACAP receptor (PAC1). Mediated by adenylate cyclase-activating polypeptide 1 receptors, PACAP stimulates adenylate cyclase and subsequently increases the cAMP level in target cells. PACAP is a hypophysiotropic hormone (i.e., a substance that induces activity in the hypophysis) and also functions as a neurotransmitter and neuromodulator. In addition, it plays a role in paracrine and autocrine regulation of certain types of cells.

Research has shown that administration of intravenous PACAP38 triggers delayed “migraine-like headaches” in most subjects who experience migraine headaches. PACAP38-induced migraines are associated with photophobia, phonophobia, nausea, and respond to medication and triptans. Plasma levels of PACAP38 are elevated during migraine attacks (ictal phase), as compared to the baseline levels when patients are pain-free (inter-ictal phase). In patients with migraine attacks, plasma PACAP levels decrease after treatment with sumatriptan, correlating with symptom amelioration. It is currently unclear whether PACAP38 and CGRP mediate overlapping or complimentary pathways. Studies have shown that PACAP38 infusion induces unique delayed migraine-like attacks associated with prolonged facial flushing. In contrast, the onset of CGRP-induce migraine-like attacks is almost immediate.

The unstructured PACAP binds non-specifically to the cell membrane, thereby favoring the formation of a stable helical conformation at the central and C-terminal segments of the peptide. The-helix segment of PACAP interacts specifically with the N-terminal domain of the PAC1 receptor, positioning the N-terminal disordered domain of PACAP in the vicinity of the juxtamembrane domain of the receptor. The N-terminal segment could adopt a specific conformation to activate the PAC1 receptor.

One aspect of the present disclosure is to prevent and/or treat PACAP-mediated disorders, including migraine headaches, with an active immunotherapy that targets PACAP to exert long-term PACAP blockade and clinical efficacy. Thus, the present disclosure is directed to peptide immunogen constructs targeting portions of the full-length PACAP protein (SEQ ID NO: 1) and formulations thereof for prevention and/or treatment of PACAP-mediated disorders.

The B cell epitope portion of the PACAP peptide immunogen construct can contain between about 9 to about 22 amino acids from any portion of the full-length PACAP38 protein represented by SEQ ID NO: 1. In certain embodiments, the B cell epitope peptide, screened and selected based on design rationales, have an amino acid sequence of SEQ ID NOs: 2-66, as shown in Table 1.

In some embodiments, the B cell epitope peptide is from the PAC1 binding region located at the central/C-terminal region of the PACAP38 molecule (e.g., SEQ ID NOs: 7-20). In other embodiments, the B cell epitope peptide is from the PACAP receptor activation region around the N-terminal region of PACAP38 (e.g., SEQ ID NOs: 2-6).

The PACAP B cell epitope peptide of the present disclosure also includes immunologically functional analogues or homologues of PACAP38, including PACAP38 sequences from different organisms. Functional immunological analogues or homologues of PACAP B cell epitope peptide include variants that retain substantially the same immunogenicity as the original peptide. Immunologically functional analogues can have a conservative substitution in an amino acid position; a change in overall charge; a covalent attachment to another moiety; or amino acid additions, insertions, or deletions; and/or any combination thereof.

Antibodies generated from peptide immunogen constructs containing these B cell epitopes from PACAP are highly specific and cross-reactive with the full-length PACAP of various species. Based on their unique characteristics and properties, the disclosed antibodies elicited by the PACAP peptide immunogen constructs are capable of providing a prophylactic immunotherapeutic approach to preventing and/or treating pain including headache and migraine.

b. Heterologous T Helper Cell Epitopes (Th Epitopes)

The present disclosure provides peptide immunogen constructs containing a B cell epitope from PACAP covalently linked to a heterologous T helper cell (Th) epitope directly or through an optional heterologous spacer.

The heterologous Th epitope in the peptide immunogen construct enhances the immunogenicity of the PACAP B cell epitope, which facilitates the production of specific high titer antibodies directed against the optimized target PACAP B cell epitope peptide screened and selected based on design rationales.

The term “heterologous”, as used herein, refers to an amino acid sequence that is derived from an amino acid sequence that is not part of, or homologous with, the wild-type sequence of PACAP. Thus, a heterologous Th epitope is a Th epitope derived from an amino acid sequence that is not naturally found in PACAP (i.e., the Th epitope is not autologous to PACAP). Since the Th epitope is heterologous to PACAP, the natural amino acid sequence of PACAP is not extended in either the N-terminal or C-terminal directions when the heterologous Th epitope is covalently linked to the PACAP B cell epitope peptide.

The heterologous Th epitope of the present disclosure can be any Th epitope that does not have an amino acid sequence naturally found in PACAP. The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e. little, if any, of the antibodies generated by the PACAP peptide immunogen constructs will be directed towards the Th epitope, thus allowing a very focused immune response directed to the targeted B cell epitope peptide of the PACAP molecule.

Th epitopes of the present disclosure include, but are not limited to, amino acid sequences derived from foreign pathogens, as exemplified in Table 2 (e.g., SEQ ID NOs: 70-109 and 160-171). In certain embodiments, the heterologous Th epitopes employed to enhance the PACAP B cell epitope peptide are derived from natural pathogens EBV BPLF1 (SEQ ID NO: 99), EBV CP (SEQ ID NO: 105), Clostridium Tetani (SEQ ID NOs: 70, 73, 100), Cholera Toxin (SEQ ID NO: 77), and Schistosoma mansoni (SEQ ID NO: 76), as well as those idealized artificial Th epitopes derived from Measles Virus Fusion protein (MVF 1 to 5) and Hepatitis B Surface Antigen (HBsAg 1 to 3) in the form of either single sequence (e.g., SEQ ID NOs: 71, 78, 82-86, 88-89, 91-92, 94-95, 160-163, 165-166, and 168-171) or combinatorial sequences (e.g., SEQ ID NOs: 81, 87, 90, 93, 164, and 167). The combinatorial idealized artificial Th epitopes contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 81, 87, 90, 93, 164, and 167, which are shown in Table 2. Th epitope peptides of the present invention provide broad reactivity and immunogenicity to animals and patients from genetically diverse populations.

c. Heterologous Spacer

The disclosed PACAP peptide immunogen constructs optionally contain a heterologous spacer that covalently links the PACAP B cell epitope peptide to the heterologous T helper cell (Th) epitope.

As discussed above, the term “heterologous”, refers to an amino acid sequence that is derived from an amino acid sequence that is not part of, or homologous with, the natural type sequence of PACAP. Thus, the natural amino acid sequence of PACAP is not extended in either the N-terminal or C-terminal directions when the heterologous spacer is covalently linked to the PACAP B cell epitope peptide because the spacer is heterologous to the PACAP sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the PACAP peptide immunogen construct. Structurally, the spacer provides a physical separation of the Th epitope from the B cell epitope of the PACAP fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 67), where Xaa is any amino acid, and preferably aspartic acid.

The spacer can also provide functional features to the PACAP peptide immunogen construct. For example, the spacer can be designed to change the overall charge of the PACAP peptide immunogen construct, which can affect the solubility of the peptide immunogen construct. Additionally, changing the overall charge of the PACAP peptide immunogen construct can affect the ability of the peptide immunogen construct to associate with other compounds and reagents. As discussed in further detail below, the PACAP peptide immunogen construct can be formed into a stable immunostimulatory complex with a highly charged oligonucleotide, such as CpG oligomers, through electrostatic association. The overall charge of the PACAP peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, ß-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the PACAP peptide immunogen construct can be covalently linked at either N- or C-terminal end of the Th epitope and the PACAP B cell epitope peptide. In some embodiments, the spacer is covalently linked to the C-terminal end of the Th epitope and to the N-terminal end of the PACAP B cell epitope peptide. In other embodiments, the spacer is covalently linked to the C-terminal end of the PACAP B cell epitope peptide and to the N-terminal end of the Th epitope. In certain embodiments, more than one spacer can be used, for example, when more than one Th epitope is present in the PACAP peptide immunogen construct. When more than one spacer is used, each spacer can be the same as each other or different. Additionally, when more than one Th epitope is present in the PACAP peptide immunogen construct, the Th epitopes can be separated with a spacer, which can be the same as, or different from, the spacer used to separate the Th epitope from the PACAP B cell epitope peptide. There is no limitation in the arrangement of the spacer in relation to the Th epitope or the PACAP B cell epitope peptide.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, ε-N-Lys-Lys-Lys-Lys (SEQ ID NO: 68), or Lys-Lys-Lys-ε-N-Lys (SEQ ID NO: 69).

d. Specific Embodiments of the PACAP Peptide Immunogen Constructs

In certain embodiments, the PACAP peptide immunogen constructs can be represented by the following formulae:

(Th)ₘ-(A)ₙ-(PACAP functional B cell epitope peptide)-X

or

(PACAP functional B cell epitope peptide)-(A)ₙ-(Th)ₘ-X

or

(Th)ₘ-(A)ₙ-(PACAP functional B cell epitope peptide)-(A)ₙ-(Th)ₘ-X

wherein

Th is a heterologous T helper epitope;

A is a heterologous spacer;

(PACAP functional B cell epitope peptide) is a B cell epitope peptide having from 7 to 30 amino acid residues from PACAP that are involved in either receptor binding or receptor activation;

X is an α-COOH or α-CONH₂ of an amino acid;

m is from 1 to about 4; and

n is from 0 to about 10.

The B cell epitope peptide can contain between about 7 to about 30 amino acids from any portion of the full-length PACAP38 protein represented by SEQ ID NO: 1. In some embodiments, the B cell epitope has an amino acid sequence selected from any of SEQ ID NOs: 2-20, shown in Table 1. In certain embodiments, the B cell epitope peptide is from the PACAP receptor binding region located at the central/C-terminal region of the PACAP38 molecule (SEQ ID NOs: 7-20). In other embodiments, the B cell epitope peptide is from the PAC1 activation region around the N-terminal region of PACAP38 (SEQ ID NOs: 2-6).

The heterologous Th epitope in the PACAP peptide immunogen construct has an amino acid sequence selected from any of SEQ ID NOs: 70-109 and 160-171, and combinations thereof, shown in Table 2. In some embodiments, more than one Th epitope is present in the PACAP peptide immunogen construct.

The optional heterologous spacer is selected from any of Lys-, Gly-, Lys-Lys-Lys-, (α, ε-N)Lys, Pro-Pro-Xaa-Pro-Xaa-Pro (SEQ ID NO: 67), ε-N-Lys-Lys-Lys-Lys (SEQ ID NO: 68), Lys-Lys-Lys-ε-N-Lys (SEQ ID NO: 69), and any combination thereof, where Xaa is any amino acid, but preferably aspartic acid. In specific embodiments, the heterologous spacer is ε-N-Lys-Lys-Lys-Lys (SEQ ID NO: 68) or Lys-Lys-Lys-ε-N-Lys (SEQ ID NO: 69).

In certain embodiments, the PACAP peptide immunogen construct has an amino acid sequence selected from any of SEQ ID NOs: 110-159, as shown in Table 3.

The PACAP peptide immunogen constructs comprising Th epitopes are produced simultaneously in a single solid-phase peptide synthesis in tandem with the PACAP fragment. Th epitopes also include immunological analogues of Th epitopes. Immunological Th analogues include immune-enhancing analogues, cross-reactive analogues and segments of any of these Th epitopes that are sufficient to enhance or stimulate an immune response to the PACAP B cell epitope peptide.

The Th epitope in the PACAP peptide immunogen construct can be covalently linked at either N- or C-terminal end of the PACAP B cell epitope peptide. In some embodiments, the Th epitope is covalently linked to the N-terminal end of the PACAP B cell epitope peptide. In other embodiments, the Th epitope is covalently linked to the C-terminal end of the PACAP B cell epitope peptide. In certain embodiments, more than one Th epitope is covalently linked to the PACAP B cell epitope peptide. When more than one Th epitope is linked to the PACAP B cell epitope peptide, each Th epitope can have the same amino acid sequence or different amino acid sequences. In addition, when more than one Th epitope is linked to the PACAP B cell epitope peptide, the Th epitopes can be arranged in any order. For example, the Th epitopes can be consecutively linked to the N-terminal end of the PACAP B cell epitope peptide, or consecutively linked to the C-terminal end of the PACAP B cell epitope peptide, or a Th epitope can be covalently linked to the N-terminal end of the PACAP B cell epitope peptide while a separate Th epitope is covalently linked to the C-terminal end of the PACAPB cell epitope peptide. There is no limitation in the arrangement of the Th epitopes in relation to the PACAP B cell epitope peptide.

In some embodiments, the Th epitope is covalently linked to the PACAP B cell epitope peptide directly. In other embodiments, the Th epitope is covalently linked to the PACAP fragment through a heterologous spacer.

e. Variants, Homologues, and Functional Analogues

Variants and analogues of the above immunogenic peptide constructs that induce and/or cross-react with antibodies to the preferred PACAP B cell epitope peptides can also be used. Analogues, including allelic, species, and induced variants, typically differ from naturally occurring peptides at one, two, or a few positions, often by virtue of conservative substitutions. Analogues typically exhibit at least 75%, 80%, 85%, 90%, or 95% sequence identity with natural peptides. Some analogues also include unnatural amino acids or modifications of N- or C-terminal amino acids at one, two, or a few positions.

Variants that are functional analogues can have a conservative substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Functional immunological analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the PACAP B cell epitope peptide. Table 2 identifies another variation of a functional analogue for Th epitope peptide. In particular, SEQ ID NOs: 71 and 78 of MvF1 and MvF2 Th are functional analogues of SEQ ID NOs: 88-90 and 94 of MvF4 and MvF5, respectively, in that they differ in the amino acid frame by the deletion (SEQ ID NOs: 71 and 78) or the inclusion (SEQ ID NOs: 88-90 and 94) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 71, 78, 79-81, 88-90, 94, and 160-168) and from Hepatitis Surface protein HBsAg 1-3 Ths (SEQ ID NOs: 82-87, 91-93, 95, and 169-171).

In other embodiments, the heterologous Th epitopes employed to enhance the PACAP B cell epitope peptide are derived from natural pathogens EBV BPLF1 (SEQ ID NO: 99), EBV CP (SEQ ID NO: 105), Clostridium Tetani (SEQ ID NOs: 70, 73, 100), Cholera Toxin (SEQ ID NO: 77), and Schistosoma mansoni (SEQ ID NO: 76).

### Compositions

The present disclosure also provides compositions comprising the disclosed PACAP immunogen peptide constructs.

a. Peptide Compositions

Compositions containing the disclosed PACAP peptide immunogen constructs can be in liquid or solid/lyophilized form. Liquid compositions can include water, buffers, solvents, salts, and/or any other acceptable reagent that does not alter the structural or functional properties of the PACAP peptide immunogen constructs. Peptide compositions can contain one or more of the disclosed PACAP peptide immunogen constructs.

b. Pharmaceutical Compositions

The present disclosure is also directed to pharmaceutical compositions containing the disclosed PACAP peptide immunogen constructs.

Pharmaceutical compositions can contain carriers and/or other additives in a pharmaceutically acceptable delivery system. Accordingly, pharmaceutical compositions can contain a pharmaceutically effective amount of an PACAP peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the PACAP peptide immunogen constructs without having any specific antigenic effect itself. Adjuvants used in the pharmaceutical composition can include oils, oil emulsions, aluminum salts, calcium salts, immune stimulating complexes, bacterial and viral derivatives, virosomes, carbohydrates, cytokines, polymeric microparticles. In certain embodiments, the adjuvant can be selected from alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, EMULSIGEN®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, algammulin, glucans, dextrans, glucomannans, galactomannans, levans, xylans, dimethyldioctadecylammonium bromide (DDA), as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e. w/o/w) emulsion with EmulsIL-6n or EmulsIL-6n D as the adjuvant.

Pharmaceutical compositions can also include pharmaceutically acceptable additives or excipients. For example, pharmaceutical compositions can contain antioxidants, binders, buffers, bulking agents, carriers, chelating agents, coloring agents, diluents, disintegrants, emulsifying agents, fillers, gelling agents, pH buffering agents, preservatives, solubilizing agents, stabilizers, and the like.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the PACAP peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can also be formulated in a suitable dosage unit form. In some embodiments, the pharmaceutical composition contains from about 0.1 μg to about 1 mg of the PACAP peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one PACAP peptide immunogen construct. A pharmaceutical composition containing a mixture of more than one PACAP peptide immunogen construct to allow for synergistic enhancement of the immunoefficacy of the constructs. Pharmaceutical compositions containing more than one PACAP peptide immunogen construct can be more effective in a larger genetic population due to a broad MHC class II coverage thus provide an improved immune response to the PACAP peptide immunogen constructs.

In some embodiments, the pharmaceutical composition contains a PACAP peptide immunogen construct selected from SEQ ID NOs: 110-159 (Table 3), as well as homologues, analogues and/or combinations thereof.

In certain embodiments, PACAP peptide immunogen constructs (SEQ ID NOs: 141-144) with heterologous Th epitopes derived from MVF and HBsAg in a combinatorial form (SEQ ID NOs: 81, 87, 90, and 93, respectively) were mixed in an equimolar ratio for use in a formulation to allow for maximal coverage of a host population having a diverse genetic background.

Furthermore, the antibody response elicited by the PACAP peptide immunogen constructs (e.g., utilizing UBITh®1; SEQ ID NO: 94) were mostly (>90%) focused on the desired cross-reactivity against the B cell epitope peptide of PACAP without much, if any, directed to the heterologous Th epitopes employed for immunogenicity enhancement (Example 6, Table 7). This is in sharp contrast to the conventional protein such as KLH or other biological protein carriers used for such PACAP peptide immunogenicity enhancement.

In other embodiments, pharmaceutical compositions comprising a peptide composition of for example a mixture of the PACAP peptide immunogen constructs in contact with mineral salts including Alum gel (ALHYDROGEL) or Aluminum phosphate (ADJUPHOS) as adjuvant to form a suspension formulation was used for administration to hosts.

Pharmaceutical compositions containing a PACAP peptide immunogen construct can be used to elicit an immune response and produce antibodies in a host upon administration.

c. Immunostimulatory Complexes

The present disclosure is also directed to pharmaceutical compositions containing an PACAP peptide immunogen construct in the form of an immunostimulatory complex with a CpG oligonucleotide. Such immunostimulatory complexes are specifically adapted to act as an adjuvant and/or as a peptide immunogen stabilizer. The immunostimulatory complexes are in the form of a particulate, which can efficiently present the PACAP peptide immunogen to the cells of the immune system to produce an immune response. The immunostimulatory complexes may be formulated as a suspension for parenteral administration. The immunostimulatory complexes may also be formulated in the form of water in oil (w/o) emulsions, as a suspension in combination with a mineral salt or with an in-situ gelling polymer for the efficient delivery of the PACAP peptide immunogen construct to the cells of the immune system of a host following parenteral administration.

The stabilized immunostimulatory complex can be formed by complexing an PACAP peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the PACAP peptide immunogen construct is designed to contain a cationic portion that is positively charged at a pH in the range of 5.0 to 8.0. The net charge on the cationic portion of the PACAP peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the PACAP peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the PACAP peptide immunogen construct is the heterologous spacer. In certain embodiments, the cationic portion of the PACAP peptide immunogen construct has a charge of +4 when the spacer sequence is (α, ε-N)Lys, (α,ε-N)-Lys-Lys-Lys-Lys (SEQ ID NO: 68), or Lys-Lys-Lys-ε-N-Lys (SEQ ID NO: 69).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T. In specific embodiments, the CpG oligonucleotide has the sequence of CpG1:5′ TCg TCg TTT TgT CgT TTT gTC gTT TTg TCg TT 3′ (fully phosphorothioated) (SEQ ID NO: 172), CpG2: 5′ Phosphate TCg TCg TTT TgT CgT TTT gTC gTT 3′ (fully phosphorothioated) (SEQ ID NO: 173), or CpG3 5′ TCg TCg TTT TgT CgT TTT gTC gTT 3′ (fully phosphorothioated) (SEQ ID NO: 174).

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

The present disclosure is also directed to pharmaceutical compositions, including formulations, for the prevention and/or treatment of migraine. In some embodiments, pharmaceutical compositions comprising a stabilized immunostimulatory complex, which is formed through mixing a CpG oligomer with a peptide composition containing a mixture of the PACAP peptide immunogen constructs (e.g., SEQ ID NOs: 110-159) through electrostatic association, to further enhance the immunogenicity of the PACAP peptide immunogen constructs and elicit antibodies that are cross-reactive with the full-length PACAP38 peptide of SEQ ID NO: 1 that are directed at the PAC1 binding or receptor activation regions.

In yet other embodiments, pharmaceutical compositions contain a mixture of the PACAP peptide immunogen constructs (e.g., any combination of SEQ ID NOs: 110-159) in the form of a stabilized immunostimulatory complex with CpG oligomers that are, optionally, mixed with mineral salts, including Alum gel (ALHYDROGEL) or Aluminum phosphate (ADJUPHOS) as an adjuvant with high safety factor, to form a suspension formulation for administration to hosts.

### Antibodies

The present disclosure also provides antibodies elicited by the PACAP peptide immunogen constructs.

The present disclosure provides PACAP peptide immunogen constructs and formulations thereof, cost effective in manufacturing, optimal in their design that are capable of eliciting high titer antibodies targeting the PAC1 binding region (e.g., SEQ ID NOs: 7-20) or receptor activation region (SEQ ID NOs: 2-6) of the PACAP38 molecule that is capable of breaking the immune tolerance against self-protein PACAP with a high responder rate in immunized hosts. The antibodies generated by the PACAP peptide immunogen constructs have high affinity towards the PAC1 binding or receptor activation regions.

In some embodiments, PACAP peptide immunogen constructs for eliciting antibodies comprise a hybrid of a PACAP peptide targeting the PAC1 binding region or receptor activation region of the PACAP38 molecule (e.g., SEQ ID NOs: 7-20 and 2-6, respectively) linked to a heterologous Th epitope derived from pathogenic proteins such as Measles Virus Fusion (MVF) protein and others (SEQ ID NOs: 70-109 and 160-171) through an optional spacer. The B cell epitope and Th epitope peptide of the PACAP peptide immunogen constructs act together to stimulate the generation of highly specific antibodies cross-reactive with the PACAP receptor binding or activation region of the PACAP38 molecule (SEQ ID NO: 1).

Traditional methods for immunopotentiating a peptide, such as through chemical coupling to a carrier protein, for example, Keyhole Limpet Hemocyanin (KLH) or other carrier proteins such as Diphtheria toxoid (DT) and Tetanus Toxoid (TT) proteins, typically result in the generation of a large amount of antibodies directed against the carrier protein. Thus, a major deficiency of such peptide-carrier protein compositions is that most (>90%) of antibodies generated by the immunogen are the non-functional antibodies directed against the carrier protein KLH, DT or TT, which can lead to epitopic suppression.

Unlike the traditional method for immunopotentiating a peptide, the antibodies generated by the disclosed PACAP peptide immunogen constructs (e.g. SEQ ID NOs: 110-159) bind with highly specificity to the PACAP B cell epitope peptide (SEQ ID NOs: 2-20) with little, if any, antibodies directed against the heterologous Th epitope (e.g., SEQ ID NOs: 70-109 and 160-171) or optional heterologous spacer.

Based on their unique characteristics and properties, the disclosed antibodies elicited by the PACAP peptide immunogen constructs are capable of providing a prophylactic immunotherapeutic approach to preventing and/or treating PACAP mediated disorders, including pain, headache, and migraine.

### Methods

The present disclosure is also directed to methods for making and using the PACAP peptide immunogen constructs, compositions, and pharmaceutical compositions.

a. Methods for Manufacturing the PACAP Peptide Immunogen Construct

The PACAP peptide immunogen constructs of this disclosure can be made by chemical synthesis methods well known to the ordinarily skilled artisan (see, e.g., Fields, G. B., et al., 1992). The PACAP peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of PACAP peptide immunogen constructs comprising combinatorial library peptides for Th epitopes can be accomplished by providing a mixture of alternative amino acids for coupling at a given variable position.

After complete assembly of the desired PACAP peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of PACAP peptide immunogen constructs, immunogenicity, and yield can be assured. A detailed description of the manufacturing of the PACAP peptide immunogen construct through solid phase peptide synthesis is provided in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs.

Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The PACAP peptide immunogen constructs can also be made using recombinant DNA technology including nucleic acid molecules, vectors, and/or host cells. As such, nucleic acid molecules encoding the PACAP peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the PACAP peptide immunogen construct and immunologically functional analogues thereof. For example, methods can include a step of incubating a host cell containing an expression vector containing a nucleic acid molecule encoding an PACAP peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the immunostimulatory complexes comprising PACAP peptide immunogen constructs and CpG oligodeoxynucleotide (ODN) molecule. Stabilized immunostimulatory complexes (ISC) are derived from a cationic portion of the PACAP peptide immunogen construct and a polyanionic CpG ODN molecule. The self-assembling system is driven by electrostatic neutralization of charge. Stoichiometry of the molar charge ratio of cationic portion of the PACAP peptide immunogen construct to anionic oligomer determines extent of association. The non-covalent electrostatic association of PACAP peptide immunogen construct and CpG ODN is a completely reproducible process. The peptide/CpG ODN immunostimulatory complex aggregates, which facilitate presentation to the “professional” antigen presenting cells (APC) of the immune system thus further enhancing the immunogenicity of the complexes. These complexes are easily characterized for quality control during manufacturing. The peptide/CpG ISC are well tolerated in vivo. This novel particulate system comprising CpG ODN and PACAP peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen presenting cells (APC). Consequently, such formulations are novel conceptually and offer potential advantages by promoting the stimulation of immune responses by alternative mechanisms.

c. Methods for the Manufacturing of Pharmaceutical Compositions

Various exemplary embodiments also encompass pharmaceutical compositions containing PACAP peptide immunogen constructs. In certain embodiments, the pharmaceutical compositions employ water in oil emulsions and in suspension with mineral salts.

In order for a pharmaceutical composition to be used by a large population, safety becomes another important factor for consideration. Despite there has been use of water-in-oil emulsions in many clinical trials, Alum remains the major adjuvant for use in formulations due to its safety. Alum or its mineral salts Aluminum phosphate (ADJUPHOS) are, therefore, frequently used as adjuvants in preparation for clinical applications.

Other adjuvants and immunostimulating agents include 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethyl amine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include MF59 (see WO 1990/014837 to Van Nest, G., et al., which is hereby incorporated by reference in its entirety), containing 5% Squalene, 0.5% TWEEN 80, and 0.5% Span 85 (optionally containing various amounts of MTP-PE) formulated into submicron particles using a microfluidizer; SAF, containing 10% Squalene, 0.4% TWEEN 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) containing 2% squalene, 0.2% TWEEN 80, and one or more bacterial cell wall components selected from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins (IL-1, IL-2, and IL-12), macrophage colony stimulating factor (M-CSF), and tumor necrosis factor (TNF-α).

The choice of an adjuvant depends on the stability of the immunogenic formulation containing the adjuvant, the route of administration, the dosing schedule, the efficacy of the adjuvant for the species being immunized, and, in humans, a pharmaceutically acceptable adjuvant is one that has been approved or is approvable for human administration by pertinent regulatory bodies. For example, alum, MPL or Incomplete Freund's adjuvant (Chang, J. C. C., et al., 1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

d. Methods of Using Pharmaceutical Compositions

The present disclosure also includes methods of using pharmaceutical compositions containing PACAP peptide immunogen constructs.

In certain embodiments, the pharmaceutical compositions containing PACAP peptide immunogen constructs can be used for the treatment of migraine.

In some embodiments, the methods comprise administering a pharmaceutical composition comprising a pharmacologically effective amount of an PACAP peptide immunogen construct to a host in need thereof. In certain embodiments, the methods comprise administering a pharmaceutical composition comprising a pharmacologically effective amount of an PACAP peptide immunogen construct to a warm-blooded animal (e.g., humans, Cynomolgus macaques, mice) to elicit highly specific antibodies cross-reactive with the full-length human/rat/mouse/sheep PACAP38 molecule (SEQ ID NO: 1).

In certain embodiments, the pharmaceutical compositions containing PACAP peptide immunogen constructs can be used to treat migraine as shown in in vivo capsaicin induced dorsal blood flow model.

e. In Vitro Functional Assays and In Vivo Proof of Concept Studies

Antibodies elicited in immunized hosts by the PACAP peptide immunogen constructs can be used in in vitro functional assays. These functional assays include, but are not limited to:

(1) in vitro binding to PACAP protein (SEQ ID NO: 1);

(2) inhibition in vitro of PACAP binding to its receptor;

(3) inhibition in vitro of intracellular cAMP elevation;

(4) inhibition in vivo of capsaicin induced dorsal blood flow model in mice.

### Specific Embodiments

(1) A PACAP peptide immunogen construct having about 20 or more amino acids, represented by the formulae:

(Th)ₘ-(A)ₙ-(PACAP functional B cell epitope peptide)-X

or

(PACAP functional B cell epitope peptide)-(A)ₙ-(Th)ₘ-X

or

(Th)ₘ-(A)ₙ-(PACAP functional B cell epitope peptide)-(A)ₙ-(Th)ₘ-X

wherein

Th is a heterologous T helper epitope;

A is a heterologous spacer;

(PACAP functional B cell epitope peptide) is a B cell epitope peptide having from 9 to about 22 amino acid residues of PACAP (SEQ ID NO: 1);

X is an α-COOH or α-CONH₂ of an amino acid;

m is from 1 to about 4; and

n is from 0 to about 10.

(2) The PACAP peptide immunogen construct according to (1), wherein the PACAP functional B cell epitope peptide is selected from the group consisting of SEQ ID NOs: 2-20.
(3) The PACAP peptide immunogen construct according to (1), wherein the Th epitope is selected from the group consisting of SEQ ID NOs: 70-109 and 106-171.
(4) The PACAP peptide immunogen construct according to (1), wherein the PACAP functional B cell epitope peptide is selected from the group consisting of SEQ ID NOs: 2-20 and the Th epitope is selected from the group consisting of SEQ ID NOs: 70-109 and 106-171.
(5) The PACAP peptide immunogen construct according to (1), wherein the peptide immunogen construct is selected from the group consisting of SEQ ID NOs: 110-159.
(6) An PACAP peptide immunogen construct comprising:
a. a B cell epitope comprising from about 9 to about 22 amino acid residues from the PACAP38 sequence of SEQ ID NO: 1;
b. a T helper epitope comprising an amino acid sequence selected from the group consisting of SEQ ID NOs: 70-109 and 160-171, and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 68), Lys-Lys-Lys-ε-N-Lys (SEQ ID NO: 69), and Pro-Pro-Xaa-Pro-Xaa-Pro (SEQ ID NO: 67), and any combination thereof,
wherein the B cell epitope is covalently linked to the T helper epitope directly or through the optional heterologous spacer.
   (7) The PACAP peptide immunogen construct of (6), wherein the B cell epitope is selected from the group consisting of SEQ ID NOs: 2-20.
   (8) The PACAP peptide immunogen construct of (6), wherein the optional heterologous spacer is (α, ε-N)Lys, ε-N-Lys-Lys-Lys-Lys (SEQ ID NO: 68), Lys-Lys-Lys-ε-N-Lys (SEQ ID NO: 69), or Pro-Pro-Xaa-Pro-Xaa-Pro (SEQ ID NO: 67), where Xaa is any amino acid.
   (9) The PACAP peptide immunogen construct of (6), wherein the T helper epitope is covalently linked to the amino- or carboxyl-terminus of the B cell epitope.
   (10) The PACAP peptide immunogen construct of (6), wherein the T helper epitope is covalently linked to the amino- or carboxyl-terminus of the B cell epitope through the optional heterologous spacer.
   (11) A composition comprising the PACAP peptide immunogen construct according to (1).
   (12) A pharmaceutical composition comprising:
a. a peptide immunogen construct according to (1); and
b. a pharmaceutically acceptable delivery vehicle and/or adjuvant.
   (13) The pharmaceutical composition of (12), wherein
a. the PACAP functional B cell epitope peptide is selected from the group consisting of SEQ ID NO: 2-20;
b. the Th epitope is selected from the group consisting of SEQ ID NOs: 70-109 and 160-171; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 68), Lys-Lys-Lys-ε-N-Lys (SEQ ID NO: 69), and Pro-Pro-Xaa-Pro-Xaa-Pro (SEQ ID NO:67), and any combination thereof; and
wherein the PACAP peptide immunogen construct is mixed with an CpG oligodeoxynucleotide (ODN) to form a stabilized immunostimulatory complex.
   (14) The pharmaceutical composition of (12), wherein
a. the PACAP peptide immunogen construct is selected from the group consisting of SEQ ID NOs: 110-131 and 132-159; and
wherein the PACAP peptide immunogen construct is mixed with an CpG oligodeoxynucleotide (ODN) to form a stabilized immunostimulatory complex.
   (15) A method for generating antibodies against PACAP in an animal comprising administering the pharmaceutical composition according to (12) to the animal.
   (16) An isolated antibody or epitope-binding fragment thereof that specifically binds to the PAC1 binding or activation region of SEQ ID NOs: 2-20.
   (17) The isolated antibody or epitope-binding fragment thereof according to (16) bound to the PACAP peptide immunogen construct.
   (18) A composition comprising the isolated antibody or epitope-binding fragment thereof according to (16).
   (19) A method of preventing and/or treating migraine in an animal comprising administering the pharmaceutical composition of (12) to the animal.

### Example 1

### Synthesis of PACAP Related Peptides and Preparation of Formulations Thereof

a. Synthesis of PACAP Related Peptides

Methods for synthesizing PACAP related peptides that were included in the development effort of PACAP peptide immunogen constructs are described. The peptides were synthesized in small-scale amounts that are useful for serological assays, laboratory pilot and field studies, as well as large-scale (kilogram) amounts, which are useful for industrial/commercial production of pharmaceutical compositions. A large repertoire of PACAP B cell epitope peptides having sequences with lengths from approximately 9 to 38 amino acids were designed for epitope mapping and for the screening and selection of the most optimal peptide immunogen constructs for use in an efficacious PACAP targeted therapeutic vaccine.

Representative full-length PACAP38 from human/mouse/rat (SEQ ID NO: 1) as well as PACAP peptide fragments and 10-mer peptide employed for epitope mapping in various serological assays (SEQ ID NOs: 2-66) are listed in Table 1.

Selected PACAP B cell epitope peptides were made into PACAP peptide immunogen constructs by synthetically linking to a carefully designed helper T cell (Th) epitope peptide derived from pathogen proteins, including Measles Virus Fusion protein (MVF), Hepatitis B Surface Antigen protein (HBsAg), influenza, Clostridum tetani, and Epstein-Barr virus (EBV) identified in Table 2 (SEQ ID NOs: 70-109 and 160-171). The Th epitope peptides were used either in a single sequence (e.g., SEQ ID NOs: 71, 78, 82-86, 88-89, 91-92, 94-95, 160-163, 165-166, and 168-171) or combinatorial sequences (e.g., SEQ ID NOs: 81, 87, 90, 93, 164, and 167) to enhance the immunogenicity of their respective PACAP peptide immunogen constructs.

Representative PACAP peptide immunogen constructs selected from hundreds of peptide constructs are identified in Table 3 (SEQ ID NOs: 110-159). All peptides used for immunogenicity studies or related serological tests for detection and/or measurement of anti-PACAP antibodies were synthesized on a small-scale using F-moc chemistry by peptide synthesizers of Applied BioSystems Models 430A, 431 and/or 433. Each peptide was produced by an independent synthesis on a solid-phase support, with F-moc protection at the N-terminus and side chain protecting groups of trifunctional amino acids. Completed peptides were cleaved from the solid support and side chain protecting groups were removed by 90% Trifluoroacetic acid (TFA). Synthetic peptide preparations were evaluated by Matrix-Assisted Laser Desorption/Ionization-Time-Of-Flight (MALDI-TOF) Mass Spectrometry to ensure correct amino acid content. Each synthetic peptide was also evaluated by Reverse Phase HPLC (RP-HPLC) to confirm the synthesis profile and concentration of the preparation. Despite rigorous control of the synthesis process (including stepwise monitoring the coupling efficiency), peptide analogues were also produced due to unintended events during elongation cycles, including amino acid insertion, deletion, substitution, and premature termination. Thus, synthesized preparations typically included multiple peptide analogues along with the targeted peptide.

Despite the inclusion of such unintended peptide analogues, the resulting synthesized peptide preparations were nevertheless suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides. Large scale peptide syntheses in the multi-hundred to kilo gram quantities can be conducted on a customized automated peptide synthesizer UBI2003 or the like at 15 mmole to 150 mmole scale.

For active ingredients used in the final pharmaceutical composition for clinical trials, PACAP peptide immunogen constructs were purified by preparative RP-HPLC under a shallow elution gradient and characterized by MALDI-TOF mass spectrometry, amino acid analysis and RP-HPLC for purity and identity.

b. Preparation of Compositions Containing PACAP Peptide Immunogen Constructs

Formulations employing water-in-oil emulsions and in suspension with mineral salts were prepared. In order for a pharmaceutical composition designed to be used by a large population, safety becomes another important factor for consideration. Despite the fact that water-in-oil emulsions have been used in humans as pharmaceutical compositions in many clinical trials, Alum remains the major adjuvant for use in pharmaceutical composition due to its safety. Alum or its mineral salts ADJUPHOS (Aluminum phosphate) are therefore frequently used as adjuvants in preparation for clinical applications.

Briefly, the formulations specified in each of the study groups described below generally contained all types of designer PACAP peptide immunogen constructs. Over 40 designer PACAP peptide immunogen constructs were carefully evaluated in guinea pigs for their relative immunogenicity against the corresponding PACAP peptide used as the B cell epitope peptide. Epitope mapping and serological cross-reactivities were analyzed amongst the varying homologous peptides by ELISA assays using plates coated with peptides selected from the list with SEQ ID NOs: 1-66.

The PACAP peptide immunogen constructs at varying amounts were prepared in a water-in-oil emulsion with Seppic MONTANIDE™ ISA 51 as the approved oil for human use, or mixed with mineral salts ADJUPHOS (Aluminum phosphate) or ALHYDROGEL (Alum) as specified. Compositions were typically prepared by dissolving the PACAP peptide immunogen constructs in water at about 20 to 2,000 μg/mL and formulated with MONTANIDE™ ISA 51 into water-in-oil emulsions (1:1 in volume) or with mineral salts ADJUPHOS or ALHYDROGEL (Alum) (1:1 in volume). The compositions were kept at room temperature for about 30 min and mixed by vortex for about 10 to 15 seconds prior to immunization. Animals were immunized with 2 to 3 doses of a specific composition, which were administered at time 0 (prime) and 3 weeks post initial immunization (wpi) (boost), optionally 5 or 6 wpi for a second boost, by intramuscular route. Sera from the immunized animals were then tested with selected B cell epitope peptide(s) to evaluate the immunogenicity of the various PACAP peptide immunogen constructs present in the formulation and for the corresponding sera's cross-reactivity with PACAP proteins. Those PACAP peptide immunogen constructs with potent immunogenicity found in the initial screening in guinea pigs were further tested in in vitro assays for their corresponding sera's functional properties. The selected candidate PACAP peptide immunogen constructs were then prepared in water-in-oil emulsion, mineral salts, and alum-based formulations for dosing regimens over a specified period as dictated by the immunization protocols.

Only the most promising PACAP peptide immunogen constructs were further assessed extensively prior to being incorporated into final formulations for immunogenicity, duration, toxicity and efficacy studies in GLP guided preclinical studies in preparation for submission of an Investigational New Drug application followed by clinical trials in patients suffering from migraine.

The following examples serve to illustrate the present invention and are not to be used to limit the scope of the invention.

### Example 2

### Serological Assays and Reagents

Serological assays and reagents for evaluating functional immunogenicity of the PACAP peptide immunogen constructs and formulations thereof are described in detail below.

a. PACAP or PACAP B Cell Epitope Peptide-Based ELISA Tests for Immunogenicity and Antibody Specificity Analysis

ELISA assays for evaluating immune serum samples described in the following Examples were developed and described below. The wells of 96-well plates were coated individually for 1 hour at 37° C. with 100 μL of PACAP or PACAP B cell epitope peptides (e.g., SEQ ID NOs: 1 to 66), at 2 μg/mL (unless noted otherwise), in 10 mM NaHCO₃ buffer, pH 9.5 (unless noted otherwise).

The PACAP or PACAP B cell epitope peptide-coated wells were incubated with 250 μL of 3% by weight gelatin in PBS at 37° C. for 1 hour to block non-specific protein binding sites, followed by three washes with PBS containing 0.05% by volume TWEEN® 20 and dried. Sera to be analyzed were diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) were added to each of the wells and allowed to react for 60 minutes at 37° C. The wells were then washed six times with 0.05% by volume TWEEN® 20 in PBS in order to remove unbound antibodies. Horseradish peroxidase (HRP)-conjugated species (e.g., guinea pig or rat) specific goat polyclonal anti-IgG antibody or Protein A/G were used as a labeled tracer to bind with the antibody/peptide antigen complex formed in positive wells. One hundred microliters (100 μL) of the HRP-labeled detection reagent, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined. For the determination of antibody titers of the vaccinated animals that received the various peptide vaccine formulations, a 10-fold serial dilutions of sera from 1:100 to 1:10,000 or a 4-fold serial dilutions of sera from 1:100 to 1:4.19×10⁸ were tested, and the titer of a tested serum, expressed as Log₁₀, was calculated by linear regression analysis of the A₄₅₀ with the cutoff A₄₅₀ set at 0.5.

b. Assessment of Antibody Reactivity Towards Th Peptide by Th Peptide-Based ELISA Tests

The wells of 96-well ELISA plates were coated individually for 1 hour at 37° C. with 100 μL of Th peptide at 2 μg/mL (unless noted otherwise), in 10 mM NaHCO₃ buffer, pH 9.5 (unless noted otherwise) in similar ELISA method and performed as described above. For the determination of antibody titers of the vaccinated animals that received the various PACAP peptide vaccine formulations, 10-fold serial dilutions of sera from 1:100 to 1:10,000 were tested, and the titer of a tested serum, expressed as Log₁₀, was calculated by linear regression analysis of the A₄₅₀ with the cutoff A₄₅₀ set at 0.5.

c. Fine Specificity Analyses of a Target PACAP B Cell Epitope Peptide Determined by Epitope mapping through B cell epitope cluster 10-mer peptide-based ELISA tests

Fine specificity analyses of anti-PACAP antibodies from hosts immunized with PACAP peptide immunogen constructs could be determined by epitope mapping using B cell epitope cluster 10mer peptide-based ELISA tests. Briefly, the wells of 96-well plates can be coated with individual PACAP or related 10-mer peptides (SEQ ID NOs: 21-66) at 0.5 μg per 0.1 mL per well and then 100 μL serum samples (1:100 dilution in PBS) can be incubated in 10-mer plate wells in duplicate following the steps of the antibody ELISA method described above. The target B cell epitope specificity analyses of anti-PACAP antibodies from immunized hosts can be tested with corresponding PACAP peptide, or with non-relevant control peptide for specificity confirmation.

d. Immunogenicity Evaluation

Preimmune and immune serum samples from animal or human subjects were collected according to experimental vaccination protocols and heated at 56° C. for 30 minutes to inactivate serum complement factors. Following the administration of the vaccine formulations, blood samples were obtained according to protocols and their immunogenicity against specific target site(s) were evaluated by corresponding PACAP B cell epitope peptide-based ELISA tests. Serially diluted sera were tested and positive titers were expressed as Log₁₀ of the reciprocal dilution. Immunogenicity of a particular vaccine formulation is assessed for its ability to elicit high titer antibody response directed against the desired epitope specificity within the target antigen and high cross-reactivities with PACAP proteins, while maintaining a low to negligible antibody reactivity towards the T helper cell epitopes employed to provide enhancement of the desired B cell responses.

### Example 3

### Assessment of Functional Properties of Antibodies Elicted by the PACAP Peptide Immunnogen Constructs and Formulations Thereof in an In Vitro Assay for Intracellular Camp Production

Immune sera or purified anti-PACAP antibodies in immunized vaccines were further tested for their ability to suppress the PACAP-induced intracellular AMP production.

a. Antibody Purification

All antibody purification procedures were followed according to manual of Protein A Sepharose CL-4B antibody purification resin (GE Healthcare, Life Sciences, Cat no. 17-0963-03). The respective concentrations of IgG purification for each of the groups were carefully calibrated for use in in vitro assay.

b. Cell Preparation and Maintenance

SH-SY5Y cell line was purchased from the American Type Culture Collection (CRL-2266™, Manassas, Va.). The base medium for this cell line is a 1:1 mixture of ATCC-formulated Eagle's Minimum Essential Medium, Catalog No. 30-2003, and F12 Medium. To make the complete growth medium, fetal bovine serum was added to a final concentration of 10%. The cells were maintained in a humidified 37° C. incubator with 5% CO2.

c. cAMP Level Detection

The potency of the respective purified antibodies from 12 wpi immune sera from guinea pigs vaccinated with the representative PACAP peptide immunogen constructs were evaluated for their ability to neutralize PACAP38-induced cAMP production in a cAMP assay. Three hundred microliters (300 μL) of serum from immunized guinea pigs were purified by protein A. These purified antibodies from respective immune sera were incubated with human recombinant PACAP38 protein (5 nM) for 1 hour at 37° C. In an cAMP assay for intracellular cAMP detection, human SH-SY5Y cells were trypsinized and re-suspended at 2×10⁶/mL density, containing 20,000 cells, in 0.3% serum medium containing IBMX. PACAP38 protein/antibody mixtures were transferred respectively to 96-well white polystyrene plate with equal ratio and incubated at room temperature for 30 minutes. Following the cAMP-Glo assay (Promega, cat. no. V1501), 7.5 μL of lysis buffer was added into each of the wells followed by an incubation period of 30 minutes. During the incubation period, Protein Kinase A was freshly prepared in the reaction buffer and 15 μL of PKA buffer was added to each well and incubated for another 20 minutes. Following the reaction, 30 μL of Kinase-Glo buffer was added to each of the wells and incubated for another 10 minutes. After the incubation, the signal from the plate was read by a luminescence reader (SpectraMax i3x Multi-Mode Microplate Reader). Immune sera or purified anti-PACAP38 antibodies from immunized animals were future tested for their ability to suppress the PACAP38-induced intracellular cAMP production.

### Example 4

### Animals Used in Safety, Immunogenicity, Toxicity and Efficacy Studies

a. Guinea Pies

Immunogenicity studies were conducted in mature, naïve, adult male and female Duncan-Hartley guinea pigs (300-350 g/BW). The experiments utilized at least 3 guinea pigs per group. Protocols involving Duncan-Hartley guinea pigs (8-12 weeks of age; Covance Research Laboratories, Denver, Pa., USA) were performed under approved IACUC applications at a contracted animal facility under UBI sponsorship.

b. Immunization of Guinea Pies with PACAP38 Peptide Immunogen Constructs

A total of 66 guinea pigs were used for the immunization and all guinea pigs were divided into 22 groups. Animals in the experimental groups were injected with the respective PACAP peptide immunogen constructs formulated with ISA 51 and CpG at 400 μg/1.0 mL dose for prime and boost immunizations under intramuscular route. A total of five doses were administered at 0, 3, 6, 9, 12, WPI. All animals had free access to rodent chow diet and water. The animals were bled at 0, 3, 6, 9, 12, and 15 WPI. Blood samples were collected for immunogenicity assessment by ELISA for titer determination against PACAP38. Further analysis was conducted by purified antibodies collected from 12 wpi immune sera for their respective ability to inhibit intracellular cAMP production by detecting cAMP level using a cAMP assay as described above. All protocols followed the Principles of Laboratory Animal Care. Blood collection was carried out as indicated in the protocol. Antibody titers were tested for anti-PACAP (mouse) by ELISA assay.

### Example 5

### Vaccine Formulations for Immunogenicity Assessment of PACAP Peptide Constructs in Guinea Pigs

Pharmaceutical compositions and vaccine formulations used in each experiment are described in greater detail as shown below.

Briefly, the formulations specified in each of the study groups generally contained all types of designer PACAP peptide immunogen constructs with a segment of the PACAP B cell epitope peptide linked via different type of spacers (e.g., εLys (εK) or lysine-lysine-lysine (KKK) to enhance the peptide construct's solubility) and promiscuous helper T cell epitopes including two sets of artificial T helper epitopes derived from Measles virus fusion protein and Hepatitis B surface antigen. The PACAP B cell epitope peptides were linked at the N- or C-terminus of the designer peptide constructs. Many designer PACAP peptide immunogen constructs were initially evaluated in guinea pigs for their relative immunogenicity with the corresponding PACAPB cell epitope peptides. The PACAP peptide immunogen constructs were either prepared under varying amounts in a water-in-oil emulsion with Seppic MONTANIDE ISA 51 as the approved oil for human vaccine use, or with mineral salts (ADJUPHOS) or ALHYDROGEL (Alum) as a suspension, as specified. Formulations were usually prepared by dissolving the PACAP peptide constructs in water at about 20 to 800 μg/mL and formulated either with MONTANIDE ISA 51 into water-in-oil emulsions (1:1 in volume) or with mineral salts (ADJUPHOS) or ALHYDROGEL (Alum) (1:1 in volume). The formulations were kept at room temperature for about 30 min and mixed by vortex for about 10 to 15 seconds prior to immunization.

Some animals were immunized with 2 to 5 doses of a specific vaccine formulation, which were administered at time 0 (prime) and 3 weeks post initial immunization (wpi) (boost), optionally 5 or 6 wpi for a second boost, by intramuscular route. These immunized animals were then evaluated for the immunogenicity of the corresponding PACAP peptide immunogen constructs used in the respective formulations for their cross-reactivity with the corresponding PACAP B cell epitope peptides or full-length PACAP. Those PACAP peptide immunogen constructs with potent immunogenicity in the initial screening in guinea pigs can be further tested in both water-in-oil emulsion, mineral salts, and alum-based formulations in other organisms for dosing regimens over a specified period as dictated by the immunization protocols.

Only the most promising PACAP peptide immunogen construct candidates were further assessed extensively to evaluate for their ability to breakout immune tolerance using PACAP peptide immunogen constructs. The PACAP peptide immunogen constructs with best immunogenicity in mice, which elicited anti-PACAP antibody titers against endogenous PACAP; especially for their capability of suppressing capsaicin induced dermal blood flow in mice model. The optimized PACAP peptide immunogen constructs can be incorporated into final vaccine formulations for GLP guided immunogenicity, duration, toxicity and proof of efficacy studies in preparation for submission of an Investigational New Drug application and clinical trials in patients with migraine.

### Example 6

### Design Rationale, Screening, Identification, Assessment of Functional Properties and Optimization of Multi-Component Vaccine Formulations Incorporating PACAP Peptide Immunogen Constructs for Treatment of Migraine

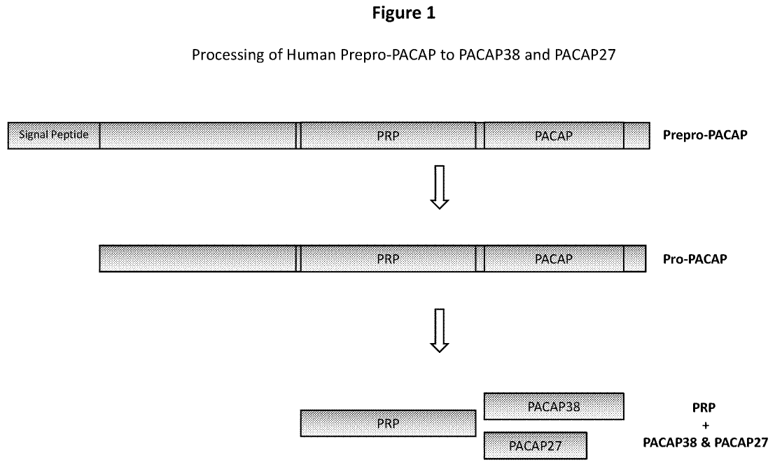

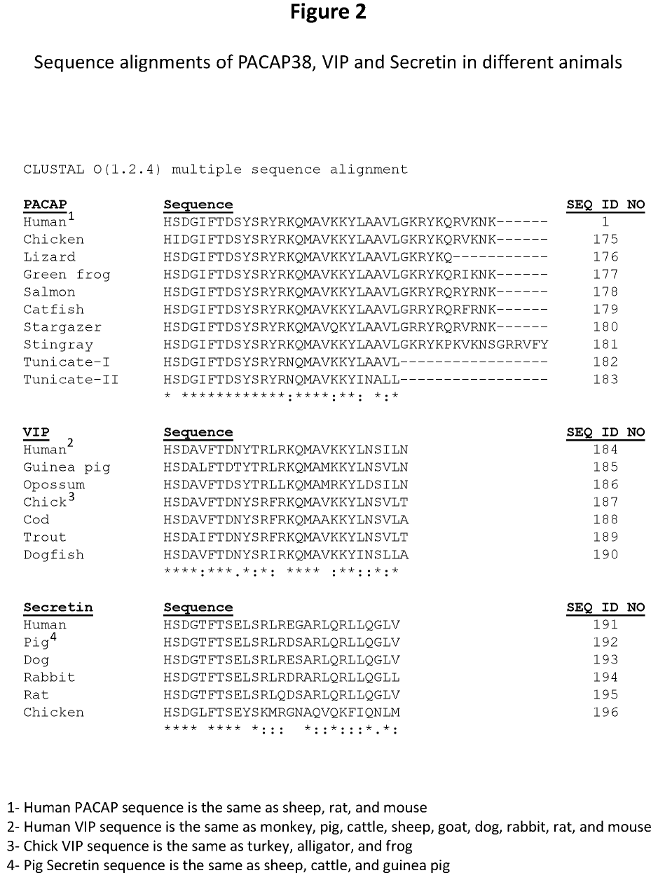

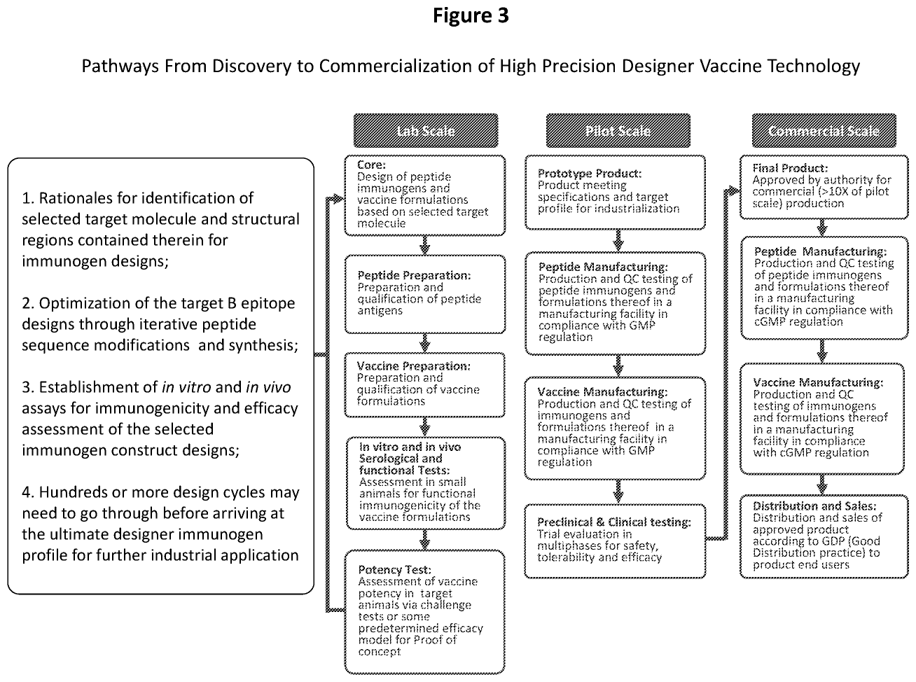

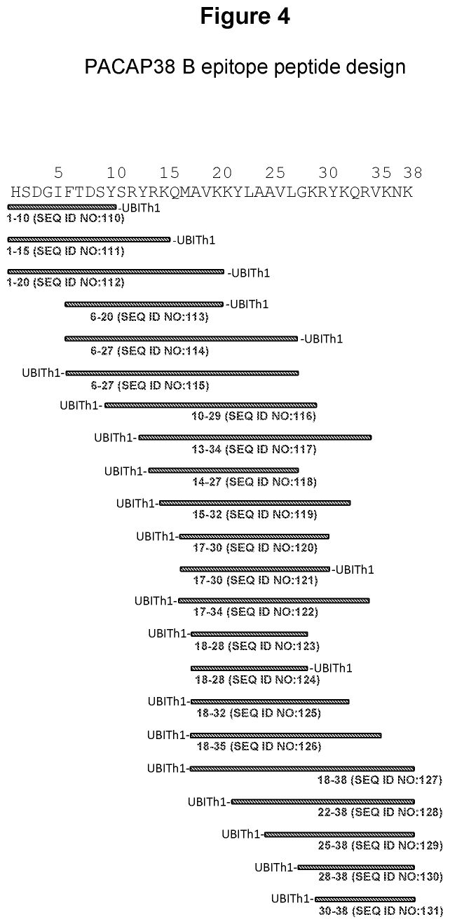

Based on scientific information described in the background section, PACAP is selected as the target molecule for design of the disclosed peptide immunogen constructs. FIG. 1 depicts the pathways from discovery to commercialization (industrialization) of high precision designer synthetic peptide-based vaccine formulations. Detailed evaluation and analyses of each of the steps has led to a myriad of experiments in the past which would ultimately result in commercialization of a safe and efficacious pharmaceutical formulation containing PACAP peptide immunogen constructs.

a. Design History

Each peptide immunogen construct or immunotherapeutic product requires its own design focus and approach based on its specific disease mechanism and the target protein(s) required for intervention. For treatment of pain including headache and migraine, PACAP was selected as the target molecule for intervention. The pathway from discovery to commercialization, as shown in FIG. 1, typically requires one or more decades to accomplish. Identification of the PACAP B cell epitope peptides correlating to the functional site(s) for intervention are key to the immunogen construct design. Consecutive pilot immunogenicity studies in guinea pigs incorporating various T helper support (carrier proteins or suitable T helper peptides) in various formulations were conducted and subsequently evaluated for the functional properties of the elicited purified antibodies or the vaccine formulations employing specific PACAP peptide immunogen constructs in specific in vitro functional assays or proof of concept in vivo studies in selected animal models. Upon extensive serological validation, candidate PACAP B cell epitope peptide immunogen constructs can be further tested in non-human primates to further validate the immunogenicity and direction of the PACAP peptide immunogen design. Selected PACAP peptide immunogen constructs can be prepared in varying mixtures to evaluate the subtle differences in functional properties related to the respective interactions among peptide constructs when used in combinations. Upon additional evaluation, the final peptide constructs, peptide compositions, and pharmaceutical formulations thereof, along with the respective physical parameters of the formulations can be established, leading to the final product development process.

The amino acid sequences of the PACAP peptide immunogen constructs were selected based on a number of design rationales. Several of these rationales include employing a PACAP B cell epitope peptide sequence that:

(i) is devoid of an autologous T helper epitope within PACAP to prevent autologous T cell activation;

(ii) is non-immunogenic on its own, since it is a self-molecule;

(iii) can be rendered immunogenic by a protein carrier or a potent T helper epitope(s) upon administration to a host:

(iv) elicits high titer antibodies directed against the PACAP peptide sequence (B cell epitope) and not against the protein carrier or potent T helper epitope(s);

(v) elicits high titer antibodies that would suppress the induction of intracellular cAMP rise due to PACAP and PACAP receptor interaction and cellular activation; and

(vi) such vaccine formulations, when administered in an animal model, e.g. BALB/C mice, would suppress the dorsal blood flow induced by Capsaicin, as a proof of concept validation for the treatment of pain, including headache and migraine.

b. Design and Validation of PACAP Peptide Immunogen Constructs for Pharmaceutical Compositions with Potential to Treat Patients Predisposed to, or Suffering from, Pain, Including Headache and Migraine

In order to generate the most potent peptide immunogen constructs for incorporation into the pharmaceutical compositions, a repertoire of human PACAP B cell epitope peptides (e.g. SEQ ID NOs: 2-20) and promiscuous T helper epitopes derived from various pathogens or artificially T helper epitopes (e.g. SEQ ID NOs: 70-109 and 160-171) were further designed and made into for example representative PACAP peptide immunogen constructs (e.g., SEQ ID NOs: 110-159) for immunogenicity studies initially in guinea pigs.

i) Selection of PACAP B Cell Epitope Peptide Sequences from the Receptor Binding or Receptor Activation Region for Design

The PAC1 binding region, located at the central/C-terminus of PACAP, and the receptor activation region, located in the N-terminus C2-C7 loop/central region of PACAP, were selected for PACAP B cell epitope design. These B cell epitope peptides were then made into peptide immunogen constructs to elicit immune sera in guinea pigs initially for immunogenicity by ELISA on PACAP B cell epitope peptide coated plates and subsequently for in vitro functional assay assessment.

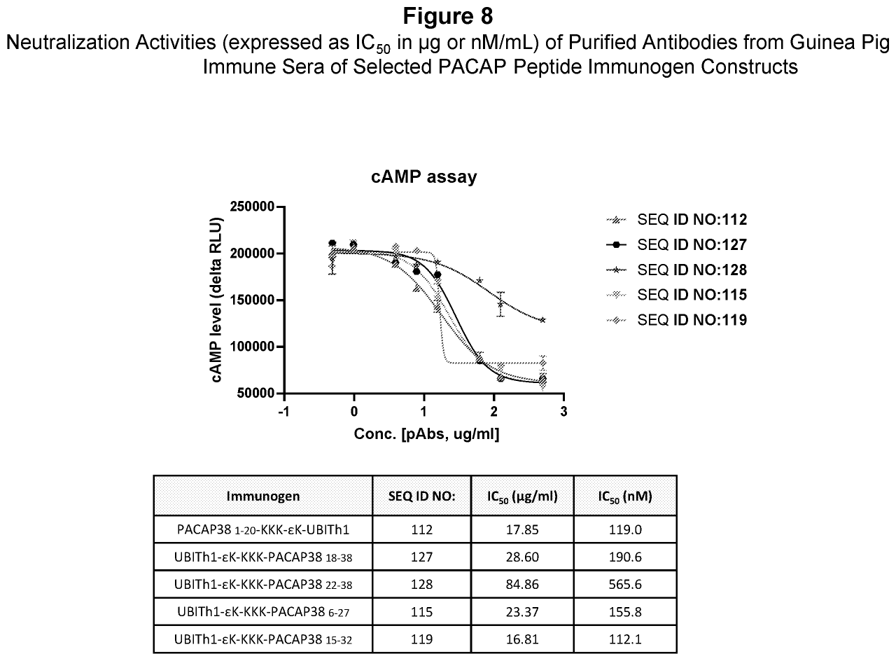

Upon binding of PACAP to PAC1, PAC1 transmits the activation signals intracellularly leading to intracellular rise of cAMP level amongst other cellular events. The ability of purified antibodies from guinea pig immune sera, directed against specific PACAP peptide immunogen constructs, to neutralize the functional properties of PACAP was assessed for IC₅₀ to inhibit 50% of cAMP rise when compared to the control in the absence of antibodies, as shown in FIG. 8 and the tables displayed within FIG. 8.

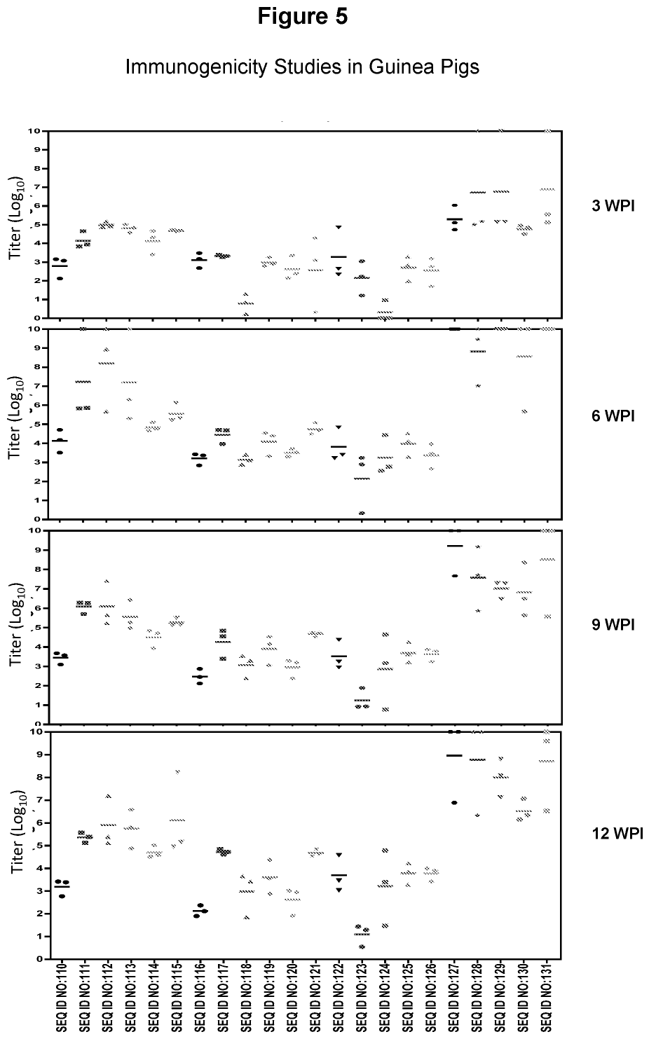

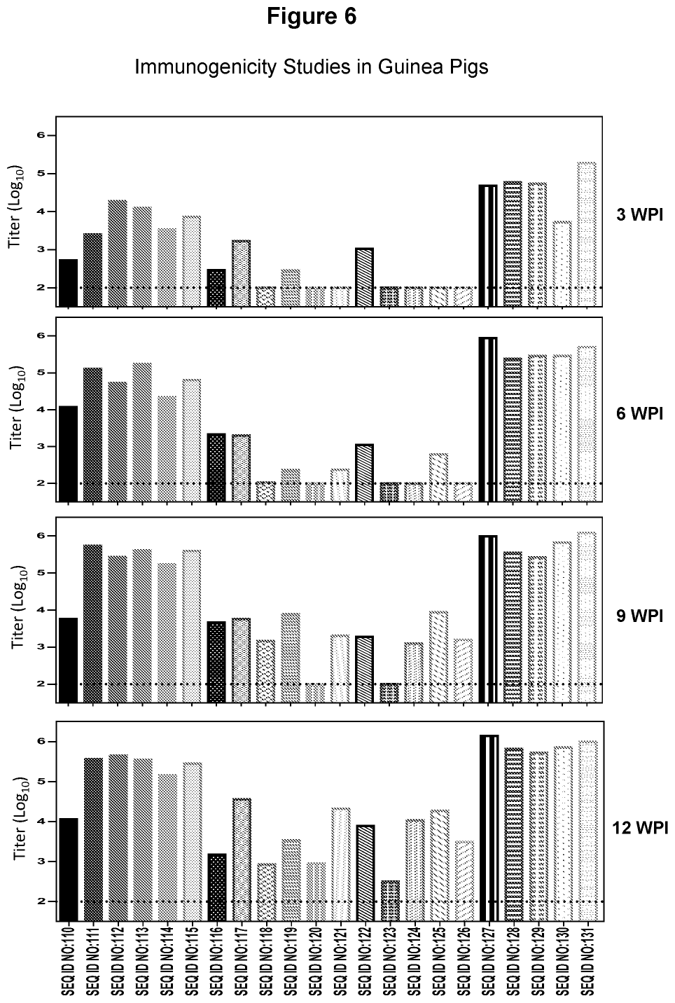

These PACAP peptide immunogen constructs were formulated initially with ISA 51 and CpG for prime immunization in guinea pigs at 400 μg/1 mL and boosts (3, 6, and 9 wpi) at 100 μg/0.25 mL for immunogenicity studies. To test the immunogenicity in guinea pigs, ELISA assay was used with guinea pig immune sera from various (wpi) bleeds, diluted at a 10-fold serial dilution from 1:100 to 1:10,000. ELISA plates were coated with corresponding PACAP B cell epitope peptide and full-length PACAP38 peptide at 0.5 μg peptide per well. The titer of a tested serum, expressed as Log₁₀, was calculated by linear regression analysis of the A450 nm with the cutoff A₄₅₀ set at 0.5, as shown in FIG. 5, with detailed titers for representative B cell epitope derived PACAP peptide immunogen constructs, as shown in Tables 4, 5, and 6. Although the designed short PACAP B cell epitope peptides frequently are non-immunogenic on their own due to their lack of endogenous Th epitopes, addition of foreign Th epitopes enhanced the immunogenicity of the specific PACAP peptide immunogen constructs. Detailed analyses of the reactivity/specificity pattern of various constructs are shown in Tables 4, 5, and 6 along with FIG. 5. The immunogenicity imparted by certain residues within the PACAP molecule can be assessed that would facilitate further design of the optimal peptide immunogen constructs.

ii) Devoid of an Autologous T Helper Epitope within Selected PACAP B Cell Epitope to Prevent Autologous T Cell Activation

Representative PACAP B cell epitopes that were not linked to heterologous Th epitope peptides were tested for their ability to generate antibodies on their own. Experiments were conducted and found that the B cell epitope peptides did not elicit any antibodies to PACAP and, thus, are devoid of the undesirable endogenous Th epitopes within the selected PACAP B cell epitopes (data not shown).

iii) Focused Antibody Response Elicited by PACAP Peptide Immunogen Constructs is Targeted at the PACAP B Cell Epitope Only

It is well known that all carrier proteins (e.g. Keyhole Limpet Hemocyanin (KLH), Diphtheria toxoid (DT) and Tetanus Toxoid (TT) proteins) used to potentiate an immune response directed against the targeted B cell epitope peptide, by chemical conjugation of such B cell epitope peptide to the respective carrier protein, will elicit more than 90% of the antibodies directed against the potentiating carrier protein with less than 10% of the antibodies directed against the targeted B cell epitope in immunized hosts. It is therefore of interest to assess the specificity of the PACAP peptide immunogen constructs of the present invention.

Representative PACAP peptide immunogens (SEQ ID NOs: 111 and 114), containing the B cell epitopes of SEQ ID NOs: 3 and 5, were tested to determine if the antibodies elicited by the peptide immunogen constructs were directed against the B cell epitope portion or the Th epitope portion of the peptide immunogen constructs. As shown in Table 7, the antibodies elicited by these peptide immunogen constructs were specifically directed to the PACAP B cell epitope, and not the heterologous Th epitope portion, of the peptide immunogen constructs.

iv) Fine Epitope Mapping with Immune Sera Directed Against Selected PACAP Peptide Immunogen Constructs

In a fine epitope mapping study to localize the antibody binding site(s) to specific residues within the target B cell epitope region, 46 overlapping 10-mer peptides (SEQ ID NOs: 21-6) were designed that cover from amino acid −10 to amino acid 45 sequence covering the full-length region of PACAP, along with the precursor sequences before and after the processed PACAP molecule. These 10-mer peptides can be individually coated onto 96-well microtiter plate wells as solid-phase immune-absorbents. The pooled guinea pig antisera can be added at a 1:100 dilution in specimen diluent buffer to the plate wells coated with 10-mer peptide at 2.0 μg/mL followed by incubation for one hour at 37° C. After washing the plate wells with wash buffer, the horseradish peroxidase-conjugated rProtein A/G can be added and incubated for 30 min. After washing with PBS again, the substrate can be added to the wells for measurement of absorbance at 450 nm by ELISA plate reader, when the samples are analyzed in duplicate. The binding of immune sera that is elicited by the PACAP peptide immunogens to the corresponding PACAP B cell epitope peptide coated wells would represent the maximal antibody binding signal.

In summary, the designed synthetic PACAP peptide immunogen constructs induce robust immune responses in guinea pigs generating polyclonal antibodies targeted at distinct clusters of 10mer peptides within PACAP, which have close proximity to the PAC1 binding and activation regions, allowing for important medical interventions. Epitope mapping along with functional assay assessment would allow identification of the most optimal peptide immunogen constructs for use in vaccine formulations.

### Example 7

### Assessment of Functional Properties of Antibodies Elicted by the PACAP Peptide Immunnogen Constructs and Formulations Thereof in an Ex-Vivo Mode

After demonstration of the high immunogenicity and cross-reactivities of the antibodies purified from immune sera of guinea pigs immunized with carefully selected candidate PACAP immunogen constructs, as shown in Tables 4, 5, 6, 7, 8, and 9, the following studies were designed to assess whether representative purified IgGs from these immune sera collected at 6 wpi from each animal could suppress intracellular rise of cAMP due to activation by the C2-C7 loop within the PACAP upon binding of PACAP to its receptor, PAC1.

At a molecular level within smooth muscle cells, PACAP could bind to its receptor via its C-terminal region and then activate the receptor using its loop region. The cyclic C2-C7 loop with a disulfide bridge has a basic role in receptor activation that correlates closely with a rise in intracellular cAMP. Various anti-PACAP IgGs were used to characterize their potential anti-PACAP influence in a neutralization assay. The effect was assessed by functional pharmacology using the alterations in intracellular cAMP levels. This in vitro functional assessment is particularly important to assess the anti-PACAP effect of those guinea pig immune sera directed against PACAP peptide immunogen constructs of the current invention with assay procedures detailed in the above Examples.

Suppression of Intracellular cAMP Rise in PACAP Activated Phosphorylation by Anti-PACAP Antibodies

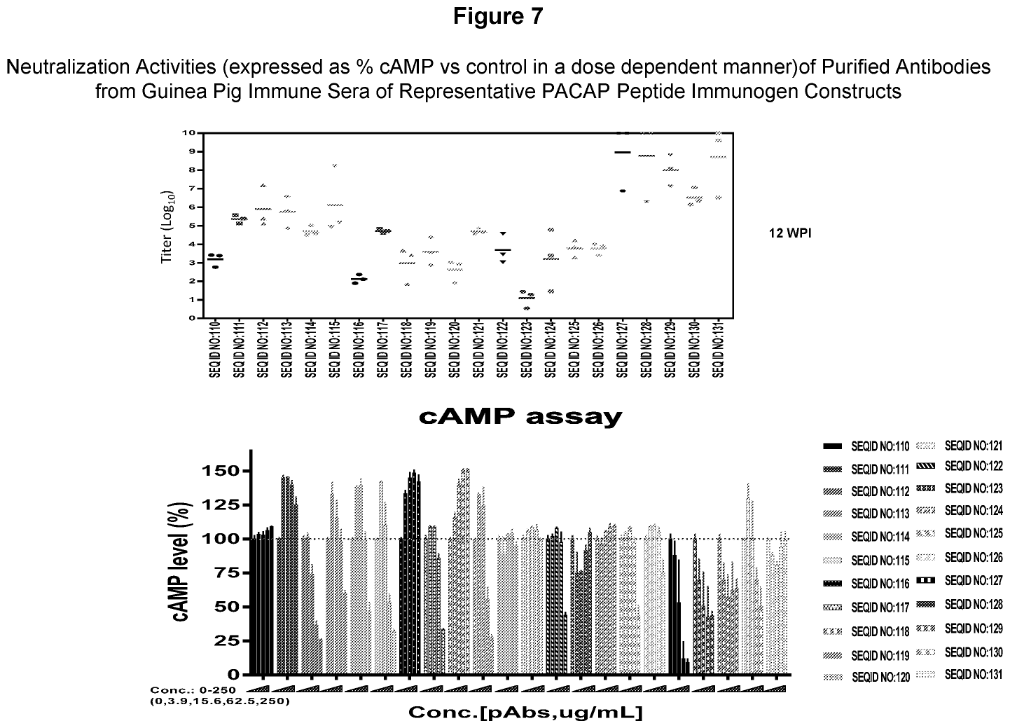

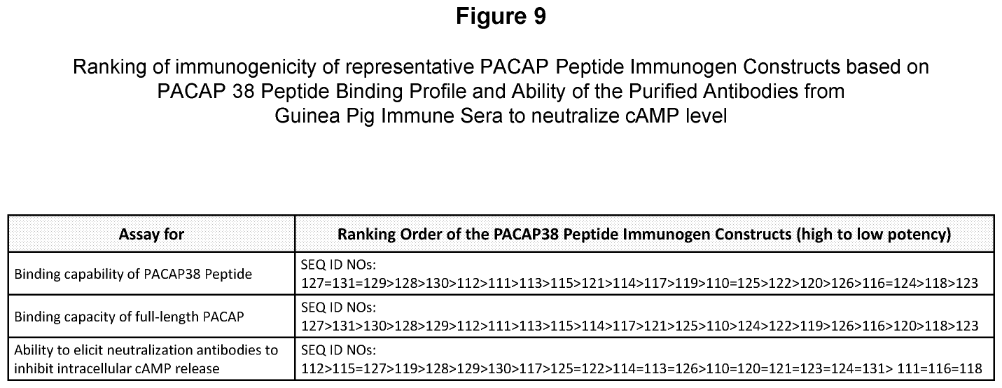

Immune sera from 6 wpi bleeds of each animal were collected with antibodies purified as described in Example 4. Twenty-one PACAP peptide immunogen constructs were tested in guinea pigs for their respective immunogenicity as demonstrated in Example 6. The purified antibodies were grouped into three categories based on their respective target B cell epitope peptides employed in the peptide immunogen constructs. They are those directed at B cell epitope peptides from the N-terminal, Central, and the C-terminal regions, respectively. Data were recorded in percentage of cAMP detected within the PACAP treated L6 cells. Zero percent (0%) represents L6 cell alone and 100% represents PACAP treated L6 cells. As shown in FIGS. 7 and 8, along with the accompanying tables, there are constructs incorporating PACAP B cell epitope peptides from N-terminal, Central, or C-terminal region that showed IC₅₀ (μg/mL) for cAMP level from 0.60 to >20. For practical purpose, IC₅₀ values at less than 10 μg/mL were considered significant in antibody mediated suppression of cAMP production. Representative constructs that showed effective functional immunogenicity are ranked (with IC₅₀ μg/mL from low to high) as SEQ ID NOs: 130>127>150>125>137>123>121>119>124>131>126>133>120>129>135>116˜117˜118˜128˜139, as shown in FIG. 9. These data help to delineate the optimal design for PACAP peptide immunogen constructs with high precision up to a few residues within the PACAP structure.

In summary, PACAP peptide immunogen constructs as shown above in their relative ranking for respective functional properties are of value for use in subsequent PACAP vaccine formulations to demonstrate functional efficacy.

### Example 8

### Capsaicin-Induced Dermal Vasodilation Mouse Model

Three different PACAP38 peptide immunogen constructs were tested for their ability to reduce capsaicin-induced dermal blood flow, as described below.

### Methods

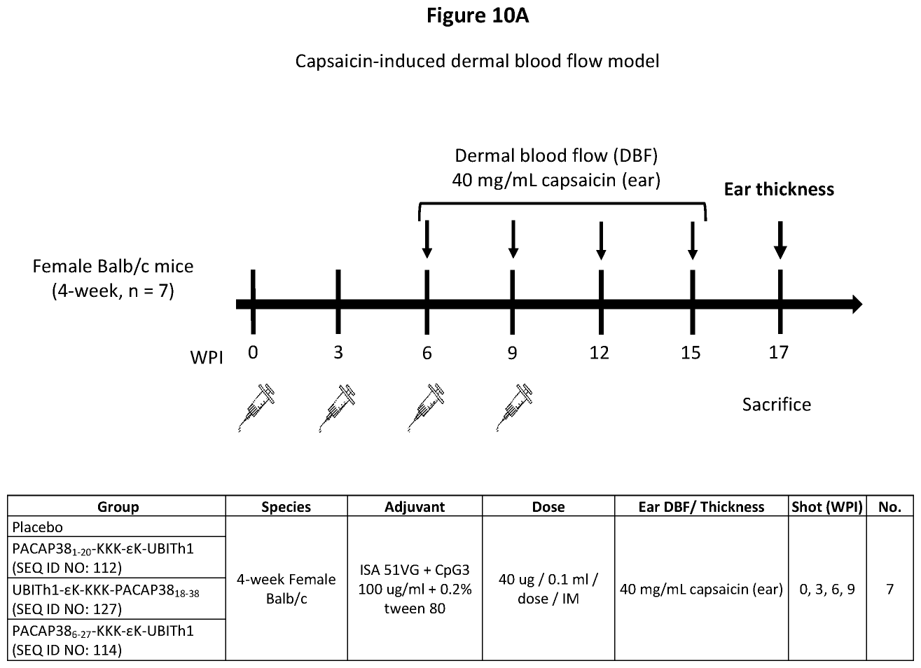

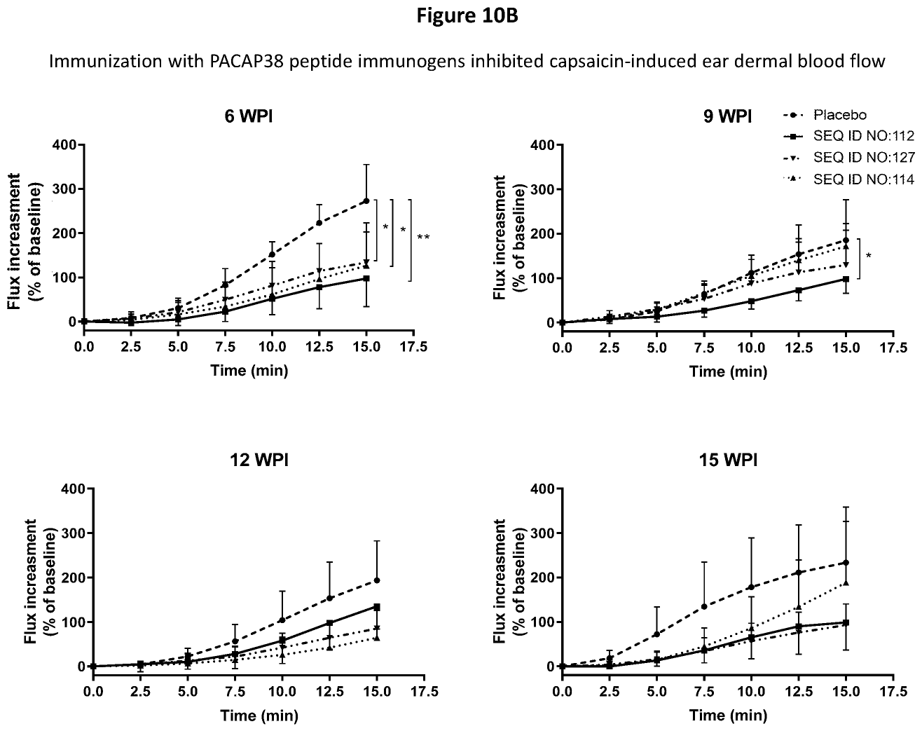

Twenty-eight female BALB/c mice at 4 weeks of age were purchased from BioLASCO Taiwan Co., Ltd. After a 3-day acclimation, animals of each strain were randomly assigned to groups to receive a placebo, or formulations containing the PACAP38 peptide immunogen constructs of SEQ ID NOs: 112, 127, and 114. All procedures on animals were performed in accordance with the regulations and guidelines reviewed and approved by the Institutional Animal Care and Use Committee (IACUC) at UBIA. A schematic summarizing the experiment is shown in FIG. 10A.

BALB/c mice (10-12 weeks, 25-28 g) were housed in cages according to the guidelines at a 12-hour light/dark cycle. Water and standard rodent diet were given ad libitum. A total of twenty-eight mice were assigned randomly into 4 groups (n=7): (1) Placebo, (2) SEQ ID NO: 112, (3) SEQ ID NO: 127, and (4) SEQ ID NO: 114.

The PACAP peptide immunogen constructs of SEQ ID NOs: 112, 127, and 114 were prepared in a water-in-oil emulsion with Seppic MONTANIDE™ ISA 51VG (1:1 in volume), CpG3 at 100 μg/mL, and 0.2% TWEEN80. The mice were immunized with 4 doses of the relevant formulation (placebo or PACAP38 peptide immunogen construct), which were administered at time 0 (prime) and were boosted at 3, 6, and 9 weeks post initial immunization (wpi) by intramuscular route, as shown in FIG. 10A. Mice in groups 2, 3, and 4, receiving formulations containing SEQ ID NOs: 112, 127, and 114, respectively, were dosed with 40 μg of the relevant peptide immunogen construct in a volume of 0.1 ml.

A solution of capsaicin was prepared by dissolving 9.6 mg capsaicin (Sigma, Cat: M2028, Lot: SLCB0726) in 50% EtOh, 33.3% TWEEN20, 16.7% 2dH₂O to make a concentrated 40 mg/mL stock capsaicin solution.

Beginning at 6 wpi, the dermal blood flow (DBF) induced by capsaicin was evaluated for the mice of each group. For the experiments, the mice were anaesthetized (1.5-2 Vol % isoflurane) and placed on a heating pad to maintain their body temperature at a constant 37° C. throughout the experiment. The experiment was conducted for three continuous days. Before the study started, the right ear was flattened for capsaicin application. After the right ear of tested mice was flattened, a baseline scan of the dermal blood flow of ear was obtained using a laser Doppler blood flow monitor (MoorVMS-LDF1, Moor Instruments, Devon UK) equipped with optic probe positioned at a right angle above the skin of the ear. After a baseline scan was obtained, 5 μL containing 0.2 mg of capsaicin (from the 40 mg/mL capsaicin solution) was topically applied to ear skin surface and the blood flow and temperature were monitored. The time course of blood flow response to capsaicin was measured using a laser Doppler monitor for 15 minutes continuously. The data were analyzed using moorVMS-PC-Software.

### Results

The results from this study are provided in FIG. 10B. The data were evaluated by comparing each test group to Placebo group using unpaired two-tailed Student's t-test. P value=0.001 to 0.01, “**”; P value=0.01 to 0.05, “*”; and data are presented as mean with standard error of the mean (±SEM).

The results show that the mice in Group 1 that were given the placebo had the largest flux increase of dermal blood flow (DBF) compared to the mice in Groups 2-4 that received a formulation containing a PACAP38 peptide immunogen construct throughout the course of the study. Thus, the mice in Groups 2-4 consistently had a lower flux increase compared to the mice in Group 1, demonstrating that all of the PACAP38 peptide immunogen constructs tested in this study are capable of reducing capsaicin-induced DBF.

The results at both 6 and 9 wpi show the following ranking from the most reduction in capsaicin-induced DBF to least: SEQ ID NO: 112>127>114>placebo.

The results at 12 wpi show the following ranking from the most reduction in capsaicin-induced DBF to least: SEQ ID NO: 114>127>112>placebo.

The results at 15 wpi show the following ranking from the most reduction in capsaicin-induced DBF to least: SEQ ID NO: 112 127>114>placebo.

Taken together, the results of this study demonstrate that the PACAP38 peptide immunogen constructs tested in this study are capable of reducing capsaicin-induced DBF. In addition, the results for formulations containing SEQ ID NOs: 112, 127, and 114 are consistent with their ability to elicit neutralizing antibodies to inhibit intracellular cAMP release, as shown in FIG. 9.

### Example 9

### Fine Epitope Mapping with Immune Sera Directed Against PACAP38 Peptide Immunogen Constructs

A fine epitope mapping study was performed to localize the antibody binding site(s) to specific residues within the PACAP38 polypeptide.

### Methods

Forty-six (46) overlapping 10-mer peptides (SEQ ID NOs: 21-66) were synthesized that cover the PACAP38 polypeptide from amino acid −10 to amino acid 45, covering the full-length region of PACAP38 along with the precursor sequences before and after the processed PACAP38 polypeptide.

The 10-mer peptides were dissolved in dimethylformamide to a 1 mg/ml stock and kept at −20° C. until used. Peptides were coated overnight at room temperature with 1004 of peptides (2 μg/mL) per well. Guinea pig antisera at 12 wpi for multiple peptide immunogen constructs (SEQ ID NOs: 112, 115, 117, 119, 122, 125, 127, and 128) were used at a dilution of 1/100 but positive control was diluted further (to 1/10,000). Each sample (100 μl/well) was incubated for 2 hours at 37° C. The plates were washed six times with 2504 Wash Buffer (1×). Bound antibodies were detected with standardized preparation HRP-rProtein A/G (1:100) at 37° C. for 1 hour, followed by six washes with Wash Buffer. Finally, 100 μL/well of TMB Substrate Working Solution was added into each well and incubated at 37° C. for 15 minutes in the dark, and the reaction was stopped by adding 100 μL/well of Stop Solution. The absorbance at 450 nm was measured by an ELISA plate reader (Molecular Device, Model: VersaMAx). The results are displayed as 450 nm above 1 as the cut off for positivity. The binding of antibodies from immune sera obtained from animals immunized with PACAP38 peptide immunogen constructs was evaluated against the corresponding PACAP38 B cell epitope peptide coated wells to determine the maximal antibody binding signal.

### Results

The results from this fine epitope mapping experiment are shown in Table 8. A summary of the results is as follows:
a. Peptide immunogen constructs derived from, or containing, the N-terminal region of PACAP38 (SEQ ID NOs: 112 and 115) elicited antibodies with high reactivities against the full-length PACAP38 peptide (SEQ ID NO: 1). These constructs elicited strong antibodies directed toward 10-mer peptides from the N-terminal region (aa3-18) of PACAP38 and were not found to be reactive against other B cell epitope regions of PACAP38.
b. Peptide immunogen constructs derived from, or containing, the Central region of PACAP38 (SEQ ID NOs: 117, 119, 122, and 125) had weak to moderate reactivities against the full-length PACAP38 polypeptide (SEQ ID NO: 1). The peptide immunogen construct of SEQ ID NO: 117 had a moderate reactivity to the full-length PACAP38 polypeptide, while the peptide immunogens of SEQ ID NOs: 119, 122, and 125 only had weak reactivities to the full-length polypeptide. However, the peptide immunogen constructs derived from, or containing, the Central region of PACAP38 (SEQ ID NOs: 117, 119, 122, and 125) were reactive towards the Central region of PACAP38.
c. Peptide immunogen constructs derived from, or containing, B cell epitope peptides covering the C-terminal region of PACAP38 (SEQ ID NOs: 127 and 128) had relatively strong reactivities against the full-length PACAP38 polypeptide (SEQ ID NO: 1) in addition to the C-terminal peptides (aa23-38) of PACAP38.

In summary, the designed synthetic PACAP38 peptide immunogen constructs with B cell epitopes derived from, or containing, the N-terminal or C-terminal regions of PACAP38 induced robust immune responses in guinea pigs that generated polyclonal antibodies targeted against distinct clusters of 10-mer peptides within PACAP38. The epitope mapping study, along with additional functional assay assessment, can provide for the identification of the most optimal peptide immunogen constructs formulations.

TABLE 1
Amino Acid Sequences of PACAP38 and Fragments
Thereof Employed in Serological Assays

| Amino Acid | SEQ | |
|---|---|---|
| positions | ID NO: | Sequence |
| PACAP38 | 1 | HSDGI FTDSY SRYRK QMAVK |
| | | KYLAA VLGKR YKQRV KNK |
| PACAP38 1-10 | 2 | HSDGI FTDSY |
| PACAP38 1-15 | 3 | HSDGI FTDSY SRYRK |
| PACAP38 1-20 | 4 | HSDGI FTDSY SRYRK QMAVK |
| PACAP38 6-20 | 5 | FTDSY SRYRK QMAVK |
| PACAP38 6-27 | 6 | FTDSY SRYRK QMAVK KYLAA VL |
| PACAP38 10-29 | 7 | YSRYR KQMAV KKYLA AVLGK |
| PACAP38 13-34 | 8 | YRKQM AVKKY LAAVL GKRYK QR |
| PACAP38 14-27 | 9 | RKQMA VKKYL AAVL |
| PACAP38 15-32 | 10 | KQMAV KKYLA AVLGK RYK |
| PACAP38 17-30 | 11 | MAVKK YLAAV LGKR |
| PACAP38 17-34 | 12 | MAVKK YLAAV LGKRY KQR |
| PACAP38 18-28 | 13 | AVKKY LAAVL G |
| PACAP38 18-32 | 14 | AVKKY LAAVL GKRYK |
| PACAP38 18-35 | 15 | AVKKY LAAVL GKRYK QRV |
| PACAP38 18-38 | 16 | AVKKY LAAVL GKRYK QRVKN K |
| PACAP38 22-38 | 17 | YLAAV LGKRY KQRVK NK |
| PACAP38 25-38 | 18 | AVLGK RYKQR VKNK |
| PACAP38 28-38 | 19 | GKRYK QRVKN K |
| PACAP38 30-38 | 20 | RYKQR VKNK |
| PACAP38 −10-−1 | 21 | GDDAE PLSKR |
| PACAP38 −9-1 | 22 | DDAEP LSKRH |
| PACAP38 −8-2 | 23 | DSEPL SKRHS |
| PACAP38 −7-3 | 24 | SEPLS KRHSD |
| PACAP38 −6-4 | 25 | EPLSK RHSDG |
| PACAP38 −5-5 | 26 | PLSKR HSDGI |
| PACAP38 −4-6 | 27 | LSKRH SDGIF |
| PACAP38 −3-7 | 28 | SKRHS DGIFT |
| PACAP38 −2-8 | 29 | KRHSD GIFTD |
| PACAP38 −1-9 | 30 | RHSDG IFTDS |
| PACAP38 1-10 | 31 | HSDGI FTDSY |
| PACAP38 2-11 | 32 | SDGIF TDSYS |
| PACAP38 3-12 | 33 | DGIFT DSYSR |
| PACAP38 4-13 | 34 | GIFTD SYSRY |
| PACAP38 5-14 | 35 | IFTDS YSRYR |
| PACAP38 6-15 | 36 | FTDSY SRYRK |
| PACAP38 7-16 | 37 | TDSYS RYRKQ |
| PACAP38 8-17 | 38 | DSYSR YRKQM |
| PACAP38 9-18 | 39 | SYSRY RKQMA |
| PACAP38 10-19 | 40 | YSRYR KQMAV |
| PACAP38 11-20 | 41 | SRYRK QMAVK |
| PACAP38 12-21 | 42 | RYRKQ MAVKK |
| PACAP38 13-22 | 43 | YRKQM AVKKY |
| PACAP38 14-23 | 44 | RKQMA VKKYL |
| PACAP38 15-24 | 45 | KQMAV KKYLA |
| PACAP38 16-25 | 46 | QMAVK KYLAA |
| PACAP38 17-26 | 47 | MAVKK YLAAV |
| PACAP38 18-27 | 48 | AVKKY LAAVL |
| PACAP38 19-28 | 49 | VKKYL AAVLG |
| PACAP38 20-29 | 50 | KKYLA AVLGK |
| PACAP38 21-30 | 51 | KYLAA VLGKR |
| PACAP38 22-31 | 52 | YLAAV LGKRY |
| PACAP38 23-32 | 53 | LAAVL GKRYK |
| PACAP38 24-33 | 54 | AAVLG KRYKQ |
| PACAP38 25-34 | 55 | AVLGK RYKQR |
| PACAP38 26-35 | 56 | VLGKR YKQRV |
| PACAP38 27-36 | 57 | LGKRY KQRVK |
| PACAP38 28-37 | 58 | GKRYK QRVKN |
| PACAP38 29-38 | 59 | KRYKQ RVKNK |
| PACAP38 30-39 | 60 | RYKQR VKNKG |
| PACAP38 31-40 | 61 | YKQRV KNKGR |
| PACAP38 32-41 | 62 | KQRVK NKGRR |
| PACAP38 33-42 | 63 | QRVKN KGRRI |
| PACAP38 34-43 | 64 | RVKNK GRRIA |
| PACAP38 35-44 | 65 | VKNKG RRIAY |
| PACAP38 36-45 | 66 | KNKGR RIAYL |
| PACAP38 −10-45 | 197 | GDDAE PLSKR HSDGI FTDSY |
| | | SRYRK QMAVK KYLAA |
| | | VLGKR YKQRV KNKGR RIAYL |
| Flexible | 67 | PPXPXP |
| Hinge | | |
| Spacer | | |
| Spacer1 | 68 | ϵK-KKK |
| Spacer2 | 69 | KKK-ϵK |

TABLE 2
Amino Acid Sequences of Pathogen Protein
Derived Th Epitopes Including Idealized
Artificial Th Epitopes for Employment
in the Design of PACAP38 Peptide Immunogen
Constructs

| | SEQ ID | |
|---|---|---|
| Description | NO: | Sequence |
| Clostridium | 70 | KKQYIKANSKFIGITEL |
| tetani1 Th | | |
| (UBITh®6) | | |
| MVF1 Th | 71 | LSEIKGVIVHRLEGV |
| (UBITh®5) | | |
| Bordetella | 72 | GAYARCPNGTRALTVA |
| pertussis Th | | ELRGNAEL |
| (UBITh®7) | | |
| Clostridium | 73 | WVRDIIDDFTNESSQKT |
| tetani2 Th | | |
| Diphtheria Th | 74 | DSETADNLEKTVAALSI |
| | | LPGHGC |
| Plasmodium | 75 | DHEKKHAKMEKASS |
| falciparum Th | | VFNVVNS |
| Schistosoma | 76 | KWFKTNAPNGVDEKHRH |
| mansoni Th | | |
| Cholera | 77 | ALNIWDRFDVFCTLGA |
| Toxin Th | | TTGYLKGNS |
| MvF2 Th | 78 | ISEIKGVIVHKIEGI |
| KKKMVF3 Th | 79 | KKKISISEIKGVIVHKIEGILF |
| | 80 | T RT TR T |
| | 81 | KKKISIXEIXXVIVXXIEXILE |
| HBsAg 1 Th | 82 | KKKLFLLTKLLTLPQSLD |
| | 83 | RRRIKII RII I L IR |
| | 84 | VRVV VV V I V |
| | 85 | F FF FF F V F |
| | 86 | F |
| | 87 | XXXXXXXTXXXTXPXSXX |
| MVF4 Th | 88 | ISISEIKGVIVHKIETILF |
| (UBITh®3) | 89 | T RT TR |
| | 90 | ISIXEIXXVIVXXIETILF |
| HBsAg2 Th | 91 | KKKIITITRIITIPQSLD |
| | 92 | FFLL L ITTI |
| | 93 | KKKXXXXTRIXTIXXXXD |
| MVF5 Th | 94 | ISITEIKGVIVHRIETILF |
| (UBITh®1) | | |
| HBsAg3 Th | 95 | KKKIITITRIITIITTID |
| (UBITh®2) | | |
| Influenza | 96 | FVFTLTVPSER |
| MP1_1 Th | | |
| Influenza | 97 | SGPLKAEIAQRLEDV |
| MP1_2 Th | | |
| Influenza | 98 | DRLRRDQKS |
| NSP1 Th | | |
| EBV BHRF1 Th | 99 | AGLTLSLLVICSYLFISRG |
| Clostridium | 100 | QYIKANSKFIGITEL |
| tetani TT1 Th | | |
| EBV EBNA-1 Th | 101 | PGPLRESIVCYFMVFLQTHI |
| Clostridium | 102 | FNNFTVSFWLRVPKVSASHLE |
| tetani TT2 Th | | |
| Clostridium | 103 | KFIIKRYTPNNEIDSF |
| tetani TT3 Th | | |
| Clostridium | 104 | VSIDKFRIFCKALNPK |
| tetani TT4 Th | | |
| EBV CP Th | 105 | VPGLYSPCRAFFNKEELL |
| HCMV IE1 Th | 106 | DKREMWMACIKELH |
| EBV GP340 Th | 107 | TGHGARTSTEPTTDY |
| EBV BPLF1 Th | 108 | KELKRQYEKKLRQ |
| EBV EBNA-2 Th | 109 | TVFYNIPPMPL |
| MvF Th | 160 | DLSDLKGLLLHKLDGL |
| (SSAL1 Th1) | 161 | EI EIR III RIE I |
| | 162 | V V VVV V V |
| | 163 | F F FFF F F |
| | 164 | XXSXXXGXXXHXXXGX |
| MvF3 Th | 165 | ISISEIKGVIVHKIEGILF |
| | 166 | T RT TR T |
| | 167 | ISIXEIXXVIVXXIEXILF |
| KKKMvF5 Th | 168 | KKKISITEIKGVIVHRIETILF |
| (UBITh®1a) | | |
| HBsAg Th | 169 | FFLLTRILTIPQSLD |
| (UBITh®4) | | |
| KKK-HBsAg Th | 170 | KKKFFLLTRILTIPQSLD |
| HBsAg Th | 171 | FFLLTRILTIPQSL |

TABLE 3
Amino Acid Sequences of PAC AP38 Peptide Immuogen Constructs

| | SEQ ID | | | |
|---|---|---|---|---|
| Peptide Description | NO: | Sequence | | |
| PACAP38 1-10-KKK-ϵK-UBITh1 | 110 | | HSDGIFTDSY | -KKK-ϵK-UBITh1 |
| PACAP38 1-15-KKK-ϵK-UBITh1 | 111 | | HSDGIFTDSYSRYRK | -KKK-ϵK-UBITh1 |
| PACAP38 1-20-KKK-ϵK-UBITh1 | 112 | | HSDGIFTDSYSRYRKQMAVK | -KKK-ϵK-UBITh1 |
| PACAP38 6-20-KKK-ϵK-UBITh1 | 113 | | FTDSYSRYRKQMAVK | -KKK-ϵK-UBITh1 |
| PACAP38 6-27-KKK-ϵK-UBITh1 | 114 | | FTDSYSRYRKQMAVKKYLAAVL | -KKK-ϵK-UBITh1 |
| UBITh1-ϵK-KKK-PACAP38 8-27 | 115 | UBITh1-ϵK-KKK- | FTDSYSRYRKQMAVKKYLAAVL | |
| UBITh1-ϵK-KKK-PACAP38 10-29 | 116 | UBITh1-ϵK-KKK- | YSRYRKQMAVKKYLAAVLGK | |
| UBITh1-ϵK-KKK-PACAP38 13-34 | 117 | UBITh1-ϵK-KKK- | YRKQMAVKKYLAAVLGKRYKQR | |
| UBITh1-ϵK-KKK-PACAP38 14-27 | 118 | UBITh1-ϵK-KKK- | RKQMAVKKYLAAVL | |
| UBITh1-ϵK-KKK-PACAP38 15-32 | 119 | UBITh1-ϵK-KKK- | KQMAVKKYLAAVLGKRYK | |
| UBITh1-ϵK-KKK-PACAP38 17-30 | 126 | UBITh1-ϵK-KKK- | MAVKKYLAAVLGKR | |
| PACAP38 17-30-KKK-ϵK-UBITh1 | 121 | | MAVKKYLAAVLG | -KKK-ϵK-UBITh1 |
| UBITh1-ϵK-KKK-PACAP38 17-34 | 122 | UBITh1-ϵK-KKK- | MAVKKYLAAVLGKRYKQR | |
| UBITh1-ϵK-KKK-PACAP38 18-28 | 1Z3 | UBITh1-ϵK-KKK- | AVKKYLAAVLG | |
| PACAP38 18-28-KKK-ϵK-UBITh1 | 124 | | AVKKYLAAVLG | -KKK-ϵK-UBITh1 |
| UBITh1-ϵK-KKK-PACAP38 13-32 | 128 | UBITh1-ϵK-KKK- | AVKKYLAAVLGKRYK | |
| UBITh1-ϵK-KKK-PACAP38 18-35 | 126 | UBITh1-ϵK-KKK- | AVKKYLAAVLGKRYKQRV | |
| UBITh1-ϵK-KKK-PACAP38 18-28 | 12? | UBITh1-ϵK-KKK- | AVKKYLAAVLGKRYKQRVKNK | |
| UBITh1-ϵK-KKK-PACAP38 22-38 | 123 | UBITh1-ϵK-KKK- | YLAAVLGKRYRQRVKNK | |
| UBITh1-ϵK-KKK-PACAP38 26-38 | 129 | UBITh1-ϵK-KKK- | AVLGKRYRQRVKNK | |
| UBITh1-ϵK-KKK-PACAP33 28-38 | 130 | UBITh1-ϵK-KKK- | GKRYKQRVKNR | |
| UBITH1-ϵK-KKK-PACAP38 30-38 | 131 | UBITh1-ϵK-KKK- | RYSGRVKNK | |
| Clostridium tetani Th-ϵK-KKK-PACAP38 18-38 | 132 | KKQYIKANSKFIGITEL-ϵK-KKK- | AVKKYLAAVLGKRYKQRVKNK | |
| MvF1 Th-ϵK-KKK-PACAP38 18-38 | 133 | LSEIKGVIVHRLEGV-ϵK-KKK- | AVKKYLAAVLGKRYKQRVKNK | |
| Bordetella pertussis Th-ϵK-KKK-PACAP38 18-38 | 134 | GAYARCFNGTRALTVAELRGNAEL-ϵK-KKK- | AVKKYLAAVLGKRYKQRVKNK | |
| Clostridium tetani 2 Th-ϵK-KKK-PACAP38 18-38 | 135 | WVRDIIDDFTNESSQKT-ϵK-KKK- | AVKKYLAAVLGKRYKQRVKNK | |
| Diphteria Th-ϵK-KKK-PACAP38 18-38 | 138 | DSETADNLEKTVAALSILPGHGC-ϵK-KKK- | AVKKYLAAVLGKRYKQRVKNK | |
| Pasmodxim tatciparum Th-ϵK-KKK-PACAP38 18-38 | 137 | DHEKKHAKMSKASSVFNVVNS-ϵK-KKK- | AVKKYLAAVLGKRYKQRVKNK | |
| Schistosoma mansoni Th-ϵK-KKK-PACAP38 18-38 | 138 | KWFKTNAPNGVDEKHRH-ϵK-KKK- | AVKKYLAAVLGKRYKQRVKNK | |
| Cholera Toxin Th-ϵK-KKK-PACAP38 18-38 | 139 | ALNIWDRFDVFCTLGATTGYLKGNS-ϵK-KKK- | AVKKYLAAVLGKRYKQRVKNK | |
| MF2 Th-ϵK-KKK-PACAP38 18-38 | 140 | ISEIKGVIVHKIEGILF-ϵK-KKK- | AVKKYLAAVLGKRYKQRVKNK | |
| KKRMYF3 Th-ϵK-KKK-PACAP38 18-38 | 141 | KKKISISELKGVHKIEGILF-ϵK-KKK- | AVKKYLAAVLGKRYKQRVKNK | |
| | | T RT TR T | | |
| HBsAg1 Th-ϵK-KKK-PACAP38 18-38 | 142 | KKKLFLLTKLLTLPQSLD-ϵK-KKK- | AVKKYLAAVLGKRYKQRVKNK | |
| | | RRRIKII RII I L IR | | |
| | | VRVV VV V I V | | |
| | | F FF FF F V F | | |
| | | F | | |
| MF4 Th (UBITh®2)-ϵK-KKK-PACAP38 18-38 | 143 | ISISEIKGVIVHKIETILF-ϵK-KKK- | AVKKYLAAVLGKRYKQRVKNK | |
| | | T RT TR | | |
| HBsAg2 Th-ϵK-KKK-PACAP38 18-38 | 144 | KKKIITITRIITIPQSLD-ϵK-KKK- | AVKKYLAAVLGKRYKQRVKNK | |
| | | FFLL L ITTI | | |
| HBsAg3 Th (UBITh®2)-ϵK-KKK-PACAP38 18-38 | 145 | KKKIITITRIITIITTID-ϵK-KKK- | AVKKYLAAVLGKRYKQRVKNK | |
| Influenza MP1_1 Th-ϵK-KKK-PACAP38 18-38 | 145 | FVFLTVPSER-ϵK-KKK- | AVKKYLAAVLGKRYKQRVKNK | |
| Inficenza MP1_2 Th-ϵK-KKK-PACAP38 18-38 | 147 | SGPLKAEIAQRLEDV-ϵK-KKK- | AVKKYLAAVLGKRYKQRVKNK | |
| Influenza NSP1 Th-ϵK-KKK-PACAP38 18-38 | 148 | DRLRRDQKS-ϵK-KKK- | AVKKYLAAVLGKRYKQRVKNK | |
| EBV BHRF1 Th-ϵK-KKK-PACAP38 18-38 | 149 | AGLTLSLLVICSYFLISRG-ϵK-KKK- | AVKKYLAAVLGKRYKQRVKNK | |
| Clostridium tetani TT1 Th-ϵK-KKK-PACAP38 18-38 | 150 | QYIKANSKFIGITEL-ϵK-KKK- | AVKKYLAAVLGKRYKQRVKNK | |
| EBV EBNA-1 Th-ϵK-KKK-PACAP38 18-38 | 151 | PGPLRESIVCYFMVFLQTHI-ϵK-KKK- | AVKKYLAAVLGKRYKQRVKNK | |
| Clostridium tetani TT2 Th-ϵK-KKK-PACAP38 18-38 | 152 | FNNFTVSFWLRVPEVSASHLE-ϵK-KKK- | AVKKYLAAVLGKRYKQRVKNK | |
| Clostridiam tetani TT3 Th-sK-KKK-PACAP3818-33 | 153 | KFIIKRYTPNNEIDSF-ϵK-KKK- | AVKKYLAAVLGKRYKQRVKNK | |
| Clostridium tetani TT4 Th-ϵK-KKK-PACAP38 18-38 | 154 | VSIDKFRIFCKALNPK-ϵK-KKK- | AVKKYLAAVLGKRYKQRVKNK | |
| EEV CP Th-ϵK-KKK-PACAP38 18-38 | 155 | VPGLYSPCRAFYNKEELL-ϵK-KKK- | AVKKYLAAVLGKRYKQRVKNK | |
| HCMV IE1 Th-ϵK-KKK-PACAP38 18-38 | 150 | DKREMWMACIKELH-ϵK-KKK- | AVKKYLAAVLGKRYKQRVKNK | |
| EEV GP340 Th-KKK-PAOAP33 18-38 | 157 | TGHGARTSTEPTTDY-ϵK-KKK- | AVKKYLAAVLGKRYKQRVKNK | |
| EBV BPLF1 Th-ϵK-KKK-PACAP38 18-38 | 158 | KELKRQYEKKLRQ-ϵK-KKK- | AVKKYLAAVLGKRYKQRVKNK | |
| EBV ESNA-2 Th-ϵK-KKK-PACAP38 18-38 | 159 | TYFYNIPPMPL-ϵK-KKK- | AVKKYLAAVLGKRYKQRVKNK | |

TABLE 4A
Immunogenicity Assessment in Guinea Pigs of PACAP38 Peptide Immunogen Constructs

| | | | | PACAP38₁₋₃₀ | | | | | PACAP38₁₃₋₃₄ |
|---|---|---|---|---|---|---|---|---|---|
| | Peptide | | | (SEQ ID NO: 4) | | | | | (SEQ ID NO: 8) |
| Group | immunogen | SEQ ID | Animal | ELISA Log₁₀ Titer | | | | | ELISA Log₁₀ Titer |
| # | description | NO: | No | 0 w | 3 w | 6 w | 9 w | 12 w | 0 w |
| 1 | PACAP38₁₋₁₀- | 110 | 7826 | 0.065 | 2.867 | 3.970 | 4.512 | 4.246 | 0.097 |
| | KKK-εK-UBITh1 | | 7827 | 0.074 | 3.103 | 4.840 | 4.547 | 4.357 | 0.097 |
| | | | 7828 | 0.066 | 3.253 | 4.697 | 3.669 | 3.319 | 0.118 |
| 2 | PACAP38₁₋₂₀- | 112 | 7829 | 0.071 | 4.868 | 5.097 | 4.962 | 4.905 | 0.099 |
| | KKK-εK-UBITh1 | | 7830 | 0.073 | 4.613 | 5.001 | 4.899 | 4.790 | 0.112 |
| | | | 7831 | 0.064 | 4.624 | 5.083 | 5.042 | 5.058 | 0.106 |
| 3 | UBITh1-εK- | 117 | 7832 | 0.060 | 0.000 | 0.000 | 0.000 | 0.220 | 0.084 |
| | KKK-PACAP3813-34 | | 7833 | 0.060 | 0.000 | 0.000 | 0.000 | 0.000 | 0.091 |
| | | | 7834 | 0.056 | 0.000 | 0.000 | 0.000 | 0.000 | 0.075 |
| 4 | UBITh1-εK- | 122 | 7835 | 0.082 | 0.000 | 0.000 | 0.000 | 1.307 | 0.079 |
| | KKK-PACAP38₁₇₋₃₄ | | 7836 | 0.067 | 0.000 | 0.000 | 0.000 | 1.089 | 0.095 |
| | | | 7837 | 0.078 | 0.000 | 0.000 | 0.000 | 1.659 | 0.104 |
| 5 | UBITh1-εK- | 120 | 7838 | 0.063 | 0.000 | 0.000 | 0.000 | 2.734 | 0.125 |
| | KKK-PACAP38₁₇₋₃₀ | | 7839 | 0.076 | 0.000 | 0.000 | 0.000 | 1.491 | 0.109 |
| | | | 7840 | 0.065 | 0.000 | 0.000 | 0.000 | 2.594 | 0.094 |
| 6 | PACAP38₁₇₋₃₀- | 121 | 7841 | 0.060 | 0.000 | 0.000 | 0.000 | 0.000 | 0.078 |
| | KKK-εK-UBITh1 | | 7842 | 0.067 | 0.000 | 0.000 | 0.000 | 0.000 | 0.078 |
| | | | 7843 | 0.073 | 0.000 | 0.000 | 0.000 | 0.000 | 0.106 |
| 7 | UBITh1-εK- | 123 | 7844 | 0.075 | 0.000 | 0.000 | 0.000 | 0.000 | 0.088 |
| | KKK-PACAP3₁₈₋₂₈ | | 7845 | 0.064 | 0.000 | 0.000 | 0.000 | 0.000 | 0.098 |
| | | | 7846 | 0.060 | 0.000 | 0.000 | 0.000 | 0.000 | 0.106 |
| 8 | PACAP38₁₈₋₂₈- | 124 | 7847 | 0.058 | 0.000 | 0.000 | 0.000 | 0.000 | 0.100 |
| | KKK-εK-UBITh1 | | 7848 | 0.063 | 0.000 | 0.000 | 0.000 | 0.000 | 0.064 |
| | | | 7849 | 0.065 | 0.000 | 0.000 | 0.000 | 0.000 | 0.073 |
| 9 | UBITh1-εK- | 126 | 7850 | 0.060 | 0.000 | 0.000 | 0.000 | 0.000 | 0.072 |
| | KKK-PACAP38₁₈₋₃₅ | | 7851 | 0.080 | 0.000 | 0.000 | 0.000 | 0.000 | 0.068 |
| | | | 7852 | 0.067 | 0.000 | 0.000 | 0.000 | 0.000 | 0.096 |
| 10 | UBITh1-εK- | 125 | 7853 | 0.066 | 0.000 | 0.000 | 0.000 | 0.000 | 0.088 |
| | KKK-PACAP38₁₈₋₃₂ | | 7854 | 0.059 | 0.000 | 0.000 | 0.000 | 0.000 | 0.088 |
| | | | 7855 | 0.061 | 0.000 | 0.000 | 0.000 | 1.145 | 0.077 |
| 11 | UBITh1-εK- | 127 | 7856 | 0.054 | 0.000 | 0.000 | 0.000 | 0.000 | 0.066 |
| | KKK-PACAP38₁₈₋₃₈ | | 7857 | 0.054 | 0.000 | 0.000 | 0.000 | 0.000 | 0.065 |
| | | | 7858 | 0.059 | 0.000 | 0.000 | 0.000 | 0.000 | 0.061 |
| 12 | UBITh1-εK- | 128 | 7859 | 0.065 | 0.000 | 0.000 | 2.228 | 2.591 | 0.067 |
| | KKK-PACAP38₂₂₋₃₈ | | 7860 | 0.060 | 0.000 | 0.000 | 1.318 | 2.146 | 0.075 |
| | | | 7861 | 0.062 | 0.000 | 0.000 | 0.000 | 0.000 | 0.988 |
| 13 | UBITh1-εK- | 129 | 7862 | 0.056 | 0.000 | 0.000 | 0.000 | 0.000 | 0.087 |
| | KKK-PACAP38₂₅₋₃₈ | | 7863 | 0.055 | 0.000 | 2.636 | 2.114 | 0.901 | 0.075 |
| | | | 7864 | 0.053 | 0.000 | 0.000 | 0.000 | 0.000 | 0.063 |

| | PACAP38₁₃₋₃₄ | | | | PACAP38₁₈₋₃₈ | | | | |
|---|---|---|---|---|---|---|---|---|---|
| | (SEQ ID NO: 8) | | | | (SEQ ID NO: 16) | | | | |
| Group | ELISA Log₁₀ Titer | | | | ELISA Log₁₀ Titer | | | | |
| # | 3 w | 6 w | 9 w | 12 w | 0 w | 3 w | 6 w | 9 w | 12 w |
| 1 | 0.000 | 0.000 | 0.000 | 0.000 | 0.140 | 0.000 | 0.000 | 0.000 | 0.000 |
| | 0.000 | 0.000 | 0.000 | 0.000 | 0.114 | 0.000 | 0.000 | 0.000 | 0.000 |
| | 0.000 | 0.000 | 0.000 | 0.000 | 0.145 | 0.000 | 0.000 | 0.000 | 0.000 |
| 2 | 0.000 | 0.000 | 0.000 | 0.000 | 0.106 | 0.000 | 0.000 | 0.000 | 0.000 |
| | 0.000 | 0.000 | 0.000 | 0.000 | 0.104 | 0.000 | 0.000 | 0.000 | 0.000 |
| | 0.000 | 0.000 | 1.384 | 3.526 | 0.101 | 0.000 | 0.000 | 0.000 | 0.000 |
| 3 | 4.608 | 5.147 | 5.252 | 5.783 | 0.096 | 2.568 | 3.650 | 3.798 | 3.872 |
| | 3.759 | 5.250 | 5.209 | 5.240 | 0.085 | 2.970 | 3.220 | 3.153 | 3.517 |
| | 3.776 | 5.167 | 4.993 | 4.884 | 0.092 | 1.741 | 3.188 | 3.282 | 3.724 |
| 4 | 3.887 | 5.074 | 5.164 | 5.327 | 0.107 | 0.000 | 3.023 | 1.192 | 2.012 |
| | 3.724 | 5.109 | 6.574 | 5.282 | 0.098 | 1.957 | 3.220 | 3.270 | 3.634 |
| | 3.744 | 5.167 | 11.590 | 5.931 | 0.120 | 0.000 | 0.362 | 2.585 | 2.814 |
| 5 | 1.496 | 3.295 | 3.106 | 3.114 | 0.108 | 1.751 | 3.124 | 2.093 | 1.692 |
| | 2.906 | 3.104 | 2.974 | 2.865 | 0.090 | 2.711 | 3.134 | 2.920 | 2.587 |
| | 0.000 | 2.758 | 1.770 | 0.787 | 0.082 | 0.416 | 3.552 | 3.045 | 2.783 |
| 6 | 2.489 | 4.409 | 3.677 | 3.417 | 0.079 | 1.981 | 4.891 | 4.562 | 4.348 |
| | 0.000 | 3.218 | 3.285 | 3.617 | 0.094 | 0.642 | 4.582 | 4.364 | 4.856 |
| | 2.687 | 3.678 | 4.047 | 4.293 | 1.102 | 2.092 | 3.527 | 4.646 | 4.801 |
| 7 | 0.000 | 0.000 | 0.000 | 0.000 | 0.123 | 0.000 | 0.158 | 0.000 | 0.000 |
| | 0.000 | 0.000 | 0.000 | 0.000 | 0.109 | 0.000 | 0.000 | 0.000 | 0.000 |
| | 1.198 | 2.453 | 0.523 | 0.000 | 0.098 | 0.000 | 3.028 | 0.000 | 0.000 |
| 8 | 0.000 | 1.204 | 2.726 | 3.088 | 0.089 | 3.226 | 4.982 | 5.108 | 4.920 |
| | 0.000 | 3.282 | 4.001 | 4.547 | 0.090 | 1.592 | 5.085 | 5.040 | 5.002 |
| | 0.000 | 0.875 | 0.000 | 0.000 | 0.089 | 2.470 | 5.033 | 4.795 | 4.499 |
| 9 | 0.803 | 2.688 | 3.106 | 3.335 | 0.091 | 2.315 | 3.212 | 3.336 | 3.411 |
| | 1.758 | 3.294 | 3.832 | 4.679 | 0.096 | 2.347 | 2.929 | 3.135 | 3.208 |
| | 0.000 | 0.000 | 2.681 | 2.834 | 0.122 | 0.000 | 0.000 | 2.820 | 3.073 |
| 10 | 0.000 | 2.563 | 2.242 | 2.593 | 0.126 | 0.000 | 3.461 | 3.548 | 3.436 |
| | 1.836 | 2.048 | 2.595 | 3.025 | 0.113 | 2.968 | 3.075 | 3.046 | 3.099 |
| | 2.167 | 2.857 | 2.953 | 3.248 | 0.124 | 3.021 | 3.468 | 3.471 | 3.595 |
| 11 | 0.006 | 4.917 | 4.734 | 4.792 | 0.078 | 4.956 | >10 | >10 | 7.618 |
| | 0.000 | 3.687 | 4.816 | 4.875 | 0.076 | 4.267 | >10 | >10 | 9.254 |
| | 0.241 | 3.574 | 4.700 | 4.900 | 0.078 | 5.227 | >10 | >10 | >10 |
| 12 | 2.376 | 3.312 | 4.640 | 4.800 | 0.096 | 4.884 | 7.237 | 7.082 | 6.498 |
| | 0.307 | 4.112 | 4.821 | 4.856 | 0.100 | 4.834 | 5.927 | 7.229 | 6.581 |
| | 0.000 | 2.600 | 3.321 | 4.400 | 0.115 | 8.630 | 7.001 | 5.637 | 5.525 |
| 13 | 1.058 | 4.061 | 4.656 | 4.851 | 0.114 | >10 | 9.109 | 6.220 | 5.830 |
| | 0.000 | 2.851 | 3.071 | 3.510 | 0.120 | 5.008 | 8.542 | 6.637 | 6.310 |
| | 0.000 | 3.540 | 4.191 | 4.612 | 0.097 | 5.020 | 6.978 | 5.940 | 5.959 |

TABLE 4B

| | | | | PACAP38₁₋₂₀ | | | | | PACAP38₁₀₋₂₈ | | | | | PACAP38₂₈₋₃₈ | | | | |
|---|---|---|---|---|---|---|---|---|---|---|---|---|---|---|---|---|---|---|
| | Peptide | SEQ | Ani- | (SEQ ID NO: 4) | | | | | (SEQ ID NO: 7) | | | | | (SEQ ID NO: 19) | | | | |
| Group | immunogen | ID | mal | ELISA Log₁₀ Titer | | | | | ELISA Log₁₀ Titer | | | | | ELISA Log₁₀ Titer | | | | |
| # | description | NO: | No | 0 w | 3 w | 6 w | 9 w | 12 w | 0 w | 3 w | 6 w | 9 w | 12 w | 0 w | 3 w | 6 w | 9 w | 12 w |
| 1 | PACAP38₁₋₁₅- | 111 | 7886 | 0.072 | 4.424 | 5.042 | 5.109 | 5.009 | 0.071 | 0.000 | 0.000 | 0.000 | 1.766 | 0.065 | 0.000 | 0.000 | 0.000 | 0.000 |
| | KKK-εK- | | 7887 | 0.075 | 3.635 | 5.083 | 5.026 | 4.889 | 0.075 | 0.000 | 2.348 | 0.000 | 0.000 | 0.066 | 0.000 | 0.000 | 0.000 | 0.000 |
| | UBITh1 | | 7888 | 0.071 | 4.001 | 5.134 | 5.015 | 4.806 | 0.071 | 0.000 | 2.955 | 0.435 | 0.380 | 0.070 | 0.000 | 0.000 | 0.000 | 0.000 |
| 2 | PACAP38₆₋₂₇- | 114 | 7889 | 0.141 | 0.063 | 2.974 | 2.524 | 2.471 | 0.085 | 3.080 | 3.669 | 3.051 | 3.204 | 0.073 | 0.000 | 0.000 | 0.000 | 0.000 |
| | KKK-εK- | | 7890 | 0.156 | 3.179 | 4.611 | 4.537 | 4.610 | 0.083 | 2.643 | 3.044 | 2.890 | 2.891 | 0.064 | 0.000 | 0.000 | 0.389 | 0.000 |
| | UBITh1 | | 7891 | 0.111 | 2.504 | 3.290 | 3.232 | 3.206 | 0.081 | 0.000 | 2.384 | 1.100 | 1.510 | 0.061 | 0.000 | 0.000 | 0.000 | 0.000 |
| 3 | UBITh1-εK- | 115 | 7892 | 0.083 | 3.343 | 4.835 | 4.857 | 4.778 | 0.070 | 2.241 | 4.210 | 4.157 | 3.869 | 0.060 | 0.000 | 0.000 | 0.000 | 0.000 |
| | KKK- | | 7893 | 0.068 | 3.017 | 4.853 | 5.000 | 5.030 | 0.070 | 0.000 | 3.480 | 4.363 | 4.627 | 0.059 | 0.000 | 0.000 | 0.000 | 0.000 |
| | PACAP38₆₋₂₇ | | 7894 | 0.082 | 3.833 | 4.681 | 4.734 | 4.713 | 0.061 | 0.000 | 2.966 | 2.984 | 3.112 | 0.061 | 0.000 | 0.000 | 0.000 | 0.000 |
| 4 | UBITh1-εK- | 116 | 7895 | 0.074 | 0.000 | 0.269 | 0.270 | 0.000 | 0.078 | 2.355 | 3.168 | 3.262 | 3.391 | 0.069 | 0.000 | 0.000 | 0.000 | 0.000 |
| | KKK- | | 7896 | 0.071 | 0.000 | 1.488 | 0.204 | 0.936 | 0.064 | 4.658 | 4.765 | 3.796 | 3.423 | 0.088 | 0.000 | 0.000 | 0.000 | 0.000 |
| | PACAP38₁₀₋₂₉ | | 7897 | 0.082 | 0.000 | 0.000 | 0.684 | 0.000 | 0.080 | 4.951 | 4.649 | 3.708 | 3.291 | 0.060 | 0.000 | 0.000 | 0.000 | 0.000 |
| 5 | UBITh1-εK- | 119 | 7898 | 0.081 | 0.000 | 0.000 | 0.000 | 0.000 | 0.077 | 0.000 | 3.197 | 3.233 | 3.251 | 0.068 | 0.000 | 2.996 | 2.260 | 2.306 |
| | KKK- | | 7899 | 0.068 | 0.000 | 0.000 | 0.000 | 0.000 | 0.079 | 0.000 | 2.997 | 3.038 | 2.953 | 0.067 | 2.586 | 3.602 | 3.191 | 3.093 |
| | PACAP38₁₅₋₃₂ | | 7900 | 0.066 | 0.000 | 0.000 | 0.000 | 0.000 | 0.062 | 1.917 | 2.733 | 1.701 | 0.993 | 0.065 | 0.000 | 0.000 | 0.000 | 0.000 |
| 6 | UBITh1-εK- | 131 | 7901 | 0.057 | 0.000 | 0.000 | 0.199 | 1.603 | 0.069 | 0.000 | 0.000 | 0.000 | 0.000 | 0.059 | 5.231 | >10 | 8.113 | 7.845 |
| | KKK- | | 7902 | 0.056 | 0.000 | 0.000 | 0.000 | 0.000 | 0.064 | 0.000 | 0.000 | 0.000 | 0.000 | 0.069 | 4.955 | 8.014 | 5.471 | 6.709 |
| | PACAP38₃₀₋₃₈ | | 7903 | 0.057 | 0.000 | 0.000 | 0.000 | 0.000 | 0.065 | 0.000 | 0.000 | 0.000 | 0.000 | 0.070 | 7.321 | >10 | 8.697 | 8.391 |
| 7 | PACAP38₆₋₂₀- | 113 | 7904 | 0.055 | 3.155 | 4.942 | 4.904 | 4.994 | 0.067 | 1.523 | 3.325 | 2.669 | 2.844 | 0.070 | 0.000 | 2.980 | 0.044 | 0.000 |
| | KKK-εK- | | 7905 | 0.071 | 4.463 | 5.066 | 5.007 | 4.891 | 0.097 | 0.000 | 3.309 | 2.799 | 2.837 | 0.059 | 0.000 | 0.000 | 0.000 | 0.000 |
| | UBITh1 | | 7906 | 0.071 | 4.789 | 4.966 | 4.838 | 4.680 | 0.091 | 0.000 | 2.770 | 2.752 | 2.423 | 0.065 | 0.000 | 0.000 | 0.000 | 0.000 |
| 8 | UBITh1-εK- | 118 | 7907 | 0.075 | 0.369 | 0.000 | 0.000 | 0.000 | 0.098 | 1.421 | 2.508 | 2.574 | 2.909 | 0.072 | 0.000 | 0.000 | 0.000 | 0.000 |
| | KKK- | | 7908 | 0.062 | 0.000 | 0.000 | 0.000 | 0.000 | 0.077 | 0.000 | 2.318 | 1.301 | 0.973 | 0.065 | 0.000 | 0.000 | 0.000 | 0.000 |
| | PACAP38₁₄₋₂₇ | | 7909 | 0.065 | 0.000 | 0.000 | 0.000 | 0.000 | 0.084 | 0.000 | 2.191 | 3.058 | 3.131 | 0.074 | 0.000 | 3.145 | 1.204 | 1.511 |
| 9 | UBITh1-εK- | 130 | 7910 | 0.063 | 0.000 | 0.000 | 0.000 | 0.000 | 0.073 | 0.000 | 0.000 | 0.000 | 0.000 | 0.069 | 4.557 | >10 | 7.515 | 7.241 |
| | KKK- | | 7911 | 0.061 | 0.000 | 0.000 | 0.000 | 0.000 | 0.085 | 0.000 | 0.000 | 0.000 | 0.000 | 0.074 | 3.663 | 5.266 | 5.282 | 5.542 |
| | PACAP38₂₈₋₃₈ | | 7912 | 0.067 | 0.000 | 0.000 | 0.000 | 0.000 | 0.087 | 0.000 | 0.000 | 0.000 | 0.000 | 0.086 | 4.759 | >10 | 5.528 | 5.554 |

TABLE 5
Immunogenicity Assessment in Guinea Pigs
of PACAP38 Peptide Immunogen Constructs

| | | | PACAP38 (1-38) (SEQ ID NO: 1) | | | | |
|---|---|---|---|---|---|---|---|
| | SEQ ID | Animal | ELISA Log10 Titer | | | | |
| Immunogen | NO: | No | 0 w | 3 W | 6 W | 9 W | 12 W |
| PACAP38₁₋₁₀- | 110 | 7826 | 0.076 | 2.121 | 3.510 | 3.568 | 3.419 |
| KKK-εK-UBITh1 | | 7827 | 0.074 | 3.155 | 4.706 | 3.675 | 3.386 |
| | | 7828 | 0.070 | 3.070 | 4.178 | 3.092 | 2.768 |
| PACAP38₁₋₁₅- | 111 | 7886 | 0.063 | 4.655 | 5.832 | 5.698 | 5.389 |
| KKK-εK-UBITh1 | | 7887 | 0.063 | 3.942 | >10 | 6.285 | 5.568 |
| | | 7888 | 0.063 | 3.835 | 5.858 | 6.264 | 5.124 |
| PACAP38₁₋₂₀- | 112 | 7829 | 0.081 | 5.182 | 8.925 | 5.640 | 5.388 |
| KKK-εK-UBITh1 | | 7830 | 0.081 | 4.867 | 5.670 | 5.240 | 5.122 |
| | | 7831 | 0.069 | 4.917 | >10 | 7.415 | 7.184 |
| PACAP38₆₋₂₀- | 113 | 7904 | 0.068 | 4.566 | 6.298 | 5.266 | 6.580 |
| KKK-εK-UBITh1 | | 7905 | 0.075 | 4.832 | >10 | 6.426 | 5.802 |
| | | 7906 | 0.061 | 5.015 | 5.305 | 4.975 | 4.869 |
| PACAP38₆₋₂₇- | 114 | 7889 | 0.079 | 3.401 | 4.760 | 3.937 | 4.577 |
| KKK-εK-UBITh1 | | 7890 | 0.066 | 4.666 | 5.111 | 4.845 | 5.015 |
| | | 7891 | 0.066 | 4.321 | 4.653 | 4.707 | 4.514 |
| UBITh1-εK- | 115 | 7892 | 0.070 | 4.641 | 5.314 | 5.128 | 4.949 |
| KKK-PACAP38₆₋₂₇ | | 7893 | 0.065 | 4.665 | 6.116 | 5.506 | 5.175 |
| | | 7894 | 0.059 | 4.702 | 5.218 | 5.158 | 8.225 |
| UBITh1-εK- | 116 | 7895 | 0.065 | 3.165 | 3.366 | 2.869 | 2.372 |
| KKK-PACAP38₁₀₋₂₉ | | 7896 | 0.066 | 3.480 | 3.421 | 2.455 | 2.113 |
| | | 7897 | 0.080 | 2.680 | 2.841 | 2.115 | 1.901 |
| UBITh1-εK- | 117 | 7832 | 0.070 | 3.385 | 4.682 | 4.547 | 4.719 |
| KKK-PACAP38₁₃₋₃₄ | | 7833 | 0.073 | 3.319 | 3.960 | 3.393 | 4.607 |
| | | 7834 | 0.063 | 3.260 | 4.699 | 4.840 | 4.836 |
| UBITh1-εK- | 118 | 7907 | 0.060 | 1.297 | 3.119 | 3.282 | 3.428 |
| KKK-PACAP38₁₄₋₂₇ | | 7908 | 0.055 | 0.216 | 2.882 | 2.396 | 1.859 |
| | | 7909 | 0.060 | 0.855 | 3.395 | 3.525 | 3.657 |
| UBITh1-εK- | 119 | 7898 | 0.064 | 2.887 | 4.540 | 4.528 | 4.377 |
| KKK-PACAP38₁₅₋₃₂ | | 7899 | 0.062 | 3.250 | 4.394 | 4.149 | 3.562 |
| | | 7900 | 0.063 | 2.793 | 3.318 | 3.056 | 2.873 |
| UBITh1-εK- | 120 | 7838 | 0.089 | 2.153 | 3.300 | 2.372 | 1.914 |
| KKK-PACAP38₁₇₋₃₀ | | 7839 | 0.089 | 3.343 | 3.703 | 3.285 | 3.018 |
| | | 7840 | 0.075 | 2.385 | 3.519 | 3.196 | 2.944 |
| PACAP38₁₇₋₃₀- | 121 | 7841 | 0.065 | 4.281 | 5.080 | 4.741 | 4.563 |
| KKK-εK-UBITh1 | | 7842 | 0.072 | 0.326 | 4.664 | 4.521 | 4.630 |
| | | 7843 | 0.064 | 3.100 | 4.490 | 4.762 | 4.865 |
| UBITh1-εK- | 122 | 7835 | 0.069 | 2.646 | 3.401 | 2.948 | 3.034 |
| KKK-PACAP38₁₇₋₃₄ | | 7836 | 0.061 | 4.861 | 4.840 | 4.378 | 4.580 |
| | | 7837 | 0.075 | 2.343 | 3.224 | 3.247 | 3.467 |
| UBITh1-εK- | 123 | 7844 | 0.061 | 1.213 | 2.886 | 0.918 | 0.543 |
| KKK-PACAP3₁₈₋₂₈ | | 7845 | 0.083 | 2.220 | 0.326 | 0.930 | 1.441 |
| | | 7846 | 0.084 | 3.044 | 3.237 | 1.881 | 1.292 |
| PACAP38₁₈₋₂₈- | 124 | 7847 | 0.071 | 0.969 | 2.767 | 3.164 | 3.394 |
| KKK-εK-UBITh1 | | 7848 | 0.073 | 0.000 | 4.432 | 4.644 | 4.778 |
| | | 7849 | 0.070 | 0.000 | 2.551 | 0.770 | 1.468 |
| UBITh1-εK- | 125 | 7853 | 0.077 | 1.977 | 4.522 | 4.255 | 4.223 |
| KKK-PACAP38₁₈₋₃₂ | | 7854 | 0.075 | 2.842 | 3.295 | 3.200 | 3.282 |
| | | 7855 | 0.072 | 3.292 | 4.103 | 3.621 | 3.842 |
| UBITh1-εK- | 126 | 7850 | 0.072 | 2.764 | 3.466 | 3.780 | 4.002 |
| KKK-PACAP38₁₈₋₃₅ | | 7851 | 0.073 | 3.197 | 3.964 | 3.869 | 3.911 |
| | | 7852 | 0.074 | 1.704 | 2.672 | 3.248 | 3.409 |
| UBITh1-εK- | 127 | 7856 | 0.074 | 5.104 | >10 | 7.670 | 6.887 |
| KKK-PACAP38₁₈₋₃₈ | | 7857 | 0.063 | 4.731 | >10 | >10 | >10 |
| | | 7858 | 0.062 | 6.035 | >10 | >10 | >10 |
| UBITh1-εK- | 128 | 7859 | 0.085 | 5.173 | >10 | 9.168 | >10 |
| KKK-PACAP38₂₂₋₃₈ | | 7860 | 0.077 | 5.002 | 7.019 | 7.715 | >10 |
| | | 7861 | 0.075 | >10 | 9.456 | 5.868 | 6.320 |
| UBITh1-εK- | 129 | 7862 | 0.082 | >10 | >10 | 7.287 | 8.074 |
| KKK-PACAP38₂₅₋₃₈ | | 7863 | 0.074 | 5.159 | >10 | 7.301 | 8.807 |
| | | 7864 | 0.064 | 5.145 | >10 | 6.468 | 7.121 |
| UBITh1-εK- | 130 | 7910 | 0.066 | 4.846 | >10 | 8.360 | 7.067 |
| KKK-PACAP38₂₈₋₃₈ | | 7911 | 0.067 | 4.502 | 5.673 | 5.636 | 6.347 |
| | | 7912 | 0.074 | 4.950 | >10 | 6.495 | 6.150 |
| UBITh1-εK- | 131 | 7901 | 0.062 | 5.547 | >10 | >10 | 9.610 |
| KKK-PACAP38₃₀₋₃₈ | | 7902 | 0.064 | 5.129 | >10 | 5.569 | 6.531 |
| | | 7903 | 0.063 | >10 | >10 | >10 | >10 |

TABLE 6
Immunogenicity Assessment in Guinea Pigs
of PACAP38 Peptide Immunogen Constructs

| | Anti-PACP38 (Log EC₅₀) | |
|---|---|---|
| SEQ ID NO: | 3 wpi | 6 wpi |
| 110 | 2.72 | 4.07 |
| 111 | 3.4 | 5.1 |
| 112 | 4.28 | 4.72 |
| 113 | 4.1 | 5.24 |
| 114 | 3.53 | 4.34 |
| 115 | 3.86 | 4.8 |
| 116 | 2.46 | 3.33 |
| 117 | 3.23 | 3.3 |
| 118 | BDL | 2.03 |
| 119 | 2.45 | 2.36 |
| 120 | BDL | BDL |
| 121 | BDL | 2.37 |
| 122 | 3.02 | 3.04 |
| 123 | BDL | BDL |
| 124 | BDL | BDL |
| 125 | BDL | 2.79 |
| 126 | BDL | BDL |
| 127 | 4.68 | 5.94 |
| 128 | 4.77 | 5.38 |
| 129 | 4.74 | 5.46 |
| 130 | 3.72 | 5.46 |
| 131 | 5.28 | 5.69 |

TABLE 7
Immunogenicity Assessment in Guinea Pigs against the Th Epitope
Portion of the selected PACAP38 Peptide Immunogen Constructs

| | | | PACAP38₁₋₃₈ | | | UBITh1 | | |
|---|---|---|---|---|---|---|---|---|
| Peptide | | | (SEQ ID NO: 1) | | | (SEQ ID NO: 94) | | |
| immunogen | SEQ ID | Animal | ELISA Log₁₀ Titer | | | ELISA Log₁₀ Titer | | |
| description | NO: | ID | 0 wpi | 3 wpi | 6 wpi | 0 wpi | 3 wpi | 6 wpi |
| PACAP38₁₋₁₅- | 111 | 7886 | 0.063 | 4.655 | 5.832 | 0.053 | 0.000 | 0.221 |
| KKK-εK-UBITh1 | | 7887 | 0.063 | 3.942 | >10 | 0.053 | 0.000 | 0.000 |
| | | 7888 | 0.063 | 3.835 | 5.858 | 0.053 | 0.000 | 0.000 |
| PACAP38₆₋₂₀- | 113 | 7904 | 0.068 | 4.566 | 6.298 | 0.060 | 0.000 | 1.597 |
| KKK-εK-UBITh1 | | 7905 | 0.075 | 4.832 | >10 | 0.066 | 0.000 | 0.000 |
| | | 7906 | 0.061 | 5.015 | 5.305 | 0.061 | 0.000 | 0.000 |

TABLE 8
Mapping of PACAP38 Binding B Epitopes with Immune Sera from PACAP38 Peptide Immunogen Constructs

| | | | A₄₅₀ₙₘ ELISA of Immune Sera (12 wpi) from | | | | | | | |
|---|---|---|---|---|---|---|---|---|---|---|
| | | | PACAP38 Peptide Immunogen Constructs | | | | | | | |
| | | | SEQ ID NO | | | | | | | |
| | | | (PACAP38 amino acids) | | | | | | | |
| 10 mer peptide design for epitope | SEQ ID | Amino | 112 | 115 | 117 | 119 | 122 | 125 | 127 | 128 |
| snapping from −10 to 45 of PACAP38 | NO | Acids | (1-20) | (6-27) | (13-34) | (15-32) | (17-34) | (18-32) | (18-38) | (22-38) |
| GDDAEPLSER | 21 | −10-−1 | 0.111 | 0116 | 2883 | 0.079 | 1.503 | 0.437 | 0.067 | 0.091 |
| DDAEPLSERH | 22 | −9-1 | 0.151 | 0.099 | 0.347 | 0.065 | 0.166 | 0.187 | 0.071 | 0.095 |
| DSEPLSERRS | 23 | −8-2 | 0.146 | 0.093 | 0.154 | 0.053 | 0.126 | 0.206 | 0.068 | 0.071 |
| SEPLSKRHSD | 24 | −7-3 | 0.153 | 0.096 | 0.078 | 0.062 | 0.063 | 0.067 | 0.055 | 0.072 |
| EPLSERRSDG | 25 | −6-4 | 0.166 | 0.119 | 0.079 | 0.056 | 0.055 | 0.053 | 0.067 | 0.071 |
| PLSKRRSDGI | 26 | −5-5 | 0.154 | 0.119 | 0.075 | 0.068 | 0.066 | 0.054 | 0.064 | 0.080 |
| LSKRRSDGIF | 27 | −4-6 | 0.160 | 0.113 | 0.082 | 0.054 | 0.052 | 0.052 | 0.069 | 0.073 |
| SKRRSDGIFT | 28 | −3-7 | 0.134 | 0.103 | 0.069 | 0.064 | 0.063 | 0.060 | 0.065 | 0.071 |
| KRRSDGIFTD | 29 | −2-8 | 0.149 | 0.098 | 0.070 | 0.055 | 0.055 | 0.061 | 0.069 | 0.073 |
| RRSDGIFTDS | 30 | −1-9 | 0.121 | 0.109 | 0.084 | 0.067 | 0.063 | 0.058 | 0.070 | 0.072 |
| RSDGIFTDSY | 31 | 1-10 | 0.196 | 0.125 | 0.107 | 0.082 | 0.076 | 0.085 | 0.095 | 0.091 |
| SDGIFTDSYS | 32 | 2-11 | 0.374 | 0.117 | 0.083 | 0.072 | 0.075 | 0.072 | 0.073 | 0.082 |
| DGIFTDSYSR | 33 | 3-12 | 3.669 | 0.121 | 0.670 | 0.076 | 0.263 | 0.163 | 0.072 | 0.080 |
| GIFTDSYSRY | 34 | 4-13 | 0.243 | 0.126 | 0.061 | 0.053 | 0.064 | 0.055 | 0.073 | 0.088 |
| IFTDSYSRYR | 35 | 5-14 | 0.334 | 0.573 | 0.364 | 0.134 | 0.130 | 4.000 | 0.073 | 0.077 |
| FTDSYSRYRK | 36 | 6-15 | 3.687 | 3.779 | 0.162 | 0.061 | 0.090 | 0.161 | 0.075 | 0.197 |
| TDSYSRYRKQ | 37 | 7-16 | 0.783 | 2.363 | 0.120 | 0.073 | 0.077 | 0.099 | 0.080 | 0.078 |
| DSYSRYRKQM | 38 | 8-17 | 2.903 | 3.746 | 0.265 | 0.078 | 0.297 | 0.095 | 0.101 | 0.100 |
| SYSRYRKQMA | 39 | 9-18 | 0.362 | 3.029 | 0.089 | 0.072 | 0.071 | 0.075 | 0.069 | 0.073 |
| YSRYRKQMAV | 40 | 10-19 | 0.252 | 0.216 | 0.094 | 0.080 | 0.079 | 0.082 | 0.074 | 0.073 |
| SRYRKQMAVK | 41 | 11-20 | 0.752 | 0.104 | 0.204 | 0.102 | 0.126 | 0.281 | 0.033 | 1.756 |
| RYRKQMAVKK | 42 | 12-21 | 0.239 | 0.141 | 0.737 | 0.120 | 0.292 | 0.390 | 0.088 | 3.084 |
| YRKQMAVKKY | 43 | 13-22 | 0.178 | 0.146 | 0.257 | 0.191 | 0.170 | 0.133 | 0.126 | 0.128 |
| RKQMAVKKYL | 44 | 14-23 | 0.117 | 0.097 | 0.121 | 2.416 | 0.076 | 4.000 | 0.094 | 0.097 |
| KQMAVKKYLA | 45 | 15-24 | 0.203 | 0.124 | 0.114 | 0.127 | 0.078 | 0.078 | 0.038 | 0.094 |
| QMAVKKYLAA | 46 | 16-25 | 0.192 | 0.128 | 0.106 | 0.119 | 0.118 | 0.070 | 0.109 | 0.111 |
| MAVKKYLAAV | 47 | 17-26 | 0.202 | 0.156 | 0.122 | 0.096 | 0.082 | 0.066 | 0.088 | 0.081 |
| AVKKYLAAVL | 48 | 18-27 | 0.204 | 0.136 | 0.109 | 0.101 | 0.079 | 0.070 | 0.084 | 0.079 |
| VKKYLAAVLG | 49 | 19-28 | 0.264 | 0.163 | 0.125 | 0.086 | 0.082 | 0.071 | 0.076 | 0.089 |
| KKYLAAVLGK | 50 | 20-29 | 0.213 | 0.147 | 0.108 | 0.089 | 0.083 | 0.079 | 0.090 | 0.089 |
| KYLAAVLGKR | 51 | 21-30 | 0.197 | 0.146 | 0.255 | 0.090 | 0.108 | 0.075 | 0.088 | 0.099 |
| YLAAVLGKRY | 52 | 22-31 | 0.173 | 0.144 | 0.101 | 0.083 | 0.074 | 0.248 | 0.086 | 0.087 |
| LAAVLGKRYK | 53 | 23-32 | 0.187 | 0.144 | 0.204 | 3.863 | 0.118 | 4.000 | 0.125 | 3.332 |
| AAVLGKRYKQ | 54 | 24-33 | 0.180 | 0.157 | 0.158 | 0.120 | 0.135 | 0.490 | 0.120 | 0.189 |
| AVLGKRYKQR | 55 | 25-34 | 0.174 | 0.146 | 3.879 | 0.133 | 4.000 | 0.296 | 4.000 | 3.913 |
| VLGKRYKQRV | 56 | 26-35 | 0.180 | 0.149 | 0.315 | 0.098 | 0.146 | 0.139 | 0.097 | 0.091 |
| LGKRYKQRVK | 57 | 27-36 | 0.237 | 0.144 | 0.539 | 2.360 | 0.408 | 4.000 | 1.400 | 4.000 |
| GKRYKQRVKN | 58 | 28-37 | 0.199 | 0.142 | 0.119 | 0.105 | 0.094 | 0.121 | 2.365 | 3.726 |
| KRYKQRVKNK | 59 | 29-38 | 0.294 | 0.196 | 3.893 | 1.187 | 1.081 | 3.980 | 4.000 | 4.000 |
| RYKQRVKNKG | 60 | 30-39 | 0.179 | 0.159 | 0.114 | 0.098 | 0.092 | 0.091 | 3.519 | 2.428 |
| YKQRVKNKGR | 61 | 31-40 | 0.183 | 0.159 | 3.918 | 0.090 | 4.000 | 0.184 | 1.491 | 1.479 |
| KQRVKNKGRR | 62 | 32-41 | 0.183 | 0.143 | 3.766 | 0.103 | 3.162 | 0.626 | 0.213 | 0.236 |
| QRVKNKGRRI | 63 | 33-42 | 0.199 | 0.148 | 0.167 | 0.085 | 0.105 | 0.106 | 0.202 | 0.259 |
| RVKNKGRRIA | 64 | 34-43 | 0.205 | 0.149 | 0.134 | 0.099 | 0.090 | 0.093 | 0.124 | 0.126 |
| VKNKGRRIAY | 65 | 35-44 | 0.152 | 0.147 | 0.125 | 0.096 | 0.089 | 0.115 | 0.115 | 0.142 |
| KNKGRRIAYL | 66 | 36-45 | 0.154 | 0.133 | 0.109 | 0.090 | 0.082 | 0.374 | 0.160 | 0.183 |
| HSDGIFTDSYSRYRKQMAVKKYLAAVLGKRYKQRVKNK | 1 | 1-38 | 3.688 | 3.194 | 1.187 | 0.262 | 0.300 | 0.332 | 3.880 | 3.955 |
| GDDAEPLSKRHSDGIFTDSYSRYRKQMAVKKYLAAVLGKRYKQRVKNKGRRIAYL | 197 | | | | | | | | | |

1. A PACAP peptide immunogen construct having about 20 or more amino acids, represented by the formulae:
(Th)ₘ-(A)ₙ-(PACAP functional B cell epitope peptide)-X
or
(PACAP functional B cell epitope peptide)-(A)ₙ-(Th)ₘ-X
or
(Th)ₘ-(A)ₙ-(PACAP functional B cell epitope peptide)-(A)ₙ-(Th)ₘ-X
wherein
Th is a heterologous T helper epitope;
A is a heterologous spacer;
(PACAP functional B cell epitope peptide) is a B cell epitope peptide having from 9 to about 22 amino acid residues of PACAP (SEQ ID NO: 1);
X is an α-COOH or α-CONH₂ of an amino acid;
m is from 1 to about 4; and
n is from 0 to about 10.

2. The PACAP peptide immunogen construct according to claim 1, wherein the PACAP functional B cell epitope peptide is selected from the group consisting of SEQ ID NOs: 2-20.

3. The PACAP peptide immunogen construct according to claim 1, wherein the Th epitope is selected from the group consisting of SEQ ID NOs: 70-109 and 106-171.

4. The PACAP peptide immunogen construct according to claim 1, wherein the PACAP functional B cell epitope peptide is selected from the group consisting of SEQ ID NOs: 2-20 and the Th epitope is selected from the group consisting of SEQ ID NOs: 70-109 and 106-171.

5. The PACAP peptide immunogen construct according to claim 1, wherein the peptide immunogen construct is selected from the group consisting of SEQ ID NOs: 110-159.

6. An PACAP peptide immunogen construct comprising:
a. a B cell epitope comprising from about 9 to about 22 amino acid residues from the PACAP38 sequence of SEQ ID NO: 1;
b. a T helper epitope comprising an amino acid sequence selected from the group consisting of SEQ ID NOs: 70-109 and 160-171, and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 68), Lys-Lys-Lys-ε-N-Lys (SEQ ID NO: 69), and Pro-Pro-Xaa-Pro-Xaa-Pro (SEQ ID NO: 67), and any combination thereof,
wherein the B cell epitope is covalently linked to the T helper epitope directly or through the optional heterologous spacer.

7. The PACAP peptide immunogen construct of claim 6, wherein the B cell epitope is selected from the group consisting of SEQ ID NOs: 2-20.

8. The PACAP peptide immunogen construct of claim 6, wherein the optional heterologous spacer is (α, ε-N)Lys, ε-N-Lys-Lys-Lys-Lys (SEQ ID NO: 68), Lys-Lys-Lys-ε-N-Lys (SEQ ID NO: 69), or Pro-Pro-Xaa-Pro-Xaa-Pro (SEQ ID NO: 67), where Xaa is any amino acid.

9. The PACAP peptide immunogen construct of claim 6, wherein the T helper epitope is covalently linked to the amino- or carboxyl-terminus of the B cell epitope.

10. The PACAP peptide immunogen construct of claim 6, wherein the T helper epitope is covalently linked to the amino- or carboxyl-terminus of the B cell epitope through the optional heterologous spacer.

11. A composition comprising the PACAP peptide immunogen construct according to claim 1.

12. A pharmaceutical composition comprising:
a. a peptide immunogen construct according to claim 1; and
b. a pharmaceutically acceptable delivery vehicle and/or adjuvant.

13. The pharmaceutical composition of claim 12, wherein
a. the PACAP functional B cell epitope peptide is selected from the group consisting of SEQ ID NO: 2-20;
b. the Th epitope is selected from the group consisting of SEQ ID NOs: 70-109 and 160-171; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 68), Lys-Lys-Lys-ε-N-Lys (SEQ ID NO: 69), and Pro-Pro-Xaa-Pro-Xaa-Pro (SEQ ID NO:67), and any combination thereof; and
wherein the PACAP peptide immunogen construct is mixed with an CpG oligodeoxynucleotide (ODN) to form a stabilized immunostimulatory complex.

14. The pharmaceutical composition of claim 12, wherein
a. the PACAP peptide immunogen construct is selected from the group consisting of SEQ ID NOs: 110-131 and 132-159; and
wherein the PACAP peptide immunogen construct is mixed with an CpG oligodeoxynucleotide (ODN) to form a stabilized immunostimulatory complex.

15. A method for generating antibodies against PACAP in an animal comprising administering the pharmaceutical composition according to claim 12 to the animal.

16. An isolated antibody or epitope-binding fragment thereof that specifically binds to the PAC1 binding or activation region of SEQ ID NOs: 2-20.

17. The isolated antibody or epitope-binding fragment thereof according to claim 16 bound to the PACAP peptide immunogen construct.

18. A composition comprising the isolated antibody or epitope-binding fragment thereof according to claim 16.

19. A method of preventing and/or treating migraine in an animal comprising administering the pharmaceutical composition of claim 16 to the animal.


SEQUENCE LISTING (197 sequences)

SEQ ID NO: 1 - protein, 38 aa, Homo sapiens
  PEPTIDE (1)..(38): Human/Rat/Mouse PACAP38
HSDGIFTDSYSRYRKQMAVKKYLAAVLGKRYKQRVKNK

SEQ ID NO: 2 - protein, 10 aa, Homo sapiens
  PEPTIDE (1)..(10): PACAP38 (1-10)
HSDGIFTDSY

SEQ ID NO: 3 - protein, 15 aa, Homo sapiens
  PEPTIDE (1)..(15): PACAP38 (1-15)
HSDGIFTDSYSRYRK

SEQ ID NO: 4 - protein, 20 aa, Homo sapiens
  PEPTIDE (1)..(20): PACAP38 (1-20)
HSDGIFTDSYSRYRKQMAVK

SEQ ID NO: 5 - protein, 15 aa, Homo sapiens
  PEPTIDE (1)..(15): PACAP38 (6-20)
FTDSYSRYRKQMAVK

SEQ ID NO: 6 - protein, 22 aa, Homo sapiens
  PEPTIDE (1)..(22): PACAP38 (6-27)
FTDSYSRYRKQMAVKKYLAAVL

SEQ ID NO: 7 - protein, 20 aa, Homo sapiens
  PEPTIDE (1)..(20): PACAP38 (10-29)
YSRYRKQMAVKKYLAAVLGK

SEQ ID NO: 8 - protein, 22 aa, Homo sapiens
  PEPTIDE (1)..(22): PACAP38 (13-34)
YRKQMAVKKYLAAVLGKRYKQR

SEQ ID NO: 9 - protein, 14 aa, Homo sapiens
  PEPTIDE (1)..(14): PACAP38 (14-27)
RKQMAVKKYLAAVL

SEQ ID NO: 10 - protein, 18 aa, Homo sapiens
  PEPTIDE (1)..(18): PACAP38 (15-32)
KQMAVKKYLAAVLGKRYK

SEQ ID NO: 11 - protein, 14 aa, Homo sapiens
  PEPTIDE (1)..(14): PACAP38 (17-30)
MAVKKYLAAVLGKR

SEQ ID NO: 12 - protein, 18 aa, Homo sapiens
  PEPTIDE (1)..(18): PACAP38 (17-34)
MAVKKYLAAVLGKRYKQR

SEQ ID NO: 13 - protein, 11 aa, Homo sapiens
  PEPTIDE (1)..(11): PACAP38 (18-28)
AVKKYLAAVLG

SEQ ID NO: 14 - protein, 15 aa, Homo sapiens
  PEPTIDE (1)..(15): PACAP38 (18-32)
AVKKYLAAVLGKRYK

SEQ ID NO: 15 - protein, 18 aa, Homo sapiens
  PEPTIDE (1)..(18): PACAP38 (18-35)
AVKKYLAAVLGKRYKQRV

SEQ ID NO: 16 - protein, 21 aa, Homo sapiens
  PEPTIDE (1)..(21): PACAP38 (18-38)
AVKKYLAAVLGKRYKQRVKNK

SEQ ID NO: 17 - protein, 17 aa, Rattus norvegicus
  PEPTIDE (1)..(17): PACAP38 (22-38)
YLAAVLGKRYKQRVKNK

SEQ ID NO: 18 - protein, 14 aa, Homo sapiens
  PEPTIDE (1)..(14): PACAP38 (25-38)
AVLGKRYKQRVKNK

SEQ ID NO: 19 - protein, 11 aa, Homo sapiens
  PEPTIDE (1)..(11): PACAP38 (28-38)
GKRYKQRVKNK

SEQ ID NO: 20 - protein, 9 aa, Homo sapiens
  PEPTIDE (1)..(9): PACAP38 (30-38)
RYKQRVKNK

SEQ ID NO: 21 - protein, 10 aa, Homo sapiens
  PEPTIDE (1)..(10): PACAP38 (-10 - -1)
GDDAEPLSKR

SEQ ID NO: 22 - protein, 10 aa, Homo sapiens
  PEPTIDE (1)..(10): PACAP38 (-9 - 1)
DDAEPLSKRH

SEQ ID NO: 23 - protein, 10 aa, Homo sapiens
  PEPTIDE (1)..(10): PACAP38 (-8 - 2)
DSEPLSKRHS

SEQ ID NO: 24 - protein, 10 aa, Homo sapiens
  PEPTIDE (1)..(10): PACAP38 (-7 - 3)
SEPLSKRHSD

SEQ ID NO: 25 - protein, 10 aa, Homo sapiens
  PEPTIDE (1)..(10): PACAP38 (-6 - 4)
EPLSKRHSDG

SEQ ID NO: 26 - protein, 10 aa, Homo sapiens
  PEPTIDE (1)..(10): PACAP38 (-5 - 5)
PLSKRHSDGI

SEQ ID NO: 27 - protein, 10 aa, Homo sapiens
  PEPTIDE (1)..(10): PACAP38 (-4 - 6)
LSKRHSDGIF

SEQ ID NO: 28 - protein, 10 aa, Homo sapiens
  PEPTIDE (1)..(10): PACAP38 (-3 - 7)
SKRHSDGIFT

SEQ ID NO: 29 - protein, 10 aa, Homo sapiens
  PEPTIDE (1)..(10): PACAP38 (-2 - 8)
KRHSDGIFTD

SEQ ID NO: 30 - protein, 10 aa, Homo sapiens
  PEPTIDE (1)..(10): PACAP38 (-1 - 9)
RHSDGIFTDS

SEQ ID NO: 31 - protein, 10 aa, Homo sapiens
  PEPTIDE (1)..(10): PACAP38 (1 - 10)
HSDGIFTDSY

SEQ ID NO: 32 - protein, 10 aa, Homo sapiens
  PEPTIDE (1)..(10): PACAP38 (2 - 11)
SDGIFTDSYS

SEQ ID NO: 33 - protein, 10 aa, Homo sapiens
  PEPTIDE (1)..(10): PACAP38 (3 - 12)
DGIFTDSYSR

SEQ ID NO: 34 - protein, 10 aa, Homo sapiens
  PEPTIDE (1)..(10): PACAP38 (4 - 13)
GIFTDSYSRY

SEQ ID NO: 35 - protein, 10 aa, Homo sapiens
  PEPTIDE (1)..(10): PACAP38 (5 - 14)
IFTDSYSRYR

SEQ ID NO: 36 - protein, 10 aa, Homo sapiens
  PEPTIDE (1)..(10): PACAP38 (6 - 15)
FTDSYSRYRK

SEQ ID NO: 37 - protein, 10 aa, Homo sapiens
  PEPTIDE (1)..(10): PACAP38 (7 - 16)
TDSYSRYRKQ

SEQ ID NO: 38 - protein, 10 aa, Homo sapiens
  PEPTIDE (1)..(10): PACAP38 (8 - 17)
DSYSRYRKQM

SEQ ID NO: 39 - protein, 10 aa, Homo sapiens
  PEPTIDE (1)..(10): PACAP38 (9 - 18)
SYSRYRKQMA

SEQ ID NO: 40 - protein, 10 aa, Homo sapiens
  PEPTIDE (1)..(10): PACAP38 (10 - 19)
YSRYRKQMAV

SEQ ID NO: 41 - protein, 10 aa, Homo sapiens
  PEPTIDE (1)..(10): PACAP38 (11 - 20)
SRYRKQMAVK

SEQ ID NO: 42 - protein, 10 aa, Homo sapiens
  PEPTIDE (1)..(10): PACAP38 (12 - 21)
RYRKQMAVKK

SEQ ID NO: 43 - protein, 10 aa, Homo sapiens
  PEPTIDE (1)..(10): PACAP38 (13 - 22)
YRKQMAVKKY

SEQ ID NO: 44 - protein, 10 aa, Homo sapiens
  PEPTIDE (1)..(10): PACAP38 (14 - 23)
RKQMAVKKYL

SEQ ID NO: 45 - protein, 10 aa, Homo sapiens
  PEPTIDE (1)..(10): PACAP38 (15 - 24)
KQMAVKKYLA

SEQ ID NO: 46 - protein, 10 aa, Homo sapiens
  PEPTIDE (1)..(10): PACAP38 (16 - 25)
QMAVKKYLAA

SEQ ID NO: 47 - protein, 10 aa, Homo sapiens
  PEPTIDE (1)..(10): PACAP38 (17 - 26)
MAVKKYLAAV

SEQ ID NO: 48 - protein, 10 aa, Homo sapiens
  PEPTIDE (1)..(10): PACAP38 (18 - 27)
AVKKYLAAVL

SEQ ID NO: 49 - protein, 10 aa, Homo sapiens
  PEPTIDE (1)..(10): PACAP38 (19 - 28)
VKKYLAAVLG

SEQ ID NO: 50 - protein, 10 aa, Homo sapiens
  PEPTIDE (1)..(10): PACAP38 (20 - 29)
KKYLAAVLGK

SEQ ID NO: 51 - protein, 10 aa, Homo sapiens
  PEPTIDE (1)..(10): PACAP38 (21 - 30)
KYLAAVLGKR

SEQ ID NO: 52 - protein, 10 aa, Homo sapiens
  PEPTIDE (1)..(10): PACAP38 (22 - 31)
YLAAVLGKRY

SEQ ID NO: 53 - protein, 10 aa, Homo sapiens
  PEPTIDE (1)..(10): PACAP38 (23 - 32)
LAAVLGKRYK

SEQ ID NO: 54 - protein, 10 aa, Homo sapiens
  PEPTIDE (1)..(10): PACAP38 (24 - 33)
AAVLGKRYKQ

SEQ ID NO: 55 - protein, 10 aa, Homo sapiens
  PEPTIDE (1)..(10): PACAP38 (25 - 34)
AVLGKRYKQR

SEQ ID NO: 56 - protein, 10 aa, Homo sapiens
  PEPTIDE (1)..(10): PACAP38 (26 - 35)
VLGKRYKQRV

SEQ ID NO: 57 - protein, 10 aa, Homo sapiens
  PEPTIDE (1)..(10): PACAP38 (27 - 36)
LGKRYKQRVK

SEQ ID NO: 58 - protein, 10 aa, Homo sapiens
  PEPTIDE (1)..(10): PACAP38 (28 - 37)
GKRYKQRVKN

SEQ ID NO: 59 - protein, 10 aa, Homo sapiens
  PEPTIDE (1)..(10): PACAP38 (29 - 38)
KRYKQRVKNK

SEQ ID NO: 60 - protein, 10 aa, Homo sapiens
  PEPTIDE (1)..(10): PACAP38 (30 - 39)
RYKQRVKNKG

SEQ ID NO: 61 - protein, 10 aa, Homo sapiens
  PEPTIDE (1)..(10): PACAP38 (31 - 40)
YKQRVKNKGR

SEQ ID NO: 62 - protein, 10 aa, Homo sapiens
  PEPTIDE (1)..(10): PACAP38 (32 - 41)
KQRVKNKGRR

SEQ ID NO: 63 - protein, 10 aa, Homo sapiens
  PEPTIDE (1)..(10): PACAP38 (33 - 42)
QRVKNKGRRI

SEQ ID NO: 64 - protein, 10 aa, Homo sapiens
  PEPTIDE (1)..(10): PACAP38 (34 - 43)
RVKNKGRRIA

SEQ ID NO: 65 - protein, 10 aa, Homo sapiens
  PEPTIDE (1)..(10): PACAP38 (35 - 44)
VKNKGRRIAY

SEQ ID NO: 66 - protein, 10 aa, Homo sapiens
  PEPTIDE (1)..(10): PACAP38 (36 - 45)
KNKGRRIAYL

SEQ ID NO: 67 - protein, 6 aa, Artificial Sequence
  Synthetic peptide
  PEPTIDE (1)..(6): Flexible Hinge Spacer
  SITE (3)..(3): Xaa is any amino acid, preferably Asp
  SITE (5)..(5): Xaa is any amino acid, preferably Asp
PPXPXP

SEQ ID NO: 68 - protein, 4 aa, Artificial Sequence
  Synthetic peptide
  SITE (1)..(1): epsilon-K
  PEPTIDE (1)..(4): epsilon-K-KKK as a spacer
KKKK

SEQ ID NO: 69 - protein, 4 aa, Artificial Sequence
  Synthetic peptide
  PEPTIDE (1)..(4): KKK-epsilon-K as a spacer
  SITE (4)..(4): epsilon-K
KKKK

SEQ ID NO: 70 - protein, 17 aa, Clostridium tetani
  PEPTIDE (1)..(17): Clostridium tetani 1 Th
KKQYIKANSKFIGITEL

SEQ ID NO: 71 - protein, 15 aa, Measles virus
  PEPTIDE (1)..(15): MvF1 Th
LSEIKGVIVHRLEGV

SEQ ID NO: 72 - protein, 24 aa, Bordetella pertussis
  PEPTIDE (1)..(24): Bordetella pertussis Th
GAYARCPNGTRALTVAELRGNAEL

SEQ ID NO: 73 - protein, 17 aa, Clostridium tetani
  PEPTIDE (1)..(17): Clostridium tetani 2 Th
WVRDIIDDFTNESSQKT

SEQ ID NO: 74 - protein, 23 aa, diphtheria bacilli
  PEPTIDE (1)..(23): Diphtheria Th
DSETADNLEKTVAALSILPGHGC

SEQ ID NO: 75 - protein, 21 aa, Plasmodium falciparum
  PEPTIDE (1)..(21): Plasmodium falciparum Th
DHEKKHAKMEKASSVFNVVNS

SEQ ID NO: 76 - protein, 17 aa, Schistosoma mansoni
  PEPTIDE (1)..(17): Schistosoma mansoni Th
KWFKTNAPNGVDEKHRH

SEQ ID NO: 77 - protein, 25 aa, Cholera Toxin
  PEPTIDE (1)..(25): Cholera Toxin Th
ALNIWDRFDVFCTLGATTGYLKGNS

SEQ ID NO: 78 - protein, 15 aa, Measles virus
  PEPTIDE (1)..(15): MvF 2 Th
ISEIKGVIVHKIEGI

SEQ ID NO: 79 - protein, 22 aa, Measles virus
  PEPTIDE (1)..(22): KKKMvF3 Th
KKKISISEIKGVIVHKIEGILF

SEQ ID NO: 80 - protein, 22 aa, Measles virus
  PEPTIDE (1)..(22): KKKMvF3 Th
KKKISITEIRTVIVTRIETILF

SEQ ID NO: 81 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 82 - protein, 18 aa, Hepatitis B virus
  PEPTIDE (1)..(18): HBsAg1 Th
KKKLFLLTKLLTLPQSLD

SEQ ID NO: 83 - protein, 18 aa, Hepatitis B virus
  PEPTIDE (1)..(18): HBsAg1 Th
RRRIKIITRIITIPLSIR

SEQ ID NO: 84 - protein, 18 aa, Hepatitis B virus
  PEPTIDE (1)..(18): HBsAg1 Th
KKKVRVVTKVVTVPISVD

SEQ ID NO: 85 - protein, 18 aa, Hepatitis B virus
  PEPTIDE (1)..(18): HBsAg1 Th
KKKFFFFTKFFTFPVSFD

SEQ ID NO: 86 - protein, 18 aa, Hepatitis B virus
  PEPTIDE (1)..(18): HBsAg1 Th
KKKLFLLTKLLTLPFSLD

SEQ ID NO: 87 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 88 - protein, 19 aa, Measles virus
  PEPTIDE (1)..(19): MvF4 Th
ISISEIKGVIVHKIETILF

SEQ ID NO: 89 - protein, 19 aa, Measles virus
  PEPTIDE (1)..(19): MvF4 Th
ISITEIRTVIVTRIETILF

SEQ ID NO: 90 - protein, 19 aa, Measles virus
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 91 - protein, 18 aa, Hepatitis B virus
  PEPTIDE (1)..(18): HBsAg2 Th
KKKIITITRIITIPQSLD

SEQ ID NO: 92 - protein, 18 aa, Hepatitis B virus
  PEPTIDE (1)..(18): HBsAg2 Th
KKKFFLLTRILTIITTID

SEQ ID NO: 93 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 94 - protein, 19 aa, Measles virus
  PEPTIDE (1)..(19): MvF5 Th (UBITh1)
ISITEIKGVIVHRIETILF

SEQ ID NO: 95 - protein, 18 aa, Hepatitis B virus
  PEPTIDE (1)..(18): HBsAg 3 Th
KKKIITITRIITIITTID

SEQ ID NO: 96 - protein, 11 aa, Influenza virus
  PEPTIDE (1)..(11): Influenza Matrix protein 1_1 Th
FVFTLTVPSER

SEQ ID NO: 97 - protein, 15 aa, Influenza virus
  PEPTIDE (1)..(15): Influenza Matrix protein 1_2 Th
SGPLKAEIAQRLEDV

SEQ ID NO: 98 - protein, 9 aa, Influenza virus
  PEPTIDE (1)..(9): Influenza Non-structural protein 1 Th
DRLRRDQKS

SEQ ID NO: 99 - protein, 19 aa, Epstein-Barr virus
  PEPTIDE (1)..(19): EBV BHRF1 Th
AGLTLSLLVICSYLFISRG

SEQ ID NO: 100 - protein, 15 aa, Clostridium tetani
  PEPTIDE (1)..(15): Clostridium tetani TT1 Th
QYIKANSKFIGITEL

SEQ ID NO: 101 - protein, 20 aa, Epstein-Barr virus
  PEPTIDE (1)..(20): EBV EBNA-1 Th
PGPLRESIVCYFMVFLQTHI

SEQ ID NO: 102 - protein, 21 aa, Clostridium tetani
  PEPTIDE (1)..(21): Clostridium tetani  TT2 Th
FNNFTVSFWLRVPKVSASHLE

SEQ ID NO: 103 - protein, 16 aa, Clostridium tetani
  PEPTIDE (1)..(16): Clostridium tetani TT3 Th
KFIIKRYTPNNEIDSF

SEQ ID NO: 104 - protein, 16 aa, Clostridium tetani
  PEPTIDE (1)..(16): Clostridium tetani  TT4 Th
VSIDKFRIFCKALNPK

SEQ ID NO: 105 - protein, 18 aa, Epstein-Barr virus
  PEPTIDE (1)..(18): EBV CP Th
VPGLYSPCRAFFNKEELL

SEQ ID NO: 106 - protein, 14 aa, Human cytomegalovirus
  PEPTIDE (1)..(14): HCMV IE1 Th
DKREMWMACIKELH

SEQ ID NO: 107 - protein, 15 aa, Epstein-Barr virus
  PEPTIDE (1)..(15): EBV GP340 Th
TGHGARTSTEPTTDY

SEQ ID NO: 108 - protein, 13 aa, Epstein-Barr virus
  PEPTIDE (1)..(13): EBV BPLF1 Th
KELKRQYEKKLRQ

SEQ ID NO: 109 - protein, 11 aa, Epstein-Barr virus
  PEPTIDE (1)..(11): EBV EBNA-2 Th
TVFYNIPPMPL

SEQ ID NO: 110 - protein, 33 aa, Artificial Sequence
  Synthetic peptide
  PEPTIDE (1)..(10): PACAP38 (1-10)
  PEPTIDE (11)..(14): KKK-epsilon K as a spacer
  SITE (14)..(14): epsilon K
  PEPTIDE (15)..(33): MvF5 Th (UBITh1)
HSDGIFTDSYKKKKISITEIKGVIVHRIETILF

SEQ ID NO: 111 - protein, 38 aa, Artificial Sequence
  Synthetic peptide
  PEPTIDE (1)..(15): PACAP38 (1-15)
  PEPTIDE (16)..(19): KKK-epsilon K as a spacer
  SITE (19)..(19): epsilon K
  PEPTIDE (20)..(38): MvF5 Th (UBITh1)
HSDGIFTDSYSRYRKKKKKISITEIKGVIVHRIETILF

SEQ ID NO: 112 - protein, 43 aa, Artificial Sequence
  Synthetic peptide
  PEPTIDE (1)..(20): PACAP38 (1-20)
  PEPTIDE (21)..(24): KKK-epsilon K as a spacer
  SITE (24)..(24): epsilon K
  PEPTIDE (25)..(43): MvF5 Th (UBITh1)
HSDGIFTDSYSRYRKQMAVKKKKKISITEIKGVIVHRIETILF

SEQ ID NO: 113 - protein, 38 aa, Artificial Sequence
  Synthetic peptide
  PEPTIDE (1)..(15): PACAP38 (6-20)
  PEPTIDE (16)..(19): KKK-epsilon K as a spacer
  SITE (19)..(19): epsilon K
  PEPTIDE (20)..(38): MvF5 Th (UBITh1)
FTDSYSRYRKQMAVKKKKKISITEIKGVIVHRIETILF

SEQ ID NO: 114 - protein, 45 aa, Artificial Sequence
  Synthetic peptide
  PEPTIDE (1)..(22): PACAP38 (6-27)
  PEPTIDE (23)..(26): KKK-epsilon K as a spacer
  SITE (26)..(26): epsilon K
  PEPTIDE (27)..(45): MvF5 Th (UBITh1)
FTDSYSRYRKQMAVKKYLAAVLKKKKISITEIKGVIVHRIETILF

SEQ ID NO: 115 - protein, 45 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(45): PACAP38 (6-27)
ISITEIKGVIVHRIETILFKKKKFTDSYSRYRKQMAVKKYLAAVL

SEQ ID NO: 116 - protein, 43 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): PACAP38 (10-29)
ISITEIKGVIVHRIETILFKKKKYSRYRKQMAVKKYLAAVLGK

SEQ ID NO: 117 - protein, 45 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(45): PACAP38 (13-34)
ISITEIKGVIVHRIETILFKKKKYRKQMAVKKYLAAVLGKRYKQR

SEQ ID NO: 118 - protein, 37 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(37): PACAP38 (14-27)
ISITEIKGVIVHRIETILFKKKKRKQMAVKKYLAAVL

SEQ ID NO: 119 - protein, 41 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(41): PACAP38 (15-32)
ISITEIKGVIVHRIETILFKKKKKQMAVKKYLAAVLGKRYK

SEQ ID NO: 120 - protein, 37 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(37): PACAP38 (17-30)
ISITEIKGVIVHRIETILFKKKKMAVKKYLAAVLGKR

SEQ ID NO: 121 - protein, 37 aa, Artificial Sequence
  Synthetic peptide
  PEPTIDE (1)..(14): PACAP38 (17-30)
  PEPTIDE (15)..(18): KKK-epsilon K as a spacer
  SITE (18)..(18): epsilon K
  PEPTIDE (19)..(37): MvF5 Th (UBITh1)
MAVKKYLAAVLGKRKKKKISITEIKGVIVHRIETILF

SEQ ID NO: 122 - protein, 41 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(41): PACAP38 (17-34)
ISITEIKGVIVHRIETILFKKKKMAVKKYLAAVLGKRYKQR

SEQ ID NO: 123 - protein, 34 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(34): PACAP38 (18-28)
ISITEIKGVIVHRIETILFKKKKAVKKYLAAVLG

SEQ ID NO: 124 - protein, 34 aa, Artificial Sequence
  Synthetic peptide
  PEPTIDE (1)..(11): PACAP38 (18-28)
  PEPTIDE (12)..(15): KKK-epsilon K as a spacer
  SITE (15)..(15): epsilon K
  PEPTIDE (16)..(34): MvF5 Th (UBITh1)
AVKKYLAAVLGKKKKISITEIKGVIVHRIETILF

SEQ ID NO: 125 - protein, 38 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(38): PACAP38 (18-32)
ISITEIKGVIVHRIETILFKKKKAVKKYLAAVLGKRYK

SEQ ID NO: 126 - protein, 41 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(41): PACAP38 (18-35)
ISITEIKGVIVHRIETILFKKKKAVKKYLAAVLGKRYKQRV

SEQ ID NO: 127 - protein, 44 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(44): PACAP38 (18-38)
ISITEIKGVIVHRIETILFKKKKAVKKYLAAVLGKRYKQRVKNK

SEQ ID NO: 128 - protein, 40 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(40): PACAP38 (22-38)
ISITEIKGVIVHRIETILFKKKKYLAAVLGKRYKQRVKNK

SEQ ID NO: 129 - protein, 37 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(37): PACAP38 (25-38)
ISITEIKGVIVHRIETILFKKKKAVLGKRYKQRVKNK

SEQ ID NO: 130 - protein, 34 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(34): PACAP38 (28-38)
ISITEIKGVIVHRIETILFKKKKGKRYKQRVKNK

SEQ ID NO: 131 - protein, 32 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(32): PACAP38 (30-38)
ISITEIKGVIVHRIETILFKKKKRYKQRVKNK

SEQ ID NO: 132 - protein, 42 aa, Artificial Sequence
  Synthetic peptide
  PEPTIDE (1)..(17): Clostridium tetani1 Th
  PEPTIDE (18)..(21): epsilon-K-KKK spacer
  SITE (18)..(18): epsilon-K
  PEPTIDE (22)..(42): PACAP38 (18-38)
KKQYIKANSKFIGITELKKKKAVKKYLAAVLGKRYKQRVKNK

SEQ ID NO: 133 - protein, 40 aa, Artificial Sequence
  Synthetic peptide
  PEPTIDE (1)..(15): MvF1 Th
  PEPTIDE (16)..(19): epsilon-K-KKK spacer
  SITE (16)..(16): epsilon-K
  PEPTIDE (20)..(40): PACAP38 (18-38)
LSEIKGVIVHRLEGVKKKKAVKKYLAAVLGKRYKQRVKNK

SEQ ID NO: 134 - protein, 49 aa, Artificial Sequence
  Synthetic peptide
  PEPTIDE (1)..(24): Bordetella pertussis Th
  PEPTIDE (25)..(28): epsilon-K-KKK spacer
  SITE (25)..(25): epsilon-K
  PEPTIDE (29)..(49): PACAP38 (18-38)
GAYARCPNGTRALTVAELRGNAELKKKKAVKKYLAAVLGKRYKQRVKNK

SEQ ID NO: 135 - protein, 42 aa, Artificial Sequence
  Synthetic peptide
  PEPTIDE (1)..(17): Clostridium tetani2 Th
  PEPTIDE (18)..(21): epsilon-K-KKK spacer
  SITE (18)..(18): epsilon-K
  PEPTIDE (22)..(42): PACAP38 (18-38)
WVRDIIDDFTNESSQKTKKKKAVKKYLAAVLGKRYKQRVKNK

SEQ ID NO: 136 - protein, 48 aa, Artificial Sequence
  Synthetic peptide
  PEPTIDE (1)..(23): Diphtheria Th
  PEPTIDE (24)..(27): epsilon-K-KKK spacer
  SITE (24)..(24): epsilon-K
  PEPTIDE (28)..(48): PACAP38 (18-38)
DSETADNLEKTVAALSILPGHGCKKKKAVKKYLAAVLGKRYKQRVKNK

SEQ ID NO: 137 - protein, 46 aa, Artificial Sequence
  Synthetic peptide
  PEPTIDE (1)..(21): Plasmodium falciparum Th
  PEPTIDE (22)..(25): epsilon-K-KKK spacer
  SITE (22)..(22): epsilon-K
  PEPTIDE (26)..(46): PACAP38 (18-38)
DHEKKHAKMEKASSVFNVVNSKKKKAVKKYLAAVLGKRYKQRVKNK

SEQ ID NO: 138 - protein, 42 aa, Artificial Sequence
  Synthetic peptide
  PEPTIDE (1)..(17): Schistosoma mansoni Th
  PEPTIDE (18)..(21): epsilon-K-KKK spacer
  SITE (18)..(18): epsilon-K
  PEPTIDE (22)..(42): PACAP38 (18-38)
KWFKTNAPNGVDEKHRHKKKKAVKKYLAAVLGKRYKQRVKNK

SEQ ID NO: 139 - protein, 50 aa, Artificial Sequence
  Synthetic peptide
  PEPTIDE (1)..(25): Cholera Toxin Th
  PEPTIDE (26)..(29): epsilon-K-KKK spacer
  SITE (26)..(26): epsilon-K
  PEPTIDE (30)..(50): PACAP38 (18-38)
ALNIWDRFDVFCTLGATTGYLKGNSKKKKAVKKYLAAVLGKRYKQRVKNK

SEQ ID NO: 140 - protein, 40 aa, Artificial Sequence
  Synthetic peptide
  PEPTIDE (1)..(15): MvF2 Th
  PEPTIDE (16)..(19): epsilon-K-KKK spacer
  SITE (16)..(16): epsilon-K
  PEPTIDE (20)..(40): PACAP38 (18-38)
ISEIKGVIVHKIEGIKKKKAVKKYLAAVLGKRYKQRVKNK

SEQ ID NO: 141 - protein, 47 aa, Artificial Sequence
  Synthetic peptide
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  SITE (23)..(23): epsilon K as a spacer
  PEPTIDE (23)..(26): epsilon-K-KKK spacer
  PEPTIDE (27)..(47): PACAP38 (18-38)
KKKISIXEIXXVIVXXIEXILFKKKKAVKKYLAAVLGKRYKQRVKNK

SEQ ID NO: 142 - protein, 43 aa, Artificial Sequence
  Synthetic peptide
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  SITE (19)..(19): epsilon K as a spacer
  PEPTIDE (19)..(22): epsilon-K-KKK spacer
  PEPTIDE (23)..(43): PACAP38 (18-38)
XXXXXXXTXXXTXPXSXXKKKKAVKKYLAAVLGKRYKQRVKNK

SEQ ID NO: 143 - protein, 44 aa, Artificial Sequence
  Synthetic peptide
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon-K-KKK spacer
  PEPTIDE (24)..(44): PACAP38 (18-38)
ISIXEIXXVIVXXIETILFKKKKAVKKYLAAVLGKRYKQRVKNK

SEQ ID NO: 144 - protein, 43 aa, Artificial Sequence
  Synthetic peptide
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  SITE (19)..(19): epsilon K as a spacer
  PEPTIDE (19)..(22): epsilon-K-KKK spacer
  PEPTIDE (23)..(43): PACAP38 (18-38)
KKKXXXXTRIXTIXXXXDKKKKAVKKYLAAVLGKRYKQRVKNK

SEQ ID NO: 145 - protein, 43 aa, Artificial Sequence
  Synthetic peptide
  PEPTIDE (1)..(18): HBsAg3 Th (UBITh2)
  SITE (19)..(19): epsilon-K
  PEPTIDE (19)..(22): epsilon-K-KKK spacer
  PEPTIDE (23)..(43): PACAP38 (18-38)
KKKIITITRIITIITTIDKKKKAVKKYLAAVLGKRYKQRVKNK

SEQ ID NO: 146 - protein, 36 aa, Artificial Sequence
  Synthetic peptide
  PEPTIDE (1)..(11): Influenza MP1_1 Th
  PEPTIDE (12)..(15): epsilon-K-KKK spacer
  SITE (12)..(12): epsilon-K
  PEPTIDE (16)..(36): PACAP38 (18-38)
FVFTLTVPSERKKKKAVKKYLAAVLGKRYKQRVKNK

SEQ ID NO: 147 - protein, 40 aa, Artificial Sequence
  Synthetic peptide
  PEPTIDE (1)..(15): Influenza MP1_2 Th
  PEPTIDE (16)..(19): epsilon-K-KKK spacer
  SITE (16)..(16): epsilon-K
  PEPTIDE (20)..(40): PACAP38 (18-38)
SGPLKAEIAQRLEDVKKKKAVKKYLAAVLGKRYKQRVKNK

SEQ ID NO: 148 - protein, 34 aa, Artificial Sequence
  Synthetic peptide
  PEPTIDE (1)..(9): Influenza NSP1 Th
  PEPTIDE (10)..(13): epsilon-K-KKK spacer
  SITE (10)..(10): epsilon-K
  PEPTIDE (14)..(34): PACAP38 (18-38)
DRLRRDQKSKKKKAVKKYLAAVLGKRYKQRVKNK

SEQ ID NO: 149 - protein, 44 aa, Artificial Sequence
  Synthetic peptide
  PEPTIDE (1)..(19): EBV BHRF1 Th
  PEPTIDE (20)..(23): epsilon-K-KKK spacer
  SITE (20)..(20): epsilon-K
  PEPTIDE (24)..(44): PACAP38 (18-38)
AGLTLSLLVICSYLFISRGKKKKAVKKYLAAVLGKRYKQRVKNK

SEQ ID NO: 150 - protein, 40 aa, Artificial Sequence
  Synthetic peptide
  PEPTIDE (1)..(15): Clostridium tetani TT1 Th
  PEPTIDE (16)..(19): epsilon-K-KKK spacer
  SITE (16)..(16): epsilon-K
  PEPTIDE (20)..(40): PACAP38 (18-38)
QYIKANSKFIGITELKKKKAVKKYLAAVLGKRYKQRVKNK

SEQ ID NO: 151 - protein, 45 aa, Artificial Sequence
  Synthetic peptide
  PEPTIDE (1)..(20): EBV EBNA-1 Th
  PEPTIDE (21)..(24): epsilon-K-KKK spacer
  SITE (21)..(21): epsilon-K
  PEPTIDE (25)..(45): PACAP38 (18-38)
PGPLRESIVCYFMVFLQTHIKKKKAVKKYLAAVLGKRYKQRVKNK

SEQ ID NO: 152 - protein, 46 aa, Artificial Sequence
  Synthetic peptide
  PEPTIDE (1)..(21): Clostridium tetani TT2 Th
  PEPTIDE (22)..(25): epsilon-K-KKK spacer
  SITE (22)..(22): epsilon-K
  PEPTIDE (26)..(46): PACAP38 (18-38)
FNNFTVSFWLRVPKVSASHLEKKKKAVKKYLAAVLGKRYKQRVKNK

SEQ ID NO: 153 - protein, 41 aa, Artificial Sequence
  Synthetic peptide
  PEPTIDE (1)..(16): Clostridium tetani TT3 Th
  PEPTIDE (17)..(20): epsilon-K-KKK spacer
  SITE (17)..(17): epsilon-K
  PEPTIDE (21)..(41): PACAP38 (18-38)
KFIIKRYTPNNEIDSFKKKKAVKKYLAAVLGKRYKQRVKNK

SEQ ID NO: 154 - protein, 41 aa, Artificial Sequence
  Synthetic peptide
  PEPTIDE (1)..(16): Clostridium tetani TT4 Th
  PEPTIDE (17)..(20): epsilon-K-KKK spacer
  SITE (17)..(17): epsilon-K
  PEPTIDE (21)..(41): PACAP38 (18-38)
VSIDKFRIFCKALNPKKKKKAVKKYLAAVLGKRYKQRVKNK

SEQ ID NO: 155 - protein, 43 aa, Artificial Sequence
  Synthetic peptide
  PEPTIDE (1)..(18): EBV CP Th
  PEPTIDE (19)..(22): epsilon-K-KKK spacer
  SITE (19)..(19): epsilon-K
  PEPTIDE (23)..(43): PACAP38 (18-38)
VPGLYSPCRAFFNKEELLKKKKAVKKYLAAVLGKRYKQRVKNK

SEQ ID NO: 156 - protein, 39 aa, Artificial Sequence
  Synthetic peptide
  PEPTIDE (1)..(14): HCMV IE1 Th
  PEPTIDE (15)..(18): epsilon-K-KKK spacer
  SITE (15)..(15): epsilon-K
  PEPTIDE (19)..(39): PACAP38 (18-38)
DKREMWMACIKELHKKKKAVKKYLAAVLGKRYKQRVKNK

SEQ ID NO: 157 - protein, 40 aa, Artificial Sequence
  Synthetic peptide
  PEPTIDE (1)..(15): EBV GP340 Th
  PEPTIDE (16)..(19): epsilon-K-KKK spacer
  SITE (16)..(16): epsilon-K
  PEPTIDE (20)..(40): PACAP38 (18-38)
TGHGARTSTEPTTDYKKKKAVKKYLAAVLGKRYKQRVKNK

SEQ ID NO: 158 - protein, 38 aa, Artificial Sequence
  Synthetic peptide
  PEPTIDE (1)..(13): EBV BPLF1 Th
  PEPTIDE (14)..(17): epsilon-K-KKK spacer
  SITE (14)..(14): epsilon-K
  PEPTIDE (18)..(38): PACAP38 (18-38)
KELKRQYEKKLRQKKKKAVKKYLAAVLGKRYKQRVKNK

SEQ ID NO: 159 - protein, 36 aa, Artificial Sequence
  Synthetic peptide
  PEPTIDE (1)..(11): EBV EBNA-2 Th
  PEPTIDE (12)..(15): epsilon-K-KKK spacer
  SITE (12)..(12): epsilon-K
  PEPTIDE (16)..(36): PACAP38 (18-38)
TVFYNIPPMPLKKKKAVKKYLAAVLGKRYKQRVKNK

SEQ ID NO: 160 - protein, 16 aa, Measles virus
  PEPTIDE (1)..(16): MvF Th
DLSDLKGLLLHKLDGL

SEQ ID NO: 161 - protein, 16 aa, Measles virus
  PEPTIDE (1)..(16): MvF Th
EISDEIRLIIIKRIEI

SEQ ID NO: 162 - protein, 16 aa, Measles virus
  PEPTIDE (1)..(16): MvF Th
DVSDVKGVVVHKVDGV

SEQ ID NO: 163 - protein, 16 aa, Measles virus
  PEPTIDE (1)..(16): MvF Th
DFSDFKGFFFHKFDGF

SEQ ID NO: 164 - protein, 16 aa, Measles virus
  PEPTIDE (1)..(16): MvF Th
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 165 - protein, 19 aa, Measles virus
  PEPTIDE (1)..(19): MvF3 Th
ISISEIKGVIVHKIEGILF

SEQ ID NO: 166 - protein, 19 aa, Measles virus
  PEPTIDE (1)..(19): MvF 3Th
ISITEIRTVIVTRIETILF

SEQ ID NO: 167 - protein, 19 aa, Measles virus
  PEPTIDE (1)..(19): MvF3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIEXILF

SEQ ID NO: 168 - protein, 22 aa, Measles virus
  PEPTIDE (1)..(22): KKKMvF5 Th (UBITh1a)
KKKISITEIKGVIVHRIETILF

SEQ ID NO: 169 - protein, 15 aa, Hepatitis B virus
  PEPTIDE (1)..(15): HBsAg4 Th (UBITh4)
FFLLTRILTIPQSLD

SEQ ID NO: 170 - protein, 18 aa, Hepatitis B virus
  PEPTIDE (1)..(18): KKK-HBsAg Th
KKKFFLLTRILTIPQSLD

SEQ ID NO: 171 - protein, 14 aa, Hepatitis B virus
  PEPTIDE (1)..(14): HBsAg Th
FFLLTRILTIPQSL

SEQ ID NO: 172 - DNA, 32 bp, Artificial Sequence
  CpG1 oligonucleotide ODN
tcgtcgttttgtcgttttgtcgttttgtcgtt

SEQ ID NO: 173 - DNA, 24 bp, Artificial Sequence
  CpG2 oligonucleotide ODN
  misc_feature (1)..(1): phosphorothioate group
tcgtcgttttgtcgttttgtcgtt

SEQ ID NO: 174 - DNA, 24 bp, Artificial Sequence
  CpG3 Oligonucleotide ODN
tcgtcgttttgtcgttttgtcgtt

SEQ ID NO: 175 - protein, 38 aa, Gallus gallus
  PEPTIDE (1)..(38): PACAP chicken
HIDGIFTDSYSRYRKQMAVKKYLAAVLGKRYKQRVKNK

SEQ ID NO: 176 - protein, 33 aa, Heloderma suspectum
  PEPTIDE (1)..(33): PACPA lizard
HSDGIFTDSYSRYRKQMAVKKYLAAVLGKRYKQ

SEQ ID NO: 177 - protein, 38 aa, Pelophylax ridibundus
  PEPTIDE (1)..(38): PACAP green frog
HSDGIFTDSYSRYRKQMAVKKYLAAVLGKRYKQRIKNK

SEQ ID NO: 178 - protein, 38 aa, Oncorhynchus nerka
  PEPTIDE (1)..(38): PACAP salmon
HSDGIFTDSYSRYRKQMAVKKYLAAVLGKRYRQRYRNK

SEQ ID NO: 179 - protein, 38 aa, Clarias macrocephalus
  PEPTIDE (1)..(38): PACAP catfish
HSDGIFTDSYSRYRKQMAVKKYLAAVLGRRYRQRFRNK

SEQ ID NO: 180 - protein, 38 aa, Uranoscopus japonicus
  PEPTIDE (1)..(38): PACAP stargazer
HSDGIFTDSYSRYRKQMAVQKYLAAVLGRRYRQRVRNK

SEQ ID NO: 181 - protein, 44 aa, Dasyatis akajei
  PEPTIDE (1)..(44): PACAP stingray
HSDGIFTDSYSRYRKQMAVKKYLAAVLGKRYKPKVKNSGRRVFY

SEQ ID NO: 182 - protein, 27 aa, Chelysoma productum
  PEPTIDE (1)..(27): PACAP tunicate-I
HSDGIFTDSYSRYRNQMAVKKYLAAVL

SEQ ID NO: 183 - protein, 27 aa, Chelysoma productum
  PEPTIDE (1)..(27): PACAP tunicate-II
HSDGIFTDSYSRYRNQMAVKKYINALL

SEQ ID NO: 184 - protein, 28 aa, Homo sapiens
  PEPTIDE (1)..(28): VIP human/monkey/pig/cattle/sheep/goat/dog/ rabbit/rat/mouse
HSDAVFTDNYTRLRKQMAVKKYLNSILN

SEQ ID NO: 185 - protein, 28 aa, Cavia porcellus
  PEPTIDE (1)..(28): VIP guinea pig
HSDALFTDTYTRLRKQMAMKKYLNSVLN

SEQ ID NO: 186 - protein, 28 aa, Didelphis virginiana
  PEPTIDE (1)..(28): VIP opossum
HSDAVFTDSYTRLLKQMAMRKYLDSILN

SEQ ID NO: 187 - protein, 28 aa, Gallus gallus
  PEPTIDE (1)..(28): VIP chick/turk/allig/frog
HSDAVFTDNYSRFRKQMAVKKYLNSVLT

SEQ ID NO: 188 - protein, 28 aa, Gadus morhua
  PEPTIDE (1)..(28): VIP cod
HSDAVFTDNYSRFRKQMAAKKYLNSVLA

SEQ ID NO: 189 - protein, 28 aa, Oncorhynchus mykiss
  PEPTIDE (1)..(28): VIP trout/bowfin
HSDAIFTDNYSRFRKQMAVKKYLNSVLT

SEQ ID NO: 190 - protein, 28 aa, Scyliorhinus canicula
  PEPTIDE (1)..(28): VIP dogfish
HSDAVFTDNYSRIRKQMAVKKYINSLLA

SEQ ID NO: 191 - protein, 27 aa, Homo sapiens
  PEPTIDE (1)..(27): Secretin human
HSDGTFTSELSRLREGARLQRLLQGLV

SEQ ID NO: 192 - protein, 27 aa, Sus scrofa
  PEPTIDE (1)..(27): Secretin pig/sheep/cattle/guinea pig
HSDGTFTSELSRLRDSARLQRLLQGLV

SEQ ID NO: 193 - protein, 27 aa, Canis lupus
  PEPTIDE (1)..(27): Secretin dog
HSDGTFTSELSRLRESARLQRLLQGLV

SEQ ID NO: 194 - protein, 27 aa, Oryctolagus cuniculus
  PEPTIDE (1)..(27): Secretin rabbit
HSDGTFTSELSRLRDRARLQRLLQGLL

SEQ ID NO: 195 - protein, 27 aa, Rattus norvegicus
  PEPTIDE (1)..(27): Secretin rat
HSDGTFTSELSRLQDSARLQRLLQGLV

SEQ ID NO: 196 - protein, 27 aa, Gallus gallus
  PEPTIDE (1)..(27): Secretin chicken
HSDGLFTSEYSKMRGNAQVQKFIQNLM

SEQ ID NO: 197 - protein, 55 aa, Artificial Sequence
  Synthetic Peptide
  PEPTIDE (11)..(48): Human/Rat/Mouse PACAP38
GDDAEPLSKRHSDGIFTDSYSRYRKQMAVKKYLAAVLGKRYKQRVKNKGRRIAYL